(19) United States
(12) Patent Application Publication
(10) Pub. No.: US 2025/0135444 A1
(43) Pub. Date: May 1, 2025

(54) SYSTEMS AND METHODS FOR MANUFACTURING ELECTROCHROMICS

(71) Applicant: Kennesaw State University Research And Service Foundation, Inc., Kennesaw, GA (US)

(72) Inventors: Graham Collier, Kennesaw, GA (US); Allison Hawks, Acworth, GA (US)

(21) Appl. No.: 18/932,167

(22) Filed: Oct. 30, 2024

Related U.S. Application Data
(60) Provisional application No. 63/594,386, filed on Oct. 30, 2023.

(51) Int. Cl. B01J 31/02; B01J 23/72; C07C 37/02

(52) U.S. Cl. CPC B01J 31/0244; B01J 23/72; C07C 37/02

(57) ABSTRACT

The present disclosure relates to a variety of DHPP-based electrochromes. Such electrochromes include a DHPP scaffold with a variety of end groups coupled to the periphery of the scaffold. Such end groups can be substituted for other end groups if desired. End groups include groups of various electron-donating characters. DHPP-based electrochromes are useful as anodically coloring electrochromes capable of being incorporated into a variety of optoelectronic applications.

### CROSS-REFERENCE TO RELATED APPLICATIONS

This application is a Nonprovisional of and claims the benefit of U.S. Provisional Application No. 63/594,386, filed Oct. 30, 2023, which is hereby incorporated by reference in its entirety for any and all purposes.

### BACKGROUND

Prior efforts to create π-expanded DHPPs have involved intramolecular couplings that yielded fused aromatic chromophores. Tasior, M., et al., J. Org. Chem. 85: 13529-13543 (2020). Tasior et al. reported a cross-metathesis/borylation procedure to create a π-extended DHPP followed by a Sonogashira coupling reaction to tune the fluorescence of the molecule. Tasior, M., et al., Chem. Sci., 12(48): 15935-15946 (2021). Tasior et al. used a Sonogashira reaction to produce DHPPs functionalized through the 3,6-positions of the pyrrolopyrrole scaffold. Tasior, M., et al., Chem. Eur. J., 25(2): 598-608 (2019). Sonogashira and Suzuki cross couplings also have been used to synthesize π-extended DHPPs that displayed two-photon absorbances or that were used in dye-sensitized solar cells (DSSCs). Wang, J., et al., Chem. Eur. J., 24(68): 18032-18042 (2018); Janiga, A., et al., Org. Biomol. Chem., 12(18): 2874-2881 (2014).

Krzeszewski et al. and Ryu et al. used direct arylation to obtain DHPP analogs functionalized through the 3,6-positions. The chromophores reported in these works displayed high fluorescence quantum yields that potentially useful as fluorescent dyes for suited applications. Krzeszewski, M., et al., J. Org. Chem., 79: 3119-3128 (2014); Ryu, H. G., et al., J. Phys. Chem. C, 122(25): 13424-13434 (2018). Still, the scope of coupling partners remains limited, and most reaction yields were modest for intramolecular, Krzeszewski, M., et al., Chem. Eur. J., 22(46): 16478-16488 (2016); Tasior, M., et al., Chem. Sci., 12(48): 15935-15946 (2021); Krzeszewski, M., et al., Acc. Chem. Res., 50(9): 2334-2345 (2017). Sonogashira, Suzuki, and direct arylation coupling reactions. Janiga, A., et al., Org. Biomol. Chem., 12(18): 2874-2881 (2014); Tasior, M., et al., Chem. Eur. J., 25(2): 598-608 (2019); Liu, H., et al., Tetrahedron Lett., 58(52): 4841-4844 (2017); Ryu, H. G., et al., J. Phys. Chem. C, 122(25): 13424-13434 (2018).

While these efforts highlight the ability to functionalize DHPPs through metal-catalyzed reactions, there still is a need to expand the structural diversity of building blocks that efficiently couple with DHPP in these types of reactions or even in high-yielding polymerizations. A thorough understanding of how functionalities influence application-inspired properties is concurrently needed.

Additionally, current high-contrast anodically coloring electrochromes are limited to discrete molecules that are not easily amenable to solution processing and the chemical design space is sparse. With these drawbacks in mind, it is necessary to discover new materials.

### SUMMARY

The present disclosure relates to chromophores and studies thereof.

The present disclosure additionally relates to electrochromes. Electrochromes include anodically coloring electrochromes. Colors of electrochromes described herein are capable of being selected, tuned, controlled, and then like. Such selection, tuning, controlling, and the like may be accomplished by altering peripheral or end groups attached to a molecular scaffold. The present disclosure relates to color-controlled, high-contrast electrochromes. Color of electrochromes may additionally be controlled by subjecting such electrochromes to oxidizing or reducing environments.

The present disclosure relates to electrochromes including one or more molecular scaffolds. Such scaffolds are capable of being coupled to one or more periphery or end groups. A non-limiting example of a molecular scaffold includes DHPP or DHPP derivatives. Periphery or end groups of various electron-donating characteristics may be attached to a molecular scaffold. Periphery or end groups include but are not limited to monocyclic, bicyclic aryl, heteroaryl group, and substituted varieties thereof. Substitutions may include substituting aliphatic, alkenyl, alkynyl, ether, halogenated alkane, cyano, or halogen groups onto periphery or end groups.

The present disclosure relates to methods of synthesis of chromophores and electrochromes. Such synthesis may include Suzuki cross-coupling reactions. Synthesis of chromophores and electrochromes may be considered synthetically simple.

The present disclosure relates to applications of chromophores and electrochromes. Applications include but are not limited to incorporating chromophores and electrochromes into polymers, films, and other optoelectronic applications.

### BRIEF DESCRIPTION OF THE DRAWINGS

The following drawings show generally, by way of example, but not by way of limitation, various examples discussed in the present disclosure. In the drawings:

FIG. 1 shows absorbance spectra according to the present disclosure.

FIG. 2 shows a reaction scheme according to the present disclosure.

FIG. 3 shows absorbance spectra according to the present disclosure.

FIG. 4 shows redox responses according to the present disclosure.

FIG. 5 shows solution oxidation spectra according to the present disclosure.

FIG. 6 shows color coordinates according to the present disclosure.

FIG. 7 shows electrochemical switching according to the present disclosure.

FIG. 8 shows tuning according to the present disclosure.

FIG. 9 shows a reaction scheme according to the present disclosure.

FIG. 10 shows a reaction scheme according to the present disclosure.

FIG. 11 shows absorbance spectra according to the present disclosure.

FIG. 12 shows redox responses according to the present disclosure.

FIG. 13 shows solution oxidation spectra according to the present disclosure.

FIG. 14 shows color coordinate data according to the present disclosure.

FIG. 15 shows electrochemical switching according to the present disclosure.

FIG. 16 shows DHPP-containing polymers according to the present disclosure.

FIG. 17 shows DHPPs and anodically coloring electrochromes compared with synthesis according to the present disclosure.

FIG. 18 shows absorbance spectra according to the present disclosure.

FIG. 19 shows a reaction scheme according to the present disclosure.

FIG. 20 shows cyclic voltammetry according to the present disclosure.

FIG. 21 shows solution oxidation studies according to the present disclosure.

FIG. 22 shows color coordinates according to the present disclosure.

FIG. 23 shows electrochemical switching according to the present disclosure.

FIG. 24 shows a reaction scheme according to the present disclosure.

FIG. 25 shows reaction schemes according to the present disclosure.

FIG. 26 shows absorbance spectra according to the present disclosure.

FIG. 27 shows reaction schemes according to the present disclosure.

FIG. 28 shows absorbance spectra according to the present disclosure.

FIG. 29 shows redox studies using cyclic voltammetry according to the present disclosure.

FIG. 30 shows redox studies according to the present disclosure.

FIG. 31 shows solution oxidation studies according to the present disclosure.

FIG. 32 shows colorimetry data according to the present disclosure.

FIG. 33 shows absorbance spectra according to the present disclosure.

FIG. 34 shows redox studies according to the present disclosure.

FIG. 35 shows an energy level diagram according to the present disclosure.

FIG. 36 shows absorbance spectra according to the present disclosure.

FIG. 37 shows solution oxidation studies according to the present disclosure.

FIG. 38 shows CV from −0.4 V to 2 V of DHPP molecules included within this disclosure.

FIG. 39 shows DPV traces of the five color-controlled DHPPs according to this disclosure with their onsets of oxidation including A) 4-FDHPP, B) F,OMeDHPP, C) 4-OMeDHPP, D) 4-SMeDHPP, and E) 4-tol2ADHPP using an Ag/AgCl reference electrode and a 0.5 M TBAPF6/DCM supporting electrolyte.

FIG. 40 shows colorimetry data for all ACE DHPP electrochromes according to this disclosure. Each neutral molecule included here displayed a colorless neutral solution and upon oxidation transitioned into the green-yellow region of the color quadrants.

### DETAILED DESCRIPTION

The present disclosure can be understood more readily by reference to the following detailed description, examples, drawings, and claims, and their previous and following description. However, before the present compositions, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

### I. Definitions

It should be appreciated that this disclosure is not limited to the compositions and methods described herein. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

Unless defined otherwise, all composition percentage values used herein are given in terms of weight percentage.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or example language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the term “alkyl” refers to an unsubstituted or substituted linear or branched alkyl group containing the indicated number of carbon atoms. If no number is indicated, then alkyl (optionally including any substituents on alkyl) may contain 1 to 16 carbon atoms. Preferably, the alkyl group contains 1 to 10 carbon atoms, alternatively 1 to 7 carbon atoms, or alternatively 1 to 4 carbon atoms. Examples of alkyl include methyl, ethyl, propyl, isopropyl, butyl, iso-, sec- and tert-butyl, pentyl, hexyl, heptyl, 3-ethylbutyl, and the like. Examples of substituents on alkyl include 1, 2, or 3 groups independently selected from hydroxy, amino, amido, oxa, carboxy, alkyl carboxy, carbonyl, alkoxy, amido, carbamate, carbonate, halogen, phenyl, benzyl, thiol, and combinations thereof. “Alkylene” means a divalent alkyl group, such as —CH2-, —CH2CH2-, —CH2CH2CH2-, —CH2CH(CH3)CH2-, and —CH2CH2CH2CH2-.

“Haloalkyl” refers to an alkyl group as defined above substituted with one or more halogen atoms, where each halogen is independently F, Cl, Br or I. A preferred halogen is F. Preferred haloalkyl groups contain 1-6 carbons, more preferably 1-4 carbons, and still more preferably 1-2 carbons. “Haloalkyl” includes perhaloalkyl groups, such as —CF3- or —CF2CF3-. “Haloalkylene” means a divalent haloalkyl group, such as —CH2CF2-.

The terms “halogen” or “halo” indicate fluorine, chlorine, bromine, and iodine.

“Cycloalkyl” refers to an unsubstituted or substituted cyclic hydrocarbon containing the indicated number of ring carbon atoms. If no number is indicated, then cycloalkyl may contain 3 to 12 ring carbon atoms. Preferred are C3-C8 cycloalkyl groups, C3-C7 cycloalkyl, more preferably C4-C7 cycloalkyl, and still more preferably C5-C6 cycloalkyl. Examples of cycloalkyl include cyclopropyl, cyclobutyl, cyclopentyl, cyclohexyl, cycloheptyl and cyclooctyl. Examples of substituents on cycloalkyl include 1, 2, or 3 groups independently selected from alkyl, hydroxy, amino, amido, oxa, carbonyl, alkoxy, amido, carbamate, carbonate, halo, phenyl, benzyl, and combinations thereof. “Cycloalkylene” means a divalent cycloalkyl group, such as 1,2-cyclohexylene, 1,3-cyclohexylene, or 1,4-cyclohexylene.

“Heterocycloalkyl” refers to a cycloalkyl ring or ring system as defined above in which at least one ring carbon has been replaced with a heteroatom selected from nitrogen, oxygen, and sulfur. The heterocycloalkyl ring is optionally fused to or otherwise attached to other heterocycloalkyl rings and/or non-aromatic hydrocarbon rings and/or phenyl rings. Preferred heterocycloalkyl groups have from 5 to 7 members. More preferred heterocycloalkyl groups have 5 or 6 members. Heterocycloalkylene means a divalent heterocycloalkyl group.

“Aryl” refers to an unsubstituted or substituted aromatic hydrocarbon ring system containing at least one aromatic ring. The aryl group contains the indicated number of ring carbon atoms. If no number is indicated, then aryl may contain 6 to 14 ring carbon atoms. The aromatic ring may optionally be fused or otherwise attached to other aromatic hydrocarbon rings or non-aromatic hydrocarbon rings. Examples of aryl groups include phenyl, naphthyl, and biphenyl. Preferred examples of aryl groups include phenyl. Examples of substituents on aryl include 1, 2, or 3 groups independently selected from alkyl, hydroxy, amino, amido, oxa, carboxy, alkyl carboxy, carbonyl, alkoxy, amido, carbamate, carbonate, halo, phenyl, benzyl, and combinations thereof. “Arylene” means a divalent aryl group, for example 1,2-phenylene, 1,3-phenylene, or 1,4-phenylene.

“Heteroaryl” refers to an aryl ring or ring system, as defined above, in which at least one ring carbon atom has been replaced with a heteroatom selected from nitrogen, oxygen, and sulfur. The heteroaryl ring may be fused or otherwise attached to one or more heteroaryl rings, aromatic or nonaromatic hydrocarbon rings or heterocycloalkyl rings. Examples of heteroaryl groups include pyridyl, furyl, and thienyl. “Heteroarylene” means a divalent heteroaryl group.

“Alkoxy” refers to an alkyl group attached to the parent molecular moiety through an oxygen bridge. Examples of alkoxy groups include, for instance, methoxy, ethoxy, propoxy and isopropoxy. “Aryloxy” refers to an aryl group attached to a parent molecular moiety through an oxygen bridge. Examples include phenoxy. “Cyclic alkoxy” means a cycloalkyl group attached to the parent moiety through an oxygen bridge.

“Alkylamine” refers to an alkyl group attached to the parent molecular moiety through an —NH bridge. Alkyleneamine means a divalent alkylamine group, such as —CH2CH2NH—.

“Ester” refers to a class of organic compounds having the general formula RCOOR′, wherein R and R′ are any organic combining groups. R and R′ may be selected from functional groups comprising alkyls, substituted alkyls, alkylene, haloalkyls, cycloalkyls, heterocyloalkyls, aryls, heteroaryls, alkoxys, cycloalkoxys, alkylamines, siloxanyls, silyls, alkyleneoxys, oxaalkylenes, and the like. Definitions for the above mentioned functional groups are provided herein.

As used herein, “esterification” refers to a reaction producing an ester. The reaction often involves an alcohol and a Bronsted acid (such as a carboxylic acid, sulfuric acid, or phosphoric acid). Furthermore, the term “transesterification” refers to the reaction of an alcohol molecule and a pre-existing ester molecule react to form a new ester. In some aspects, transesterification can be mediated by other compounds, such as carbonyldiimidazole.

“Siloxanyl” refers to a structure having at least one Si—O—Si bond. Thus, for example, siloxanyl group means a group having at least one Si—O—Si group (i.e. a siloxane group), and siloxanyl compound means a compound having at least one Si—O—Si group. “Siloxanyl” encompasses monomeric (e.g., Si—O—Si) as well as oligomeric/polymeric structures (e.g., —[Si—O]n-, where n is 2 or more). Each silicon atom in the siloxanyl group is substituted with independently selected RA groups (where RA is as defined in formula A options (b)-(i)) to complete their valence.

“Silyl” refers to a structure of formula R3Si and “siloxy” refers to a structure of formula R3Si—O—, where each R in silyl or siloxy is independently selected from trimethylsiloxy, C1-C8 alkyl (preferably C1-C3 alkyl, more preferably ethyl or methyl), and C3-C8 cycloalkyl.

“Alkyleneoxy” refers to groups of the general formula -(alkylene-O)p- or —(O-alkylene)p-, wherein alkylene is as defined above, and p is from 1 to 200, or from 1 to 100, or from 1 to 50, or from 1 to 25, or from 1 to 20, or from 1 to 10, wherein each alkylene is independently optionally substituted with one or more groups independently selected from hydroxyl, halo (e.g., fluoro), amino, amido, ether, carbonyl, carboxyl, and combinations thereof. If p is greater than 1, then each alkylene may be the same or different and the alkyleneoxy may be in block or random configuration. When alkyleneoxy forms a terminal group in a molecule, the terminal end of the alkyleneoxy may, for instance, be a hydroxy or alkoxy (e.g., HO—[CH2CH2O]p- or CH3O—[CH2CH2O]p-). Examples of alkyleneoxy include polymethyleneoxy, polyethyleneoxy, polypropyleneoxy, polybutyleneoxy, and poly(ethyleneoxy-co-propyleneoxy).

“Oxaalkylene” refers to an alkylene group as defined above where one or more non-adjacent CH2 groups have been substituted with an oxygen atom, such as —CH2CH2OCH(CH3)CH2-. “Thiaalkylene” refers to an alkylene group as defined above where one or more non-adjacent CH2 groups have been substituted with a sulfur atom, such as —CH2CH2SCH(CH3)CH2-.

The term “linking group” refers to a moiety that links the polymerizable group to the parent molecule. The linking group may be any moiety that does not undesirably interfere with the polymerization of the compound of which it is a part. For instance, the linking group may be a bond, or it may comprise one or more alkylene, haloalkylene, amide, amine, alkyleneamine, carbamate, carboxylate (—CO2-), disulfide, arylene, heteroarylene, cycloalkylene, heterocycloalkylene, alkyleneoxy, oxaalkylene, thiaalkylene, haloalkyleneoxy (alkyleneoxy substituted with one or more halo groups, e.g., —OCF2-, —OCF2CF2-, —OCF2CH2-), siloxanyl, alkylenesiloxanyl, thiol, or combinations thereof. The linking group may optionally be substituted with 1 or more substituent groups. Suitable substituent groups may include those independently selected from alkyl, halo (e.g., fluoro), hydroxyl, HO-alkyleneoxy, CH3O-alkyleneoxy, siloxanyl, siloxy, siloxy-alkyleneoxy-, siloxy-alkylene-alkyleneoxy- (where more than one alkyleneoxy groups may be present and wherein each methylene in alkylene and alkyleneoxy is independently optionally substituted with hydroxyl), ether, amine, carbonyl, carbamate, and combinations thereof. The linking group may also be substituted with a polymerizable group, such as (meth)acrylate (in addition to the polymerizable group to which the linking group is linked).

Preferred linking groups include C1-C8 alkylene (preferably C2-C6 alkylene) and C1-C8 oxaalkylene (preferably C2-C6 oxaalkylene), each of which is optionally substituted with 1 or 2 groups independently selected from hydroxyl and siloxy. Preferred linking groups also include carboxylate, amide, C1-C8 alkylene-carboxylate-C1-C8 alkylene, or C1-C8 alkylene-amide-C1-C8 alkylene.

As used herein, “pyrrole” refers to a heterocyclic compound (e.g. C4H5N) that has a ring comprising four carbon atoms and one nitrogen atom, polymerizes readily in air, and is the parent compound of many biologically important substances (such as bile pigments, porphyrins, and chlorophyll). The term pyrrole used herein refers to pyrrole (C5H5N), derivatives of pyrrole (e.g., indole), substituted pyrroles, as well as metal pyrrolide compounds.

When the linking group is comprised of combinations of moieties as described above (e.g., alkylene and cycloalkylene), the moieties may be present in any order. For instance, if in Formula E below, L is indicated as being -alkylene-cycloalkylene-, then Rg-L may be either Rg-alkylene-cycloalkylene-, or Rg-cycloalkylene-alkylene-. Notwithstanding this, the listing order represents the preferred order in which the moieties appear in the compound starting from the terminal polymerizable group (Rg) to which the linking group is attached. For example, if in Formula E, L and L2 are indicated as both being alkylene-cycloalkylene, then Rg-L is preferably Rg-alkylene-cycloalkylene- and -L2-Rg is preferably -cycloalkylene-alkylene-Rg.

As used herein, a “coupling reaction” is a reaction or reaction sequence in which the net reaction is the connection of carbon skeletons of two compounds containing a common functional group.

As used herein, “oxidation” refers to a chemical process by which an atom of an element gains bonds to more electronegative elements, most commonly oxygen. In this process, the oxidized element increases its oxidation state, which represents the charge of an atom. Oxidation reactions are commonly coupled with “reduction” reactions, wherein the oxidation state of the reduced atom decreases.

As used herein, a “redox reaction” or “reduction oxidation reaction” refers to a type of chemical reaction that involves a transfer of electrons between two species. An oxidation-reduction reaction is any chemical reaction in which the oxidation number of a molecule, atom, or ion changes by gaining or losing an electron.

As used herein, the term “in air” refers to a set of reaction conditions. Such conditions may comprise ambient atmosphere conditions not needing inert gasses to run a reaction. This is advantageous because it eliminates the need for specialty glassware or purchasing nitrogen or argon gas cylinders.

As used herein, the “visible spectrum” refers to a range of wavelengths within the electromagnetic spectrum, wherein the range spans about 380 nm to 700 nm. The visible spectrum may be broken up into different wavelength regions corresponding to colors including red, orange, yellow, green, blue, indigo, and violet. Certain ranges of wavelengths falling in the visible spectrum have been known to cause damage to human eyes.

As used herein, the “ultraviolet (UV) spectrum” refers to a range of wavelengths within the electromagnetic spectrum, wherein the range spans about 10 nm to 400 nm.

As used herein, “light absorption” is defined as the phenomenon wherein electrons absorb the energy of incoming light waves (i.e. photons) and change their energy state. In order for this to occur, the incoming light waves must be at or near the energy levels of the electrons. The resultant absorption patterns characteristic to a given material may be displayed using an “absorption spectrum”, wherein an “absorption spectrum” shows the change in absorbance of a sample as a function of the wavelength of incident light and may be measured using a spectrophotometer. Unique to an “absorption spectrum” is an “absorption peak”, wherein the frequency or wavelength of a given sample exhibits the maximum or the highest spectral value of light absorption. With regards to light absorption of wavelengths of light corresponding to the visible spectrum, a material or matter absorbing light waves of certain wavelengths of the visible spectrum may cause an observer to not see these wavelengths in the reflected light.

As used herein, “fluorescence” refers to a type of luminescence that occurs in gas, liquid or solid matter. Fluorescence occurs following the absorption of light waves (i.e. photons), which may promote an electron from the ground state promoted to an excited state. In fluorescence, the spin of the electron is still paired with the ground state electron, unlike phosphorescence. As the excited electron returns to the ground state, it emits a photon of lower energy, corresponding to a longer wavelength, than that of the absorbed photon.

As used herein, “phosphorescence” refers to a phenomenon of delayed luminescence that corresponds to the radiative decay of an excited electron from the molecular triplet state. As a general property, phosphorescence represents a challenge of chemical physics due to the spin prohibition of the underlying triplet-singlet photon emission and because its analysis embraces a deep knowledge of electronic molecular structure. Phosphorescence is the simplest physical process which provides an example of spin-forbidden transformation with a characteristic spin selectivity and magnetic field dependence, being the model also for more complicated chemical reactions and for spin catalysis applications. Phosphorescence is commonly viewed as the alternative method of photon emission with regards to fluorescence. Methods exist in the art to increase the amount of fluorescence versus phosphorescence emission, such as the use of heavy metals to increase spin coupling.

As used herein, the “quantum yield (Φ)” is a measure of the efficiency of photon emission as defined by the ratio of the number of photons emitted to the number of photons absorbed.

As used herein, “anisotropic” describes a material wherein a given property of said material depends on the direction in which it is measured. Moreover, something that is “anisotropic” changes in size or in its physical properties according to the direction in which it is measured. Examples of anisotropic materials may comprise graphite, carbon fiber, nanoparticles, etc.

As used herein, “isotropic” describes a material wherein a given property of said material does not depend on the direction in which it is measured. Moreover, something that is “isotropic” remains constant in size or in its physical properties according to the direction in which it is measured.

As used herein, “ligand” refers to an ion or neutral molecule that bonds to a central metal atom or ion. Example ligands may comprise PVP, PVA, DSDMA, and other molecules capable of bonding to a central metal atom. Metal atoms may comprise a variety of metals including, but not limited to, noble metals such as gold. Ligands have at least one donor with an electron pair used to form covalent bonds with the metal central atom.

As used herein, an “intermediate ligand” refers to a ligand temporarily conjugated to a metal atom that is further exchanged to allow the conjugation of an alternate ligand.

As used herein, the terms “physical absorption” or “chemical absorption” refer to processes in which atoms, molecules, or particles enter the bulk phase of a gas, liquid, or solid material and are taken up within the volume. Absorption in this manner may be driven by solubility, concentration gradients, temperature, pressure, and other driving forces known in the art.

As used herein, “adsorption” is defined as the deposition of a species onto a surface. The species that gets adsorbed on a surface is known as an adsorbate, and the surface on which adsorption occurs is known as an adsorbent. Examples of adsorbents may comprise clay, silica gel, colloids, metals, nanoparticles etc. Adsorption may occur via chemical or physical adsorption. Chemical adsorption may occur when an adsorbate is held to an adsorbent via chemical bonds, whereas physical adsorption may occur when an adsorbate is joined to an adsorbent via weak van der Waal's forces.

As used herein, “colloid” refers to dispersions of wherein one substance is suspended in another. Many examples of colloids in the art contain polymers. In this aspect, polymers may be adsorbed or chemically attached to the surface of particles suspended in the colloid, or the polymers may freely move in the colloidal suspension. The presence of polymers on particles in the suspension may directly relate to “colloidal stability,” wherein “colloidal stability” refers to the tendency of a colloidal suspension to undergo sedimentation. Sedementation would result in the falling of particles out of a colloid. Polymers adsorbed or chemically attached to a particle may affect its colloidal stability.

As used herein, the term “diffusion” refers to the process wherein there is a net flow of matter from one region to another. An example of such process is “surface diffusion,” wherein particles move from one area of the surface of a subject to another area of the same surface. This can be caused by thermal stress or applied pressure.

As used herein, a “surfactant” refers to a substance that, when added to a liquid, reduces its surface tension, thereby increasing its spreading and wetting properties. Typical surfactants may be partly hydrophilic and partly lipophilic.

As used herein, the term “wetting agent” refers to a specific class of surfactant, wherein a wetting agent reduces the surface tension of water and thus allows a liquid to more easy spread on or “wet” a surface. The high surface tension of water causes problems in many industrial processes where water-based solutions are used, as the solution is not able to wet the surface it is applied to. Wetting agents are commonly used to reduce the surface tension of water and thus help the water-based solutions to spread.

“Target macromolecule” means the macromolecule being synthesized from the reactive monomer mixture comprising monomers, macromers, prepolymers, cross-linkers, initiators, additives, diluents, and the like.

The term “polymerizable compound” means a compound containing one or more polymerizable groups. The term encompasses, for instance, monomers, macromers, oligomers, prepolymers, cross-linkers, and the like.

“Polymerizable groups” are groups that can undergo chain growth polymerization, such as free radical and/or cationic polymerization, for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyl lactam, N-vinylamide, and styryl functional groups, and mixtures of any of the foregoing. More preferably, the free radical polymerizable groups comprise (meth)acrylates, (meth)acrylamides, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

A “monomer” is a mono-functional molecule which can undergo chain growth polymerization, and in particular, free radical polymerization, thereby creating a repeating unit in the chemical structure of the target macromolecule. Some monomers have di-functional impurities that can act as cross-linking agents. A “hydrophilic monomer” is also a monomer which yields a clear single phase solution when mixed with deionized water at 25° C. at a concentration of 5 weight percent. A “hydrophilic component” is a monomer, macromer, prepolymer, initiator, cross-linker, additive, or polymer which yields a clear single phase solution when mixed with deionized water at 25° C. at a concentration of 5 weight percent. A “hydrophobic component” is a monomer, macromer, prepolymer, initiator, cross-linker, additive, or polymer which is slightly soluble or insoluble in deionized water at 25° C.

A “macromolecule” is an organic compound having a number average molecular weight of greater than 1500, and may be reactive or non-reactive.

A “macromonomer” or “macromer” is a macromolecule that has one group that can undergo chain growth polymerization, and in particular, free radical polymerization, thereby creating a repeating unit in the chemical structure of the target macromolecule. Typically, the chemical structure of the macromer is different than the chemical structure of the target macromolecule, that is, the repeating unit of the macromer's pendent group is different than the repeating unit of the target macromolecule or its mainchain. The difference between a monomer and a macromer is merely one of chemical structure, molecular weight, and molecular weight distribution of the pendent group. As a result, and as used herein, the patent literature occasionally defines monomers as polymerizable compounds having relatively low molecular weights of about 1,500 Daltons or less, which inherently includes some macromers. In particular, monomethacryloxypropyl terminated mono-n-butyl terminated polydimethylsiloxane (molecular weight=500-1500 g/mol) (mPDMS) and mono-(2-hydroxy-3-methacryloxypropyl)-propyl ether terminated mono-n-butyl terminated polydimethylsiloxane (molecular weight=500-1500 g/mol) (OH-mPDMS) may be referred to as monomers or macromers. Furthermore, the patent literature occasionally defines macromers as having one or more polymerizable groups, essentially broadening the common definition of macromer to include prepolymers. As a result, and as used herein, di-functional and multi-functional macromers, prepolymers, and crosslinkers may be used interchangeably.

A “polymer” is a target macromolecule composed of the repeating units of the monomers used during polymerization. Example polymers may comprise poly(ethylene glycol) (PEG), polycarbonate, poly(vinyl alcohol) (PVA), polyvinylpyrrolidone (PVP), polystyrene (PS), polycaprolactone (PCL), ethylene oligomers or polyethylene (PE), polypropylene (PP), poly(methyl methacrylate) (PMMA), and other polymers known in the art.

As used herein, the term “thermoplastic” refers to a property of polymers, wherein the polymer may be melted, solidified, and then successfully melted and solidified again. This process may be repeated several times for thermoplastic polymers without loss of functionality.

As used herein, the term “thermoset” refers to a property of polymers, wherein a thermoset polymer forms well-defined, irreversible, chemical networks that tend to grow in three dimensional directions through the process of curing, which can either occur due to heating or through the addition of a curing agent, therefore causing a crosslinking formation between its chemical components, and giving the thermoset a strong and rigid structure that can be added to other materials to increase strength. Once a thermoset polymer has formed networks during curing, the polymer cannot be re-cured to set in a different manner.

A “homopolymer” is a polymer made from one monomer; a “copolymer” is a polymer made from two or more monomers; a “terpolymer” is a polymer made from three monomers. A “block copolymer” is composed of compositionally different blocks or segments. Diblock copolymers have two blocks. Triblock copolymers have three blocks. “Comb or graft copolymers” are made from at least one macromer.

A “repeating unit” is the smallest group of atoms in a polymer that corresponds to the polymerization of a specific monomer or macromer.

An “initiator” is a molecule that can decompose into radicals which can subsequently react with a monomer to initiate a free radical polymerization reaction. A thermal initiator decomposes at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A photo-initiator decomposes by a photochemical process; typical examples are derivatives of benzil, benzoin, acetophenone, benzophenone, camphorquinone, and mixtures thereof as well as various monoacyl and bisacyl phosphine oxides and combinations thereof.

A “cross-linking agent” is a di-functional or multi-functional monomer or macromer which can undergo free radical polymerization at two or more locations on the molecule, thereby creating branch points and a polymeric network. Common examples are bis(2-methacryloyl)oxyethyl disulfide (DSDMA), cetyltrimethylammonium bromide (CTAB), cetyltrimethylammonium chloride (CTAC), ethylene glycol dimethacrylate, tetraethylene glycol dimethacrylate, trimethylolpropane trimethacrylate, methylene bisacrylamide, triallyl cyanurate, and the like.

A “prepolymer” is a reaction product of monomers which contains remaining polymerizable groups capable of undergoing further reaction to form a polymer.

A “polymeric network” is a cross-linked macromolecule that can swell but cannot dissolve in solvents. “Hydrogels” are polymeric networks that swell in water or aqueous solutions, typically absorbing at least 10 weight percent water. “Silicone hydrogels” are hydrogels that are made from at least one silicone-containing component with at least one hydrophilic component. Hydrophilic components may also include non-reactive polymers.

An “interpenetrating polymeric network” comprises two or more networks which are at least partially interlaced on the molecular scale but not covalently bonded to each other and which cannot be separated without braking chemical bonds. A “semi-interpenetrating polymeric network” comprises one or more networks and one or more polymers characterized by some mixing on the molecular level between at least one network and at least one polymer. A mixture of different polymers is a “polymer blend.” A semi-interpenetrating network is technically a polymer blend, but in some cases, the polymers are so entangled that they cannot be readily removed.

The terms “reactive mixture” and “reactive monomer mixture” refer to the mixture of components (both reactive and non-reactive) which are mixed together and when subjected to polymerization conditions form the conventional or silicone hydrogels of the present invention as well as contact lenses made therefrom. The reactive monomer mixture may comprise reactive components such as the monomers, macromers, prepolymers, cross-linkers, and initiators, additives such as wetting agents, release agents, polymers, dyes, light absorbing compounds such as UV absorbers, pigments, dyes and photochromic compounds, any of which may be reactive or non-reactive but are capable of being retained within the resulting biomedical device, as well as pharmaceutical and nutraceutical compounds, and any diluents. It will be appreciated that a wide range of additives may be added based upon the biomedical device which is made and its intended use. Concentrations of components of the reactive mixture are expressed as weight percentages of all components in the reactive mixture, excluding diluent. When diluents are used, their concentrations are expressed as weight percentages based upon the amount of all components in the reactive mixture and the diluent.

“Reactive components” are the components in the reactive mixture which become part of the chemical structure of the polymeric network of the resulting hydrogel by covalent bonding, hydrogen bonding, electrostatic interactions, the formation of interpenetrating polymeric networks, or any other means.

The term “multi-functional” refers to a component having two or more polymerizable groups. The term “mono-functional” refers to a component having one polymerizable group.

As used herein, “electrochromism” refers to a phenomenon exhibited by certain electroactive materials with reversible and significant visible change in absorbance spectra. Electrochromism further refers to a reversible change in optical properties of a material caused by redox reactions. Redox reactions can be initiated when a material is placed on the surface of an electrode. When an electrochromic material is capable of showing several colors, it is known as “polyelectrochromic”. Changes in color may occur when a chromophore is forced to change its absorption spectrum by the application of electric potential. As a non-limiting example, the absorption may change from the UV region into the visible region.

As used herein, “high-contrast” refers a material that is quantifiably transmissive in its neutral, non-oxidized state (i.e. L*a*b*=100, 0, 0) and transitions to a colored state (e.g., yellow) in its oxidized state. If the transition between the neutral and oxidized states transitions from one color to another color that is significantly different, the molecule is referred to as a “high-contrast” electrochrome.

As used herein, “organic photovoltaics (OPV)” refers to a field focusing on creating devices that convert solar energy to electrical energy. An OPV device may include one or several photoactive materials sandwiched between electrodes.

As used herein, “organic light emitting diodes (OLEDs)” refer to solid-state light devices that use flat light emitting technology in addition to two conductors between which a series of organic thin films are kept.

As used herein, “pi electrons” or “n electrons” refer to electrons residing within certain molecular orbitals. As non-limiting examples, pi electrons may reside in the pi bonds of a double bond, a triple bond, and a conjugated p orbital. As a non-limiting example, the allyl carbanion has four pi electrons.

As used herein, “antibonding pi electrons” or “π* electrons” refer to electrons residing within certain molecular orbitals. As a non-limiting example, antibonding pi electrons reside in antibonding molecular orbitals of pi bonds where antibonding orbitals weaken chemical bonds and raise energies associated therewith. As a non-limiting example, ethylene comprises a π* molecular orbital with four orbital lobes.

As used herein, “frontier molecular orbital theory (FMOT)” refers to the idea of frontier molecular orbitals of a compound at the frontier of electron occupation. This relates to a highest-occupied molecular orbital (HOMO) and a lowest-unoccupied molecular orbital (LUMO).

As used herein, “torsional strain” refers to destabilization of a molecule caused by the eclipsing of groups present on the adjacent atoms. It is the difference in the energy between a staggered and eclipsed conformation and can be qualified using a Newman projection.

As used herein, “transmissive” refers to molecules that do not have color. For a material to be considered “transmissive” or “colorless”, the L* value according to the Commission Internationale de l'Eclairage on L*a*b* (CIELAB) color standard should be equal to 100 while the a* and b* values should be within ±10. Color coordinates described herein are measured according to the CIELAB color standard. For a material to be considered “non-transmissive” or having “color”, it must not be transmissive. That is, such a material should have an L* value not equal to 100 while the a* and b* values should not be within ±10.

Unless otherwise indicated, ratios, percentages, parts, and the like are by weight.

Unless otherwise indicated, numeric ranges, for instance as in “from 2 to 10,” are inclusive of the numbers defining the range (e.g., 2 and 10).

### II. Chromophores and Electrochromes

The present disclosure relates to anodically coloring electrochromes. Anodically coloring electrochromes are preferred over cathodically coloring electrochromes. Anodically coloring electrochromes provide high contrast coloration during electrochemical switching.

The present disclosure relates to anodically coloring electrochromes that are easily solution processed. Ease of solution process provides synthetic accessibility for synthesis of polymers used in electrochromic application. Such electrochromes provide additional solutions over the prior art.

The present disclosure relates to a variety of uses of DHPP-based monomers, monomeric building blocks, copolymers, and films. DHPPs possess desirable characteristics for electrochromic applications. As an example, DHPPs function as high-contrast chromophores with transmissive-to-color or color-to-transmissive properties. DHPPs are π-extended, electron-rich, conjugated systems providing desirable properties for applications discussed herein. In solution, DHPPs absorb in the ultraviolet (UV) region of the electromagnetic spectrum (EMS) and display vibrant colors in oxidized states.

The present disclosure relates to a variety of DHPP monomeric units and synthetic processes thereof. The present disclosure relates to the ability to use the electronic character of functional substitutions along the 2,5-positions of a DHPP scaffold to create a number of DHPPs. This allows manipulation of the radical cation to achieve color control of DHPP-based monomers.

The present disclosure relates to structure-property characteristics of various DHPPs. Structure-property characteristics are explored by synthetically altering functional groups on DHPP scaffolds. Functional groups can be electron-withdrawing, electron neutral, or electron-donating. Functional groups can be substituted at ortho-, meta-, and para-positions. Such various alterations and substitutions display the tunability and color control of DHPPs. In such an example, a color-controlled molecule is a molecule that can be selectively designed to exhibit specific colors. Such control may be accomplished by selecting a molecular scaffold. Such control may further be accomplished by coupling one or more end groups to the molecular scaffold. Such end groups may be further substituted to achieve different colors. A molecular scaffold coupled to one or more end groups may be referred to as an electrochrome. Electrochrome colors may further be controlled by subjecting said scaffold and end group compositions to various environments. In a non-limiting example, various environments can be selected by increasing or decreasing the amount of oxidant in an environment. By controlling properties of DHPPs, DHPP-based copolymers can be selectively designed for electrochromic applications.

The present disclosure relates to a color-controlled family of anodically coloring DHPP electrochromic monomers. The present disclosure relates to synthetic processes of such monomers. Synthetic processes may include synthesis of reagents including but not limited to alkylated aldehydes. Synthetic processes include but are not limited to synthetic schemes to create DHPP functionalized molecules and brominated or fluorinated monomers.

The present disclosure relates to a variety of tests to confirm identities of synthesized DHPPs and characteristics thereof. Absorbance spectra and ¹H and ¹³C NMR are used to test resultant DHPPs to confirm synthetic products. This demonstrates that theoretical products were experimentally produced. Electrochemical studies show redox properties of compositions described herein using cyclic voltammetry and differential pulse voltammetry. Solution oxidation studies show changes in optical properties with increasing oxidation via titration. Color coordinate data is collected during solution oxidation.

The present disclosure relates to the use of a number of DHPPs in polymers. Polymers include copolymers. The present disclosure relates to at least two DHPP-containing copolymers. DHPP copolymers provide for ease of solution processing of anodically coloring electrochromes. The disclosure provides the first example of a high-contrast, solution-processable, anodically-coloring DHPP-based copolymer.

The present disclosure relates to DHPP-containing copolymers with controllable optical properties. Optical properties may be controlled by manipulating electronic characteristics of the 2,5-positions of DHPP monomers incorporated into polymers. Incorporation of DHPP variants into copolymers provides transmissive-to-color electrochromic polymers. DHPP provides the added advantage of synthetic simplicity when synthesizing copolymers compared to existing polymer building blocks that are synthetically complex. As an example, DHPPs can be synthesized in one step, in air, and without column chromatography purification.

In an example, the present disclosure relates to the synthesis of various DHPP-co-Thiophene copolymers. Copolymers may be synthesized using Suzuki polycondensations with optimized reaction conditions.

The present disclosure relates to polymeric electrochromes that improve on existing polymeric electrochromes. In an example, viologen-based electrochromes tend to undergo dimerization side reactions that lead to decreased reversibility and operational stability with repeated cycling. In an additional example, ProDOT polymers, like other cathodically coloring polymers, exhibit a decreased overall contrast due to residual red-light absorbance in their oxidized state.

The present disclosure relates to studies of optical properties of copolymers discussed herein. Optical properties may be studied by comparing calculated and experimental absorbance spectra. Copolymers also are subject to solution oxidation studies and colorimetry analysis of copolymer solutions.

The present disclosure additionally relates to the use of monomers and copolymers disclosed herein in various applications. Applications include, as a non-limiting example, a variety of electrochromic films. The present disclosure demonstrates electrochemistry of DHPP polymer films, including thin films, using cyclic voltammetry and differential pulse voltammetry. Examples of films are provided including processing and switching relating to such films. Such example films demonstrate the ability to control the color of films by incorporating monomers and polymers described herein. Additionally, such films demonstrate the ability to process monomers and polymers into films and the stability of such films.

Conjugated polymers are traditionally synthesized with targeted properties in mind while sustainable considerations or green chemistry principles can be overlooked. As a means to minimize waste, reduce the number of synthetic steps, and use safer reagents, pyrrolo[3,2-b]pyrroles (DHPPs) are explored as novel building blocks for synthesizing conjugated polymers. The present disclosure relates to DHPPs and related methods of synthesis. Such synthetic methods may utilize only one step compared to multiple preparatory steps required to synthesize building blocks to attain monomers useful for polymerizations according to previous methods. DHPPs synthesized using reduced complexity synthesis, as described herein, are capable of achieving robust color-control. DHPPs are excellent candidates for this because monomers are accessible in one synthetic step from commercially relevant starting materials (anilines and aldehydes), monomer synthesis is performed in air, and purification does not require chromatography. Dihalogenated DHPP comonomers are easily synthesized and readily participate in metal-catalyzed cross-coupling reactions and polymerizations to obtain a family of highly tailorable chromophores and polymers. Fundamental design-structure-property relationships are established by studying synthesized polymers with optical, electrochemical, and thermal characterization techniques. Beyond fundamental studies, this family of polymers demonstrates their utility in redox and solid-state applications, evident by functioning as high-contrast electrochromes or active-layer material in organic photovoltaics. The synthetic complexity of resulting polymers also is determined and utilizing DHPPs yields alternating copolymers with synthetic complexities lower than more commonly studied analogues.

Outcomes from this work provide a simpler approach for synthesizing conjugated polymers while maintaining tailorability and functionality in device-inspired measurements and motivates further utilization of electron-rich pyrrolopyrroles in organic electronics.

### III. Molecular Scaffolds and End Groups as Chromophores

The present disclosure relates to electron-rich pyrrolopyrroles and their applications in a variety of optoelectronic applications. Such pyrrolopyrroles include synthetic simplicity and tailorability that are advantageous for synthesizing structurally-diverse scaffolds.

The presence disclosure relates to the influence of peripheral functionality on optoelectronic properties of conjugated materials. Such understanding allows development of chromophores for myriad applications. As a non-limiting example, π-extended 1,4-dihydropyrrolo[3,2-b]pyrrole (DHPP) chromophores with varying electron-donating or electron-withdrawing capabilities may be synthesized. Such synthesis may include Suzuki cross-coupling reactions.

The present disclosure relates to methods of measuring the influence of functionality (e.g., various end groups) on optoelectronic properties of DHPP chromophores. As a non-limiting example, chromophores display differences in the UV-vis absorbance spectra measured via UV-vis absorbance spectroscopy in addition to changes in the onsets of oxidation measured with cyclic voltammetry (CV) and differential pulse voltammetry (DPV). As an additional, non-limiting example, the present disclosure relates to solution oxidation studies showing that variations in the electron-donating and -withdrawing capabilities of groups attached to a DHPP chromophore result in different absorbance profiles of the radical cations that correspond to quantifiably different colors. As such, the present disclosure provides fundamental insights into the molecular design of DHPP chromophores and their optoelectronic properties.

The present disclosure relates to the ability to tune the optoelectronic properties of DHPP chromophores in their neutral and oxidized states by modifying end groups attached to a DHPP scaffold. The present disclosure provides an enhanced the understanding of structure-property relationships to allow creation of DHPP-based materials.

The present disclosure relates to chromophores displaying high-contrast electrochromism capable of use in electronic devices. The present disclosure relates to the use of discrete in organic photovoltaic (OPV), organic light-emitting diode (OLED), redox, and bioimaging applications.

The present disclosure relates to discrete, π-conjugated chromophores. A discrete chromophore may be one that does not occur in a chain of chromophores including but not limited to a monomeric unit. Such chromophores allow studies of structure-property relationships with enhanced ease compared to polymers due to the ease of introducing subtle structural changes on a molecular level. Control over the structure of a discrete chromophore provides analysis of a single molecular system versus a complex, polydisperse polymer sample. The present disclosure thus relates to an advantageous, accurate elucidation of optoelectronic properties of chromophores. Such properties are displayed through minimal changes in functionality of chromophores that would be more convoluted if studied in polymers. Such functionality may refer to coupling various end groups to a DHPP scaffold or substituting end groups already coupled to such a DHPP scaffold.

The present disclosure relates to the use of one or more molecular scaffolds. One molecular scaffold that has emerged as a useful building block for conjugated materials is 1,4-dihydropyrrolo[3,2-b]pyrrole (DHPP). Molecular scaffolds may also be referred to as molecular cores. Such scaffolds can be coupled to one or more end groups. In such an example, the one or more end groups attached to a molecular core may be substituted to provide the resulting chromophore with various selectable characteristics. One molecular scaffold that has emerged as a useful building block for conjugated materials is 1,4-dihydropyrrolo[3,2-b]pyrrole (DHPP). In such examples, a chromophore may include a molecular scaffold, including but not limited to DHPP and derivatives thereof, and one or more end groups coupled to the scaffold. DHPP and derivates thereof allow for simple synthesis, ease of purification, and expansive design spaces on the periphery of the DHPP scaffold by selecting certain starting materials. DHPPs are electron-rich scaffolds that may be used in applications such as photocatalysis and as active-layer materials in organic resistive memory (ORM) devices, organic field-effect transistors (OFETs), and OPVs. DHPPs may be used in advanced optoelectronic materials. The present disclosure provides an enhanced understanding of structure-property relationships of DHPP chromophores.

Synthetic simplicity, structural tailorabilty, and expansive applicability of DHPPs motivated previous work to synthesize a “synthetically simple” DHPP-containing copolymer. Bell, K.-J. J., et al., Chem. Mater., 34: 8729-8739 (2022). The resulting polymer was solution processible and displayed yellow-to-black electrochromism with an applied electrochemical potential. This demonstrated that DHPP-based materials could potentially be useful as multi-colored electrochromes. However, for DHPPs to realize such utility in electrochromic applications, it would be necessary to establish structure-property relationships that relate the choice of comonomers to optical, redox, and color properties of both neutral and oxidized species.

The present disclosure relates to a structural diversity of DHPPs. Such diversity allows DHPP and its derivatives to be readily manipulated. Such manipulation may occur, as a non-limiting example, by selecting various anilines and aldehydes that participate in an Fe-catalyzed reaction used for synthesizing DHPPs. π-extended DHPP systems synthesized via metal-catalyzed cross-coupling reactions are less prevalent in the art.

The present disclosure relates to subtle structural changes of molecular coupling partners to manipulate optoelectronic properties of π-extended DHPPs as neutral and oxidized molecules. The present disclosure relates to a variety of π-extended DHPPs designed based on differences in the electron-donating or -withdrawing capabilities of peripheral substituents (e.g., end groups). Such DHPPs were modeled with time-dependent density functional theory calculations. After predicting differences in the optical properties of such DHPP chromphores, the targeted molecules were synthesized. Synthesis included Suzuki cross-coupling reactions. As a non-limiting example, Pd-catalyzed Suzuki cross-coupling reactions between a dibrominated DHPP and corresponding boronic acid coupling partner may be used. Experimental characterizations of the optoelectronic properties of the synthesized DHPP chromophores aligned with the trends predicted theoretically.

The present disclosure relates to a variety of peripheral substituents. Peripheral substituents may also be referred to interchangeably as end groups. Such substituents ranged from electron-withdrawing to electron-donating substituents. With increasing electron-donating character of such substituents, the UV-vis absorbance shifted from the visible to UV region of the electromagnetic spectrum, and the onsets of oxidation were lowered as measured by cyclic voltammetry (CV) and differential pulse voltammetry (DPV).

The present disclosure relates to chemical oxidation of DHPP chromophores described herein. Upon such oxidation, most of the chromophores transitioned from the UV region of the electromagnetic spectrum (EMS) into the visible region to achieve distinctly different absorbance profiles.

The present disclosure relates to colorimetry analysis of DHPP chromophores. The color profiles of the various DHPP chromophores containing different peripheral substituents were found to be quantifiably different. As a non-limiting example, at least two of the chromophores display high-contrast color changes. In such an example, the at least two π-extended DHPPs display transmissive-to-colored transitions that demonstrate the first examples of DHPPs potential utility as high-contrast anodically coloring electrochromes. Such color changes are advantageous for anodically-coloring electrochromism applications. DHPPs as high-contrast anodically coloring electrochromes according to the present disclosure are capable of being made in a single synthetic step. The present disclosure relates to modular manipulations on the periphery of DHPP chromophores and the resulting influence on properties applicable to redox-active applications, including electrochromism. The present disclosure relates to DHPP chromophores useful for a variety of applications.

Electrochemical studies reveal the π-extended DHPPs possess relatively low oxidation potentials (of about 0.4 V-0.6 V vs. Ag/AgCl) which may render them useful in redox-active applications, such as electrochromics. The resulting optical properties of neutral and oxidized chromophores were studied via UV-vis absorbance spectroscopy and reveal a dependence between the substitution patterns and functionality on absorbance characteristics. Combined, these results provide strategies for tuning the optoelectronic properties of DHPP molecules.

The present disclosure relates to a variety of proven functionalization strategies of DHPPs. Such functionalization allows tunability of DHPP chromophores and their optical properties. The present disclosure relates to an ability to participate in cross-coupling reactions of the various DHPP chromophores disclosed herein. Such ability provides applicability of the DHPP chromophores in optoelectronic applications including high-contrast electrochromism as molecules or polymers.

The present disclosure relates to electron-rich pyrrolopyrroles. The present disclosure relates to numerous optoelectronic applications. The present disclosure relates to enabling a thorough understanding of how functionalities of DHPPs influence application-inspired properties. The present disclosure hypothesized a family of π-extended DHPP chromophores to lend insight into the effects of structural variations, specifically changes in the electron-donating and electron-withdrawing character, on optical properties of neutral and oxidized chromophores. Chromophores may be accessed through robust, high-yielding Pd-catalyzed Suzuki cross-coupling reactions between a dibrominated DHPP and the corresponding boronic acid coupling partner. The resulting optical properties of neutral and oxidized chromophores were studied via UV-vis absorbance spectroscopy and revealed a dependence between the substitution patterns and functionality on the absorbance characteristics. Specifically, as the electron-donating nature of the peripheral substituent is increased, there is an observed blue-shift in the absorbance of neutral molecules and a red-shift in spectra measured for chemically oxidized chromophores. These results were further confirmed via TDDFT, and the agreement between theory and experiment opens the opportunity for theory-guided DHPP-containing material. Electrochemical studies also confirm substituent effects influence redox properties and that the π-extended DHPPs possess relatively low oxidation potentials (˜0.4-0.6 V vs Ag/AgCl). The low onsets of oxidation may render the DHPPs useful in redox-active applications and motivated the study of chromophores as electrochromes. Notably, two π-extended DHPPs display transmissive-to-colored transitions upon oxidation and demonstrate the potential utility of DHPPs as high-contrast anodically coloring electrochromes. Combined, these results provide strategies for tuning the optoelectronic properties of DHPP molecules and expanding associated utility as materials used in electrochemical applications. The large number of verified functionalization strategies of DHPPs and associated ability to participate in cross-coupling reactions suggest DHPP chromophores may find applicability in optoelectronic applications such as high-contrast electrochromism as molecules or polymers.

### IV. Anodically Coloring Electrochromes

The present disclosure relates to anodically coloring electrochromes. Such anodically coloring electrochromes include discrete anodically coloring electrochromes. Such anodically coloring electrochromes are capable of being used as high-contrast alternatives to cathodically coloring electrochromes. Anodically coloring electrochromes may offer enhanced optical contrast during electrochemical switching protocols compared to cathodically coloring alternatives.

The present disclosure relates to color control of anodically coloring electrochromes by manipulating the position and profile of the radical cation absorbance while also being synthetically accessible for generating polymeric material.

The present disclosure relates to the use of DHPP and derivatives thereof as an anodically coloring electrochrome. π-extended DHPPs display tailorable optical and redox properties, as described herein. Such DHPPs also provide simplified synthesis of anodically coloring electrochromes due to the synthetic simplicity of DHPP itself. The variety of DHPP chromophores with varying functionalities described herein are high-contrast chromophores.

The present disclosure relates to a variety of DHPP chromophoric molecules. Such molecules in solution absorb in the ultraviolet region of the electromagnetic spectrum and display various vibrant colors in their oxidized states.

The present disclosure relates to the ability to use the electronic character along the 2,5-positions of a DHPP scaffold to manipulate the radical cation and achieve color control of DHPP-based electrochromes. As such, the present disclosure relates to the first DHPP molecules that are color-controlled, high-contrast electrochromes for applications within anodically coloring electrochromics.

The present disclosure relates to organic electrochromic materials. Such materials may be small molecules or polymers and give rise to expansive design spaces. The present disclosure relates to variety of novel electrochromes. Organic electrochromes include cathodically coloring electrochromes (CCEs) and anodically coloring electrochromes (ACEs). The present disclosure relates in particular to ACEs. Organic electrochromes display redox activity and are capable of forming a radical species. Redox activity and the ability to form a radical species both require conjugated materials.

Examples of conjugated materials include 1,1′-disubstituted 4,4′-bipyridinium salts, known as viologens, and dioxythiophenes (DOTs). Viologens include molecular and polymeric systems that form radical cations that are relatively stable and demonstrate high-contrast electrochromic properties through transmissive to colored reduction transitions. Viologens, however, tend to undergo dimerization side reactions that lead to decreased reversibility and operational stability with repeated cycling. An additional example includes ProDOTs. ProDOTs are used as polymeric materials that exhibit cathodic transitions from visible spectrum to the infrared region, have fast response times, and are highly conductive. However, ProDOT polymers, like other cathodically coloring polymers, exhibit a decreased overall contrast due to residual red-light absorbance in their oxidized state.

The present disclosure relates to anodically coloring electrochromes. Unlike the reduced contrast, including decreased film contrast leading to poor coloration of films, of current cathodically coloring electrochromes, anodically coloring materials display high-contrast switching. Anodically coloring electrochromic molecules include various functionalized dioxythiophenes. Anodically coloring electrochromes are capable of manipulating the radical cation absorbance profiles through structural variations. Optoelectronic properties of electrochromic materials may be controlled by changing functionality, primarily through use of electron-donating substituents. The present disclosure relates to the use of the electron-rich nature of molecules to influence optoelectronic properties. The present disclosure relates to varying electronic character design motifs to achieve a complete color palette of anodically coloring electrochromes.

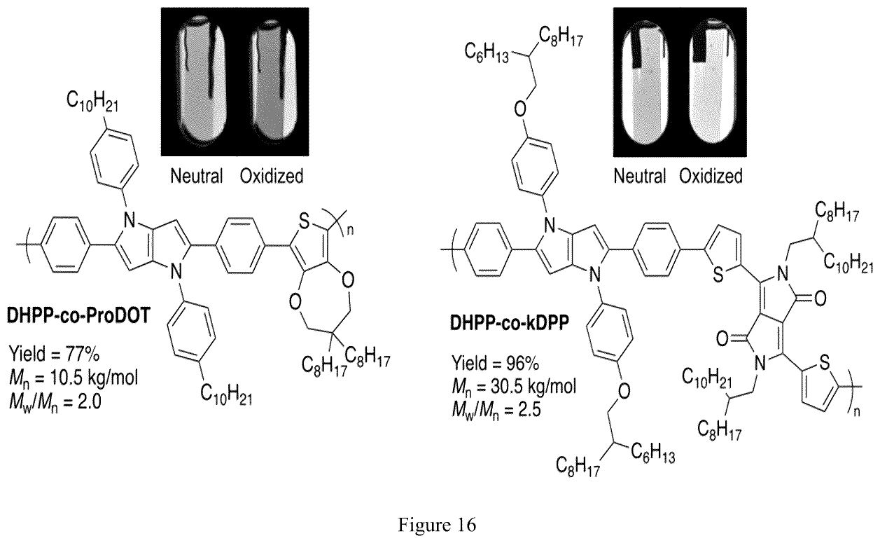

As a non-limiting example, the present disclosure relates to a design motif including dihydropyrrolo[3,2-b]pyrrole (DHPP). The present disclosure relates to uses of DHPP in anodically coloring electrochromes. DHPP provides synthetic simplicity, ease of functionality, and tunable optoelectronic properties. DHPPs are synthesized in one synthetic step, in air, and typically do not require column chromatography for purification. DHPPs are electron-rich systems. Such systems are advantageous for redox applications due to low onsets of oxidation. At least two DHPP-containing copolymers display utility as electrochromic materials. Bell, K.-J. J., et al., Chem. Mater., 34: 8729-8739 (2022); Bell, K.-J. J., et al., Macromolecules (2023). Bell et al. synthesized two different polymers, DHPP-co-ProDOT and DHPP-co-kDPP, that displayed multi-colored or color-to-transmissive electrochromic properties based on the choice of the comonomer (FIG. 16). DHPPs display a number of advantages. Advantages include but are not limited to synthetic simplicity of DHPPs, facile tunability of DHPP properties through structural modifications, and electroactivity/electrochromism. The present disclosure provides an understanding of structure-property relationships of DHPPs and feasibility of DHPPs as electrochromic materials. FIG. 16 shows examples of pyrrolopyrroles as electrochromic materials. Structures adapted from Bell and coworkers. Bell, K.-J. J., et al., Chem. Mater., 34: 8729-8739 (2022); Bell, K.-J. J., et al., Macromolecules (2023).

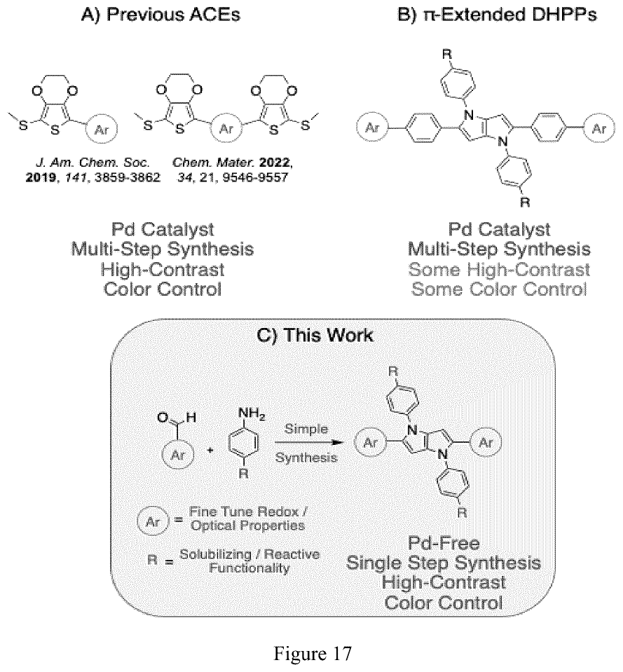

The present disclosure relates to the anodically coloring optoelectronic properties of π-extended DHPPs and their use as novel anodically coloring electrochromes (FIG. 17). The present disclosure relates to synthetic schemes using functionalized anilines and aldehydes to produce molecules and monomers with tailorable properties. The present disclosure provides understanding of structure-property relationships of molecular DHPPs. This understanding gives rise to a novel class of anodically coloring electrochromes. Such electrochromes are capable of being used in polymeric materials. Manipulation of the radical cations of DHPPs is achieved by the incorporation of electron-withdrawing, -neutral, and -donating substituents. This manipulation allows the realization of a color-controlled family of anodically coloring DHPP electrochromes. DHPP's characteristics including but not limited to tailorability, synthetic simplicity, and the like are advantageous when manipulating the radical cation of molecules while maintaining optical and electrochemical standards useful for various applications. FIG. 17 shows representative structures of previous ACE molecules, previous π-extended DHPPs, and molecules according to the present disclosure. Such structures use simplified DHPP molecules to achieve high-contrast ACE systems.

The present disclosure relates to the synthesis of discrete molecules, including ACEs, to attain systems that are amenable to being incorporated into films. The present disclosure relates to enhanced solution-processability of ACE materials while attaining simple, high-contrast organic electrochromes. The present disclosure relates to the use of DHPPs to attain a family of ACE molecules. Such a family of ACE molecules relating to DHPP is capable of being used to create DHPP copolymers with desired properties through tunability. The present disclosure relates to the first example of color-controlled DHPP chromophores attained in one synthetic step. The present disclosure relates to a synthetically simple, high-contrast electrochromes of a variety of colors. The present disclosure relates to influences of substituents on properties of electrochromes. Such properties can be measured, as non-limiting examples, by electrochemistry, optical absorbance, and the like. The present disclosure relates to novel design motifs for high-contrast, color-controlled DHPP-based electrochromes.

### EXAMPLES

### Example 1

DHPP chromophores have been previously studied, but DHPPs coupled with aromatic substituents of varying electronic character are less prominent. To understand how these structural characteristics influenced optical properties, such as the absorbance of neutral and oxidized chromophores, a family of π-extended DHPPs with methyl (DHPP 3), methoxy (DHPP 4), trifluoromethyl (DHPP 5), and cyano substituents (DHPP 6) were chosen and modeled using time-dependent density functional theory (TDDFT).

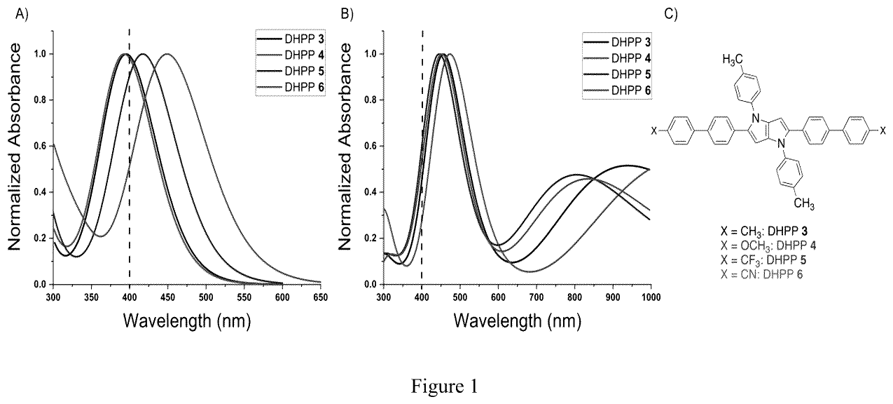

TDDFT is a tool for elucidating the optical properties of conjugated molecules and can been used to develop structure-property relationships of molecules. In the present disclosure, the B3LYP-631G* functional/basis set was used to determine the optical properties of the π-extended DHPPs. The calculated neutral UV-vis absorbance spectra predicted that DHPP 5 and 6 would absorb in the visible region while DHPP 3 and 4 would partially absorb in the UV (FIG. 1A). According to the calculated spectra, DHPPs 3 and 4 had the same relative absorbance maximums (λₘₐₓ) of about 395 nm and 393 nm, signifying that changing from an electron-neutral substituent to an electron-donating moiety did not significantly impact the optical properties of the neutral molecules. However, going from DHPP 5 to DHPP 6, showed a red-shift in the λₘₐₓ from about 418 nm to about 449 nm, suggesting that increasing the electron-withdrawing strength resulted in a decrease in the energy gap between the highest occupied molecular orbital (HOMO) and lowest unoccupied molecular orbital (LUMO) relative to DHPP 5. The reduction in the energy gap was attributed to the push-pull effect from the electron-rich DHPP backbone to the electron-deficient pendant.

To understand optical properties pertinent to electrochromic applications, TDDFT calculations were used to predict substituent effects on the absorbance characteristics of radical cations. Upon removal of an electron, the optical transitions shifted to lower energies across the visible spectrum (FIG. 1). Each molecule possessed two absorbance features corresponding to transitions from singly occupied molecular orbitals (SOMO) to the LUMO. The high-energy absorption SOMO-α→LUMO-α of the radical cations with absorbance maxima (λₘₐₓ^{α}) ranging from about 430 to about 475 nm provided the first insight into the ability to manipulate the position of the radical cation.

Turning to long-wavelength absorption of the radical cations, or the SOMO-β→LUMO-β, substituent effects were much more pronounced. As shown in FIG. 1B, the absorbance max of the SOMO-β→LUMO-β(λₘₐₓ^{β}) for DHPP 3 was about 940 nm, DHPP 4 greater than about 1000 nm, DHPP 5 of about 810 nm, and DHPP 6 of about 840 nm. The differences in the SOMO-β were emphasized by the red shift in absorbance between DHPP 3 to DHPP 4 and showed that increasing the electron-donating character of peripheral substituents shifted the (λₘₐₓ^{β}) into the NIR region. Alternatively, going from electron-donating to electron-withdrawing, the SOMO-β experienced a blue-shift illustrated by the variation in absorbance of DHPP 4 to DHPP 6. These calculations were provided visualization of substituent effects on radical cation absorbance of DHPP chromophores and motivated the continued investigation into the structure-property relationships of DHPPs as neutral and oxidized species.

FIG. 1 shows TDDFT UV-vis absorbance spectra for π-extended DHPPs 3-6 with varying functionalities as (FIG. 1A) neutral and (FIG. 1B) oxidized chromophores. FIG. 1C displays representative DHPP structures used for TDDFT calculations. R-groups are truncated to methyl substituents for simplicity during the calculations.

### Example 2

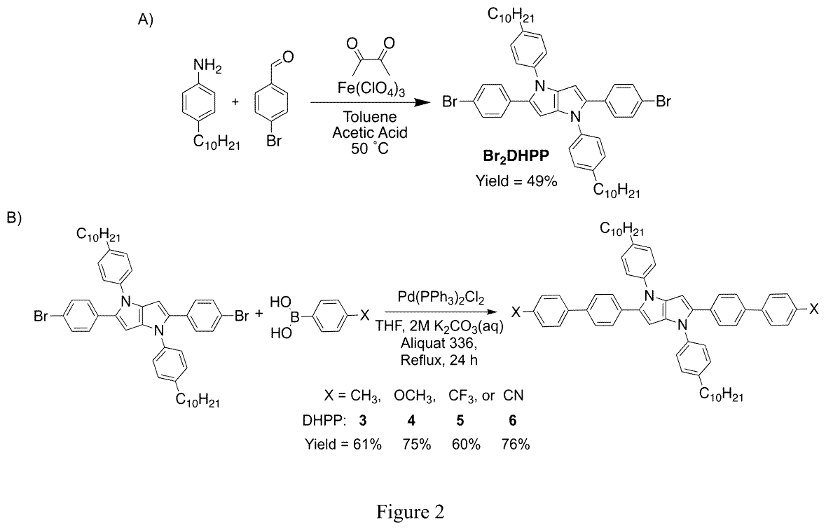

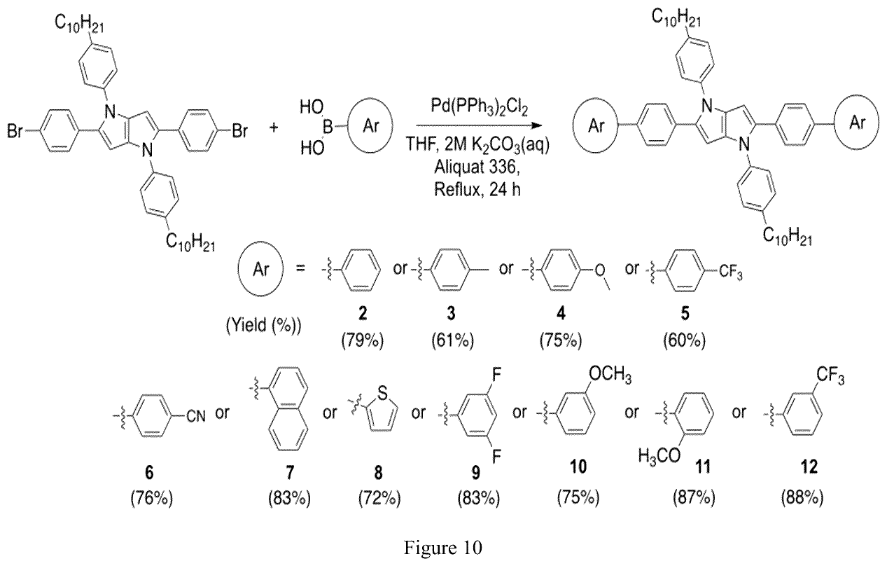

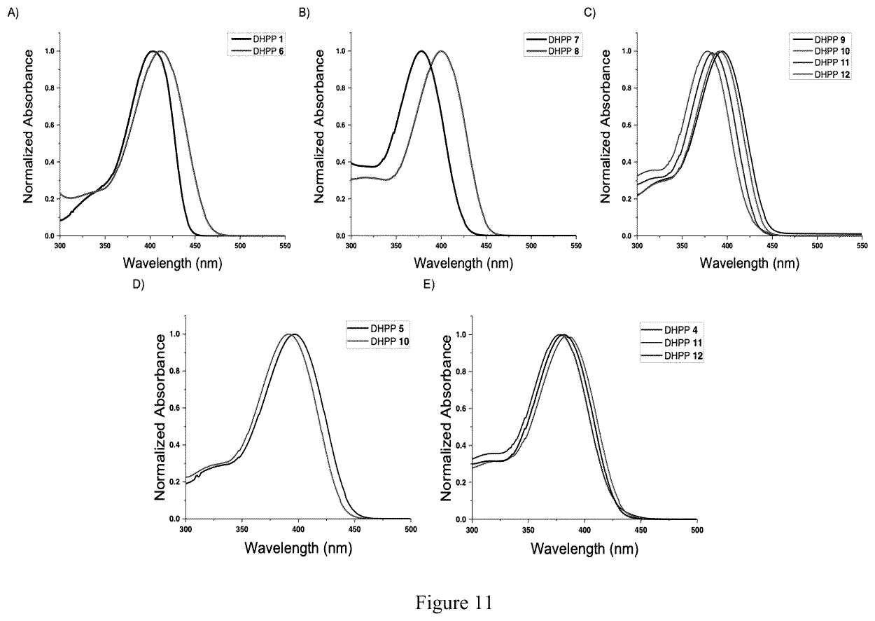

After showing that the optical properties would vary in theory based on substituent choice, a family of π-extended DHPP chromophores was synthesized. The halogenated starting material Br₂DHPP was obtained via the Fe-catalyzed multicomponent reaction procedure adopted by Bell et al. and was used in Suzuki cross-coupling reactions (FIG. 2A). Bell, K.-J. J., et al., Chem. Mater., 34: 8729-8739 (2022). These Suzuki reactions reacted the dibrominated DHPP with boronic acid cross-coupling partners in tetrahydrofuran (TIF) using bis(triphenylphosphine)palladium(II) dichloride (Pd(PPh₃)₂Cl₂) as the palladium source (FIG. 2B). The para-substituted chromophores modeled with TDDFT, DHPPs 3-6, were synthesized in addition to chromophores with substituents at various positions around the phenyl ring or alternative aromatics (DHPPs 7-12) (as shown in FIG. 10). After workup and purification, the π-extended DHPPs were obtained in moderate-to-high yields that were consistent with previous reports involving Pd-catalyzed reactions of DHPPs. The structure and purity of the DHPPs were confirmed via ¹H and ¹³C NMR in tandem with melting point experiments, all of which is shown in FIGS. 11-34 and Table 1, as shown below.

TABLE 1
Melting point ranges for DHPPs 2-12. The narrow temperature
ranges are consistent with a high level of purity.

| DHPP | Melting Point (° C.) |
|---|---|
| 2 | 172-174 |
| 3 | 169 |
| 4 | 187-189 |
| 5 | 190-192 |
| 6 | 223-225 |
| 7 | 138-141 |
| 8 | 188-189 |
| 9 | 157-159 |
| 10 | 135-141 |
| 11 | 122-124 |
| 12 | 119-121 |

These results represented a robust route to achieving π-extension through the 2,5-positions of DHPP chromophores for an additional strategy for tailoring DHPP dyes.

FIG. 2 shows (FIG. 2A) synthesis of Br₂DHPP via the Fe(III)-catalyzed multicomponent reaction using protocols from Tasior and coworkers and (FIG. 2B) synthesis of π-extended DHPP chromophores with various electronic character via Suzuki cross-coupling reactions and their corresponding yields. Tasior, M., et al., Chem. Eur. J., 25 (2): 598-608 (2019).

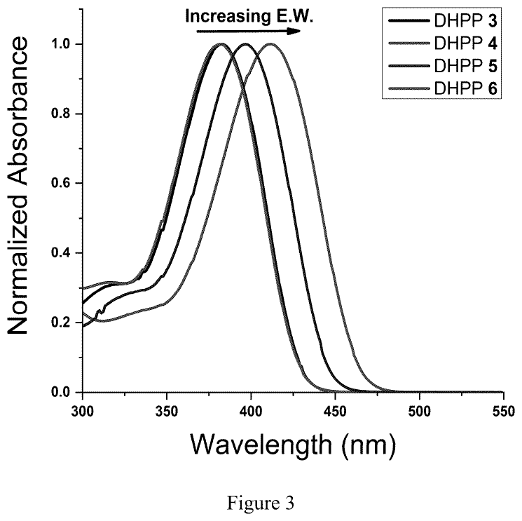

Once structure and purity were confirmed, the UV-vis absorbance spectra of all the DHPPs were measured. First and foremost, the original DHPPs used in TDDFT were found to follow the same trends seen within the calculated spectra (FIG. 3 and Table 2 below).

TABLE 2
UV-vis absorbance maxima for the neutral
π-extended DHPP chromophores.

| Chromophore | (λₘₐₓ^{neu}) (nm) |
|---|---|
| 1 | 405 |
| 2 | 383 |
| 3 | 383 |
| 4 | 382 |
| 5 | 397 |
| 6 | 412 |
| 7 | 379 |
| 8 | 400 |
| 9 | 394 |
| 10 | 391 |
| 11 | 383 |
| 12 | 378 |

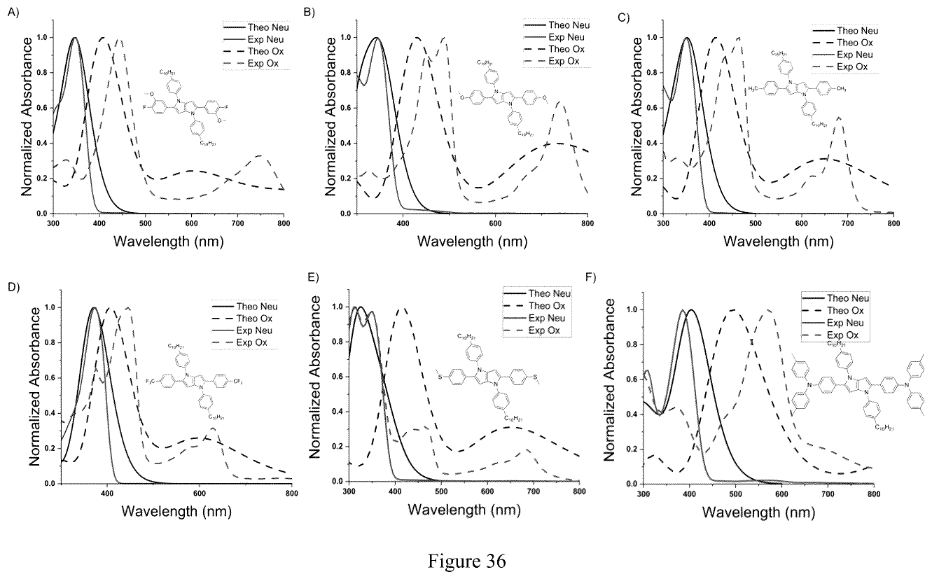

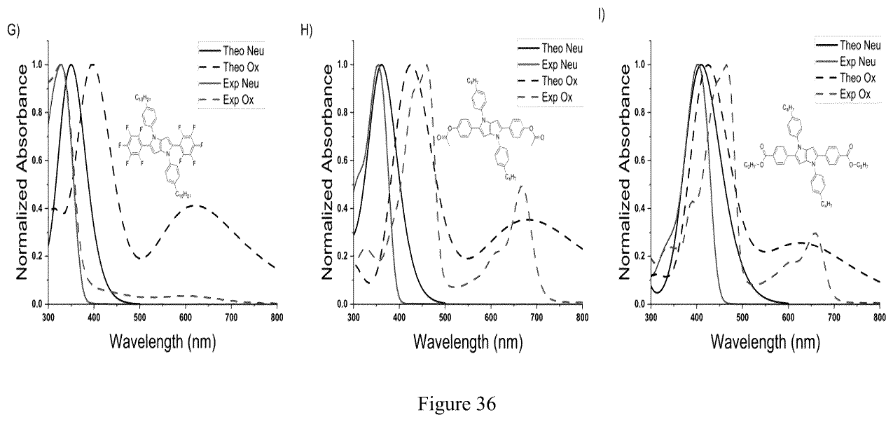

Specifically, DHPP 3 and 4 had similar absorbance maxima (λₘₐₓ) values of about 383 nm and 382 nm, while DHPP 5 was red shifted compared to those two DHPPs at about 397 nm. DHPP 6 exhibited a further red shift to 412 nm, consistent with predicted spectra. The precision between theory and experimental confirmed that the level of theory was adequate for modeling the optical properties of π-extended DHPPs. Notably, DHPPs 3, 4, and 5 mostly absorbed within the UV region of the EMS, which indicated a diminished push-pull effect with decreasing electron-withdrawing effects and was consistent with the DHPP core being highly electron rich. Results displaying the optical properties of DHPPs 7-12 are shown in FIG. 11 and Table 2. Results showing how increasing the π-conjugation of DHPPs, by comparing DHPP 1 and DHPP 6, are also included FIGS. 36-38 and Tables 3-4 below.

TABLE 3
Electronic properties of DHPP 1 and DHPP 6 obtained from
electrochemical and optical characterizations to understand
the influence of π-conjugation on redox activity.

| | Eₒₙₛₑₜ^{ox} | HOMO | LUMO | E_{gap} |
|---|---|---|---|---|
| Chromophore | (V) | (eV)^{a} | (eV)^{b} | (eV)^{c} |
| 1 | 0.68 | −5.8 | −3.1 | 2.7 |
| 6 | 0.58 | −5.7 | −3.1 | 2.6 |

^{a}Calculated given HOMO = −(Eₒₙₛₑₜ^{ox} + 5.12 eV);
^{b}Calculated from absorbance onset given eV = 1240/λₒₙₛₑₜ + HOMO;
^{c}Calculated from (LUMO − HOMO); all equations are adopted from Cardona and coworkers. Cardona, C. M., et al., Adv. Mater., 23(20): 2367-2371 (2011).

TABLE 4
UV-vis and color coordinate data for DHPP 1 and DHPP
6. The neutral and oxidized λₘₐₓ values correspond
to the SOMO-α → LUMO-α and SOMO-β
→ LUMO-β, while the neutral and oxidized color
coordinates are calculated based on mid-day lighting
standards (D50 illuminant as a 2° observer).

| | | | | Color Coordinates | |
|---|---|---|---|---|---|
| | λₙₑᵤ^{max} | λₒₓ^{max} (nm) | | Neu. | Ox. |
| Chromophore | (nm) | SOMO-α | SOMO-β | (L*a*b*) | (L*a*b*) |
| 1 | 405 | 465 | 655 | 100, | 93, |
| | | | | −5, 12 | −14, 39 |
| 6 | 412 | 490 | 730 | 100, | 83, |
| | | | | −11, 37 | 10, 52 |

Changes in optoelectronic properties with increasing conjugation, such as red-shifted absorbance and lowered oxidation potentials, were observed when comparing DHPP 1 to DHPP 6. Minimal changes to peripheral substituents were shown to influence optoelectronic properties of π-extended DHPPs. Also investigated were the effects of para-functionalized aromatics with differing electronic effects.

FIG. 3 shows UV-vis absorbance spectra for π-extended DHPP chromophores with various functionalities at the para-position of the aromatic coupling partner. Absorbance experiments involved measuring DHPP solutions with concentrations of about 10 mM in toluene while scanning from 300-800 nm. The experimental data was in accordance with the calculated spectra and showed the effect of different functionalities on the absorbance of π-extended DHPP chromophores.

### Example 3

Turning to substitution effects on the redox response of π-extended DHPPs, cyclic voltammetry (CV) and differential pulse voltammetry (DPV) were used to measure onsets of oxidation and observe the reversibility of para-functionalized DHPPs. Electrochemical measurements were performed under an Ar atmosphere in DCM with about 0.5 M tetrabutylammonium hexafluorophosphate (TBAPF₆) as the supporting electrolyte. A Pt wire was used as the counter electrode and Ag/AgCl was used as the reference electrode. For three of the four DHPPs, a glassy carbon electrode was used as the working electrode. For DHPP 6, the exhaustion of material during measurements meant it was necessary to use an optically transparent thin-layer electrochemical (OTTLE) cell with a platinum gauze working electrode. With all choices of working electrode, trends relating to the oxidation potential were extracted.

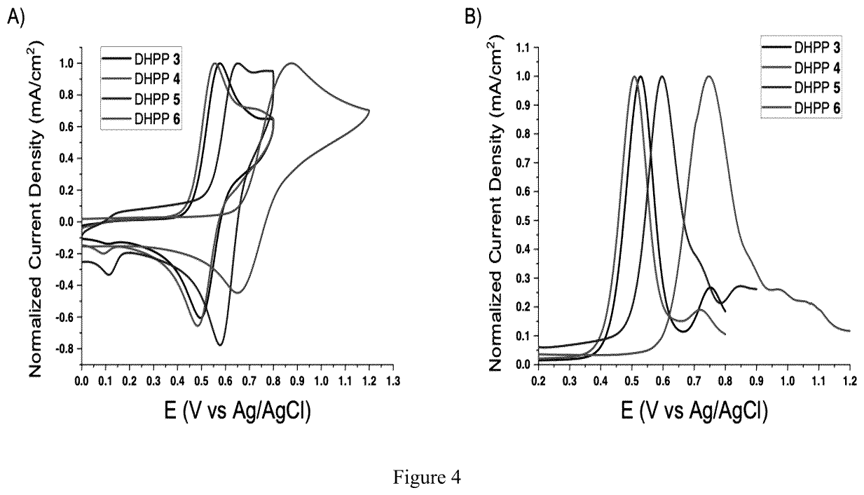

CV and DPV agreed where distinct oxidation peaks were present for the DHPPs (FIG. 4). All four DHPPs displayed distinct oxidations and reductions with relatively low onsets of oxidation. Notably, there was not a significant difference between the onset of oxidation of DHPP 3 and DHPP 4 with onset potentials of about 0.41 V and of about 0.39 V (vs. Ag/AgCl), respectively.

These results demonstrated that an electron-neutral and an electron-donating functionalized π-extended coupling partner exhibited the same relative redox activity due to the electron-rich nature of the DHPP backbone (FIG. 4 and Table 5). These results agreed with the minimal changes measured in UV-vis absorbance experiments.

When comparing DHPP 5 to DHPP 3 and DHPP 4, there was an increase in the onset from about 0.4 V to about 0.51 V (vs. Ag/AgCl) due to the electron-withdrawing nature of the —CF3 functionality in DHPP 5 (FIG. 4 and Table 5). DHPP 6's onset of oxidation was measured to be of about 0.58 V (vs. Ag/AgCl) (Table 5) which is the highest onset of oxidation when compared to the other DHPPs. This was attributed to the high electronegativity of the cyano group and a deepening of the HOMO energy level.

FIG. 4 shows a comparison of redox response of π-extended DHPPs 3-6 via (A) CV and (B) DPV. Measurements were performed using a 0.5 M TBAPF₆/DCM supporting electrolyte and the Ag/AgCl reference electrode (E_{1/2}=0.46 V vs. Fc/Fc⁺).

TABLE 5
Electronic properties of π-extended DHPP chromophores
obtained from electrochemical and optical characterizations.

| | Eₒₙₛₑₜ^{ox} | HOMO | LUMO | E_{gap} |
|---|---|---|---|---|
| Chromophore | (V) | (eV)^{a} | (eV)^{b} | (eV)^{c} |
| 3 | 0.41 | −5.5 | −2.7 | 2.8 |
| 4 | 0.39 | −5.5 | −2.7 | 2.8 |
| 5 | 0.51 | −5.6 | −3.0 | 2.6 |
| 6 | 0.58 | −5.7 | −3.1 | 2.6 |

^{a}Calculated using HOMO = −(Eₒₙₛₑₜ^{ox} + 5.12 eV);
^{b}Calculated from absorbance onset given eV = 1240/λₒₙₛₑₜ + HOMO;
^{c}Calculated using E_{gap} = (LUMO − HOMO); all equations are adopted from Cardona and coworkers. Cardona, C. M., et al., Adv. Mater., 23(20): 2367-2371 (2011).

After determining the onsets of oxidation, the ionization energies (IE), or HOMOs, for each DHPP were estimated. Assumptions that the HOMO could be calculated using the onset of oxidation (Eₒₓ) where the energy of the Fc/Fc⁺ redox couple was about −5.12 eV against the saturated calomel electrode (SCE) energy level versus vacuum were made. The LUMO was estimated by obtaining the onset of absorbance from UV-vis absorbance data. The differences between these values enabled estimation of the energy gap (E_{gap}) for each DHPP (Table 5). The similar energy gaps (about 2.6 to about 2.8 eV) for each DHPP were attributed to the disrupted conjugation between the pyrrolopyrrole backbone and the π-extended substituents due to the pyrrolopyrrole-phenyl dihedral angle (of about 35°) and the phenyl-phenyl dihedral angles (of about 30°). Ultimately, these results demonstrated the effect of the choice of coupling partner on control of the onsets of oxidation through alterations of the electronic character without drastically manipulating the optical band gaps. The above provides an understanding of the redox activity of the DHPPs and influence of substituent groups on optical properties of neutral molecules.

### Example 4

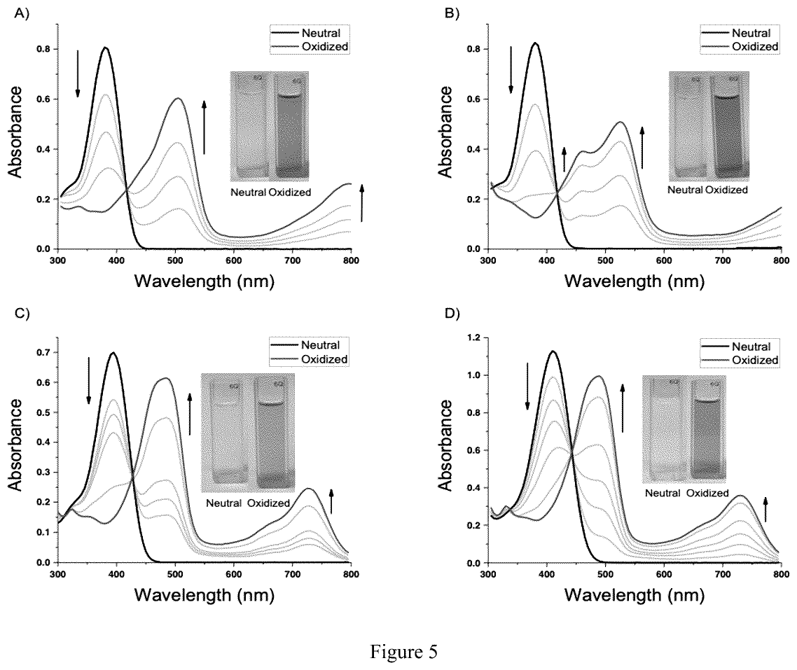

The ability of DHPPs to be chemically oxidized was investigated. Upon oxidation, the changes in absorbance with increasing dopant concentration were monitored with UV-vis absorbance spectroscopy. Solution oxidation experiments were performed for all 4 para-functionalized DHPPs (DHPPs 3-6) by doping the chromophores via titration with an about 0.06 mg/mL Fe(ClO₄)₃·xH₂O solution to elucidate how the substituent functionality of the coupling partner impacted the absorbance of the radical cation. The transition from neutral to oxidized molecules is shown in FIG. 5. Absorbance maxima of the radical cations are reported in Table 6.

Upon chemical doping and formation of the radical cation, DHPPs 3-6 shifted further into the visible region of the EMS and displayed characteristics of SOMO-α→LUMO-α and SOMO-β→-4 LUMO-β transitions. The (λₘₐₓ^{α}) of DHPP 3 was of about 505 nm while DHPP 4 (λₘₐₓ^{α}) of about 525 nm with a distinct shoulder around 460 nm that may be attributed to radical dimerization. The red shift of the cation absorbance from DHPP 3 to DHPP 4 was consistent with the electron-donating capabilities of the —OMe group into the chromophore. DHPP 5 and 6 had similar SOMO-α→LUMO-α transitions with DHPP 5 of about 485 nm and DHPP 6 of about 490 nm. There was a slight blue shift for DHPP 5 compared to DHPP 6 because of reduced electron-withdrawing effects of the —CF₃ group of DHPP 5 compared to the —CN group of DHPP 6. The SOMO-β→LUMO-β transitions for DHPP 5 and 6 were quite similar in profile and positioning, with a (λₘₐₓ^{β}) of about 725 nm for DHPP 5 and of about 730 nm for DHPP 6. Alternatively, the SOMO-β→LUMO-β transitions for DHPP 3 and DHPP 4 were noticeably red-shifted (λₘₐₓ^{β}) greater than about 800 nm) compared to DHPP 5 and 6. The red shift was consistent with increased electron donating capabilities of a methoxy group and implied that increasing the electron donating nature at the para-position of peripheral substituents manipulated the SOMO-β→LUMO-β transition.

These findings allow color control as it relates to application within high-contrast electrochromism. The solution oxidation results supported optical and electrochemical experiments that showed the ability to fine-tune the optoelectronic properties through the choice of functionality on the coupling partner. The overall differences in the absorbance profiles for DHPP 3-6 enabled an investigation into the color profiles of the neutral and radical species to reveal substituent effects on application-inspired properties.

FIG. 5 shows solution oxidation spectra of (FIG. A) DHPP 3, (FIG. B) DHPP 4, (FIG. C) DHPP 5, and (FIG. D) DHPP 6 in DCM using 0.06 mg/mL Fe(ClO₄)₃·xH₂O in ethyl acetate as the dopant. These spectra display changes in the UV-vis absorbance spectra with increasing dopant concentration to elucidate the ability to manipulate the position of the radical cation absorbance.

Table 6 shows UV-vis and color coordinate data for the selected DHPP chromophores. The neutral and oxidized λₘₐₓ values corresponded to the SOMO-α→LUMO-α and SOMO-β→LUMO-β, while the neutral and oxidized color coordinates were calculated based on mid-day lighting standards (D50 illuminant as a 2° observer).

TABLE 6
UV-vis and color coordinate data for the selected DHPP
chromophores. The neutral and oxidized λₘₐₓ values
correspond to the SOMO-α → LUMO-α and SOMO-
β → LUMO-β, while the neutral and oxidized
color coordinates are calculated based on mid-day lighting
standards (D50 illuminant as a 2° observer).

| | | | | Color Coordinates | |
|---|---|---|---|---|---|
| | λₙₑᵤ^{max} | λₒₓ^{max} (nm) | | Neu. | Ox. |
| Chromophore | (nm) | SOMO-α | SOMO-β | (L*a*b*) | (L*a*b*) |
| 3 | 383 | 505 | >800 | 100, | 86, |
| | | | | −1, 3 | 24, 25 |
| 4 | 382 | 525 | >800 | 100, | 81, |
| | | | | −1, 3 | 32, 14 |
| 5 | 397 | 485 | 725 | 100, | 90, |
| | | | | −4, 9 | 3, 42 |
| 6 | 412 | 490 | 730 | 100, | 83, |
| | | | | −11, 37 | 10, 52 |

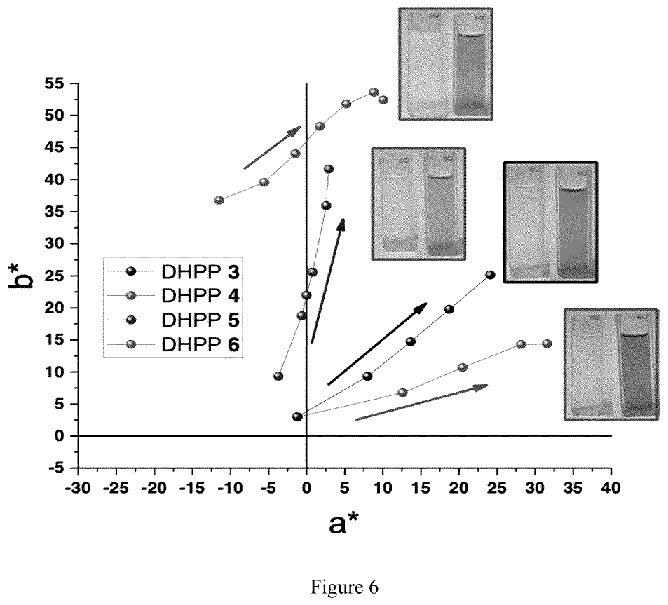

From the UV-vis solution oxidation studies, color coordinate data was extrapolated to track the changes in color of the solution based on the evolving absorbance profiles. The CIE L*a*b* color space was used with a D50 illuminant as a 2° observer. The neutral colors for DHPP 3 and DHPP 4 were found to be similar with L*a*b* of 100, −1, 3 (FIG. 6 and Table 6). The color coordinates corresponded to both molecules being transmissive solutions as neutral molecules that transitioned to colored solutions upon oxidation. Upon the respective color changes, DHPP 3 displayed L*a*b* color coordinates of 86, 24, 25 while DHPP 4 transitioned to L*a*b* values of 81, 32, 14. These color tracks corresponded to both solutions transitioning from transmissive solutions as neutral molecules to red-orange and red solutions, respectively. DHPP 5 also exhibited L*a*b* values consistent with a color-neutral solution (100, −4, 9). Upon oxidation, the color shifted to 90, 3, 42 to yield a vibrant yellow color in solution. Unlike DHPP 3-5, DHPP 6 had neutral color coordinates of 100, −11, 37, which showed a light-yellow color when placed on the coordinate diagram and which was consistent with the red-shifted UV-vis data reported in FIG. 5. Upon oxidation, DHPP 6 displayed a shift in its solution color, resulting in L*a*b* color coordinates of 83, 10, 52 which corresponded to the yellow-to-golden-yellow color change.

All of these results are supported by the photographs presented in FIG. 6. The color data showed that the functionality and choice of coupling partner influenced the radical cation absorbance of DHPP chromophores. By altering the functionality of the coupling partner from electron-donating to electron-withdrawing, the optical and redox properties were manipulated which. This showed the structure-property relationships needed to develop DHPP-containing materials for electrochromic applications.

FIG. 6 shows color coordinates for DHPPs 3, 4, 5, and 6 calculated based on the absorbance change with respect to varying concentrations of chemical dopant. The data illustrates the color control achieved by varying the electronic character through the peripheral substitution of DHPPs. Arrows represent the color track evolving from neutral to oxidized solutions.

### Example 5

The above results provide an understanding of how structural changes influence optoelectronic properties of DHPP-containing molecules. Incorporating these materials into devices is also contemplated. Disclosure below relates to DHPP-based materials functioning in electroactive devices.

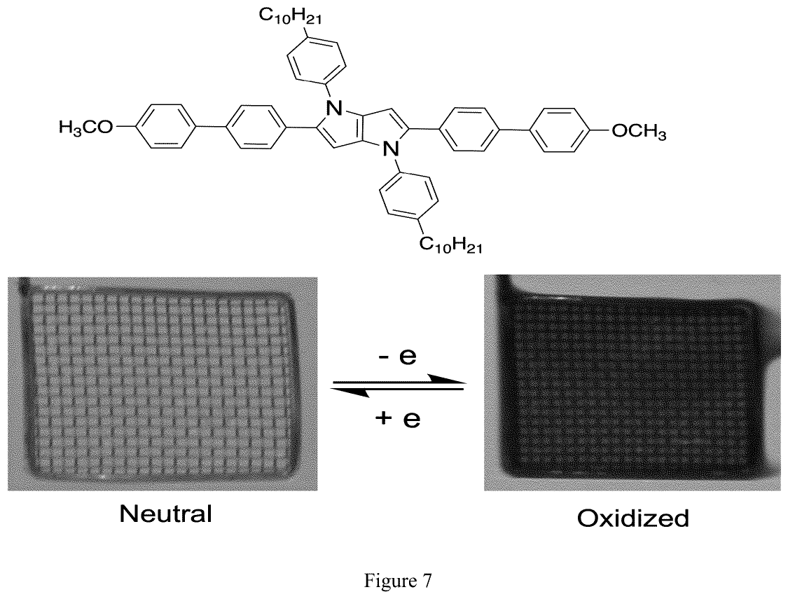

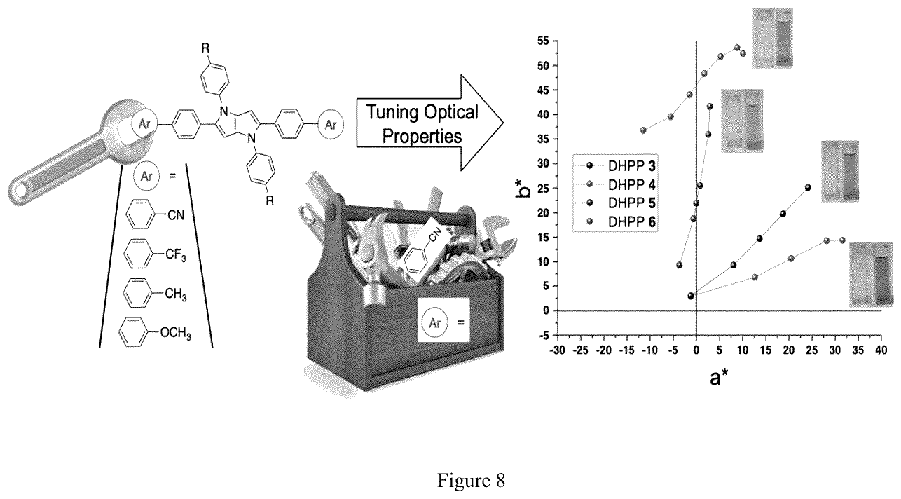

DHPP 3 and DHPP 4 were dissolved in an electrolyte solution and were electrochemically switched between their neutral and oxidized states. Both DHPPs demonstrated high-contrast electrochemical switching from a relatively transmissive neutral state to either orange or red oxidized states (FIGS. 6 and 7). Both of these color transitions were consistent with the solution oxidation studies and the colorimetry data above. These results display the ability to use DHPP chromophores as high-contrast electrochromic materials for applications within organic electronic devices.

FIG. 7 shows electrochemical switching experiments using an OTTLE of the color-controlled, high-contrast DHPP chromophore DHPP 4.

### Example 6

TDDFT calculations using Gaussian 16 and the B3LYP-631G* functional/basis set were performed to elucidate the optical properties of the DHPP molecules synthesized. First, the molecules were constructed in Gaussview, and a geometry optimization was performed to ensure the correct geometry was used in the subsequent calculations. Next, the excited state calculations were run to understand the positioning of the radical cation absorbance. After completion, the data was collected, normalized to the absorbance maximum, and plotted in Origin to report calculated UV-vis absorbance spectra.

All materials used in synthetic protocols were purchased from commercial sources and used as received unless otherwise stated. Anhydrous tetrahydrofuran (THF) dichloromethane (DCM), and toluene were obtained from a Pure Process Technology GC-SPS-7 Glass Contour 800L Solvent Purification System stored under Ar and degassed with argon (Ar) for 15 min. before use.

All column chromatography purifications used 60 Å silica gel (200-400 mesh). ¹H NMR and ¹³C NMR spectra were collected on a Bruker Advance III HD 400 MHz NMR spectrometer with nominal concentrations of 5 mg/mL in CDCl₃. Peaks are referenced to the residual CHCl₃ peak (¹H: δ=7.26 ppm; ¹³C: δ=77.23 ppm).

Melting point ranges were obtained by depositing samples in borosilicate glass capillary tubes before using a DigiMelt MPA 160 instrument to record melting temperatures.

Optical absorbance spectra of solutions with nominal concentrations of 10-20 mM in toluene or DCM for the molecules were acquired using a Varian Cary 60 Scan single-beam UV-vis-near-IR spectrophotometer scanning from 300 to 800 nm.

Solution oxidation experiments involved titrating each solution dropwise with a 0.6 mg/mL Fe(ClO₄)₃·xH₂O solution in ethyl acetate until the radical cation peak reached its maximum absorbance intensity.

Next to the UV-vis absorbance spectra are photographs of neutral and oxidized solutions in quartz cuvettes after adding the maximum amount of oxidant. Photographs are presented without manipulation except for cropping.

Color coordinates were calculated based on the Commission Internationale de l'Eclairage 1976 L*a*b* color standards using a D50 illuminant as a 2° observer.

Cyclic voltammetry (CV) and differential pulse voltammetry (DPV) measurements were performed with a CH Instruments Electrochemical workstation (CHI660D), using a glassy carbon electrode as the working electrode, an Ag/AgCl reference electrode (calibrated versus the Fc/Fc⁺ redox couple, E_{1/2}=46 mV), and a Pt flag as the counter electrode. A 50 mV/s scan rate was used for all electrochemical measurements. An electrolyte solution of 0.5 M tetrabutylammonium hexafluorophosphate (TBAPF₆, 98%) in anhydrous DCM was used for all molecular electrochemical measurements. The cell and working electrode used to measure the CV and DPV data for one chromophore was done using an SEC-C thin-layer quartz glass spectroelectrochemical cell with a platinum gauze working electrode.

Photography was performed in a light booth designed to exclude outside light with controllable LED lighting above providing illumination. A Canon Rebel T7 camera with an 18-55 mm lens was used to capture images. Images are presented without manipulations except for cropping.

Functionalized DHPP-containing molecules were synthesized via an Fe(III)-catalyzed multicomponent reaction.

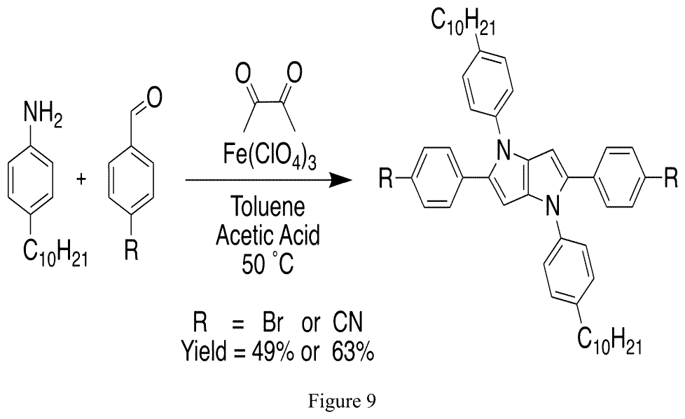

FIG. 9 shows a general synthesis of the brominated DHPP monomer (Br₂DHPP) and DHPP 1 using protocols adopted from Bell et al. Bell, K.-J. J., et al., Chem. Mater., 34: 8729-8739 (2022).

n-decylaniline (8 mmol) and p-bromobenzaldehyde (8 mmol) or 4-cyanobenzaldehyde (8 mmol) were added to a solution of toluene (6 mL) and glacial acetic acid (6 mL) inside a 25 mL round bottom flask equipped with a magnetic stir bar. The reaction mixture was stirred for 1 h in an oil bath set to 50° C. Once the initial heating time was completed, Fe(ClO₄)₃·xH₂O (0.085 g) was added to the reaction flask, followed by 2,3-butanedione (0.35 mL, 4.00 mmol). After these additions, the reaction was allowed to stir at 50° C. overnight. The next day the reaction was removed from heat and allowed to cool to room temperature. The reaction precipitate was collected via vacuum filtration and washed with cold MeOH and acetone until a white or pale-yellow solid remained on the filter paper. The precipitate was transferred to a vial and dried overnight under vacuum. After structural analysis, each molecule/monomer was confirmed to be the desired product. Analytical results are provided below for the various synthesized molecules.

2,5-bis(4-bromophenyl)-1,4-bis(4-n-decylphenyl)-1,4-dihydropyrrolo[3,2-b]pyrrole (Br₂DHPP): Analysis of the analytical data supported successful synthesis of the desired product and agrees with prior reports. Bell, K.-J. J., et al., Chem. Mater., 34: 8729-8739 (2022).

2,5-bis(4-cyanophenyl)-1,4-bis(4-n-decylphenyl)-1,4-dihydropyrrolo[3,2-b]pyrrole (DHPP 1): Yellow Solid. Yield: 1.88 g (63%)¹H NMR (400 MHz, CDCl₃), δ: 0.91 (t, 6H), 1.31-1.37 (m, 29H), 1.64-1.69 (m, 4H), 2.38 (s, 1H), 2.67 (t, 4H), 6.49 (s, 2H), 7.17-7.26 (m, 10H), 7.30 (d, 6H), 7.49 (d, J=8.5 Hz, 4H). The ¹H NMR spectrum agrees with prior reports.

### Example 7

Suzuki Cross-Coupling Procedure for π-extended DHPPs were synthesized using general Suzuki cross-coupling procedures.

FIG. 10 shows an example of a general Suzuki cross-coupling reaction for n-extended DHPP chromophores.

In a 10 mL round bottom flask equipped with a Teflon stir bar, DHPP (100 mg, 0.12 mmol), 2.2 molar equiv. of the corresponding aryl-boronic acid (0.264 mmol), 2 mol % of Pd(PPh₃)₂Cl₂, and one drop of Aliquat 336 were combined. A reflux condenser was attached along with a rubber septum, and the reaction flask was rendered inert via vacuum/refill cycles (3×) with Argon (Ar). Subsequently, 2 mL of degassed THE and 2M K₂CO₃(aq) were added via syringe, and the flask was placed in an oil bath set to 70° C. The reaction was allowed to stir for at least 20 h before being removed from the oil bath and cooled to r.t. The crude reaction mixture was added to a separatory funnel, and the flask was rinsed with DCM. The mixture was washed with H₂O (3×20 mL) and extracted with DCM. The organic layers were dried with Na₂SO₄, filtered, and concentrated via rotary evaporation. The product was then purified via column chromatography using a mixture of Hex:DCM as the eluent. Specific mobile phases are listed with the corresponding molecules.

The expected product was determined via TLC, and pure fractions were collected and concentrated via rotary evaporation. The solid product was then transferred to a vial and dried on the vacuum overnight. The expected products were then confirmed via NMR and elemental analysis, as provided below.

2,5-bis(4-phenyl)-2,5-bis(phenyl)-1,4-bis(4-n-decylphenyl)-1,4-dihydropyrrolo[3,2-b]pyrrole (2): Yellow Solid. Yield: 68.9 mg (79%) Mobile phase 3:1 (Hex:DCM). Melting point: 172.1-174.5° C. ¹H NMR (400 MHz, CDCl₃), δ: 0.91 (t, 8H), 1.30-1.36 (m, 35H), 1.66-1.69 (m, 5H), 2.66 (t, 4H), 6.49 (s, 2H), 7.22 (d, J=8.3 Hz, 4H), 7.28 (d, 6H), 7.34 (t, 6H), 7.44 (t, 4H), 7.50 (d, J=8.2 Hz, 4H), 7.62 (d, J=7.5 Hz, 4H). ¹³C NMR: (400 MHz, CDCl₃), δ: 14.1, 22.7, 29.5, 31.4, 31.9, 35.5, 94.6, 125.2, 126.75, 127.1, 128.3, 128.8, 129.1, 132.0, 132.8, 135.5, 137.7, 138.5, 140.5, 140.7. Anal. calc'd for C₆₂H₇₀N₂: C 88.31; H 8.37; N 3.32 Found: C 87.21; H 8.55; N 3.22.

2,5-bis(4-toyl)-2,5-bis(phenyl)-1,4-bis(4-n-decylphenyl)-1,4-dihydropyrrolo[3,2-b]pyrrole (3): Yellow Solid. Yield: 66.5 (64%) Mobile phase 3:1 (Hex:DCM). Melting point: 169.0° C. ¹H NMR (400 MHz, CDCl₃), δ: 0.91 (t, 7H), 1.30-1.36 (m, 32H), 1.65-1.69 (m, 4H), 2.41 (s, 6H), 2.66 (t, 4H), 6.47 (s, 2H), 7.19-7.31 (m, 22H), 7.46-7.52 (m, 9H). ¹³C NMR: (400 MHz, CDCl₃), δ: 14.1, 21.1, 22.7, 29.4, 29.5, 29.7, 31.4, 31.9, 35.5, 94.5, 125.2, 126.5, 126.6, 128.3, 129.0, 129.5, 131.9, 132.5, 135.6, 136.9, 137.8, 138.4, 140.5. Anal. calc'd for C₆₄H₇₄N₂: C 88.22; H 8.56; N 3.22 Found: C 88.06; H 8.65; H 3.23.

2,5-bis(4-methoxyphenyl)-2,5-bis(phenyl)-1,4-bis(4-n-decylphenyl)-1,4-dihydropyrrolo[3,2-b]pyrrole (4): Yellow Solid. Yield: 80.8 mg (75%) Mobile phase 2:1 (Hex:DCM). Melting point: 187.0-189.2° C. ¹H NMR (400 MHz, CDCl₃), δ: 0.90 (t, 7H), 1.30-1.36 (m, 33H), 1.65-1.69 (m, 5H), 2.65 (t, 4H), 3.87 (s, 7H), 6.46 (s, 2H), 6.98 (d, J=8.8 Hz, 4H), 7.20 (d, J=8.4 Hz, 4H), 7.28 (t, 15H), 7.44 (d, J=8.4 Hz, 4H), 7.54 (d, J=8.8 Hz, 4H). ¹³C NMR: (400 MHz, CDCl₃), δ: 14.1, 22.7, 29.4, 29.5, 29.7, 31.4, 31.9, 35.5, 55.4, 94.5, 114.2, 125.2, 126.2, 127.8, 128.3, 129.0, 131.9, 132.2, 133.3, 135.6, 137.8, 138.1, 140.5, 159.1. Anal. calc'd for C₆₄H₇₄N₂O₂: C 85.10; H 8.26; N 3.10 Found: C 85.19; H 8.33; N 3.17.

2,5-bis(4-trifluoromethylphenyl)-2,5-bis(phenyl)-1,4-bis(4-n-decylphenyl)-1,4-dihydropyrrolo[3,2-b]pyrrole (5): Yellow Solid. Yield: 69.5 mg (59%) Mobile phase 5:1 (Hex:DCM). Melting point: 190.0-192.0° C. ¹H NMR (400 MHz, CDCl₃), δ: 0.90 (t, 7H), 1.29-1.36 (m, 31H), 1.64-1.69 (m, 4H), 2.67 (t, 4H), 6.49 (s. 2H), 7.22 (d, J=8.5 Hz, 4H), 7.27 (d, 12H), 7.35 (d, J=8.4 Hz, 4H), 7.50 (d, J=8.4 Hz, 4H), 7.67-7.72 (m, 8H). ¹³C NMR: (400 MHz, CDCl₃), δ: 14.1, 22.7, 29.4, 29.5, 29.6, 31.4, 31.9, 35.5, 94.8, 125.2, 125.67-125.7, 126.9-127.0, 128.4, 129.1, 132.3, 133.7, 135.4, 136.9, 137.6, 140.8, 144.2. Anal. calc'd for C₆₄H₆₈F₆N₂: C 78.50; H 7.00; F 11.64; N 2.86 Found: C 78.07; H 7.15; N 2.85.

2,5-bis(4-cyanophenyl)-2,5-bis(phenyl)-1,4-bis(4-n-decylphenyl)-1,4-dihydropyrrolo[3,2-b]pyrrole (6): Vibrant yellow solid. Yield: 81.2 mg (76%) Mobile phase 1:1 (Hex:DCM). Melting point: 222.6-225.4° C. ¹H NMR (400 MHz, CDCl₃), δ: 0.90 (t, 7H), 1.29-1.40 (m, 33H), 1.64-1.69 (m, 5H), 2.66 (t, 4H), 6.50 (s, 2H), 7.21-7.28 (m, 19H), 7.35 (d, J=8.4 Hz, 5H), 7.49 (d, J=8.5 Hz, 4H), 7.69-7.73 (m, 9H). ¹³C NMR: (400 MHz, CDCl₃), δ: 14.1, 22.7, 29.4, 29.5, 29.6, 31.4, 31.9, 35.5, 94.9, 110.6, 119.0, 125.2, 126.9, 127.3, 128.4, 129.2, 132.5, 132.6, 134.1, 135.4, 136.3, 137.5, 140.9, 145.1. Anal. calc'd for C₆₄H₆₈N₄: C 86.05; H 7.67; N 6.27 Found: C 85.79; H 7.86; N 6.09.

2,5-bis(4-napthelene)-2,5-bis(phenyl)-1,4-bis(4-n-decylphenyl)-1,4-dihydropyrrolo[3,2-b]pyrrole (7): Yellow Solid. Yield: 94 mg (83%) Mobile phase 4:1 (Hex:DCM). Melting point: 138.2-140.7° C. ¹H NMR (400 MHz, CDCl₃), δ: 0.89 (t, 6H), 1.27-1.36 (m, 31H), 1.67-1.70 (m, 4H), 2.68 (t, 4H), 6.55 (s, 2H), 7.25 (d, J=8.3 Hz, 4H), 7.34 (d, J=8.4 Hz, 4H), 7.39 (s, 8H), 7.45-7.56 (m, 9H), 7.87 (d, J=8.1 Hz, 2H), 7.92 (d, J=7.4 Hz, 2H), 7.99 (d, J=8.3 Hz, 2H). ¹³C NMR: (400 MHz, CDCl₃), δ: 14.1, 22.7, 29.3-29.4, 29.5, 29.6, 31.4, 31.9, 35.5, 94.8, 125.2, 125.4, 125.7, 125.97-126.04, 126.9, 127.6, 128.0, 128.3, 129.1, 129.9, 131.6, 132.0, 132.8, 133.9, 135.6, 137.8, 138.3, 140.0, 140.6. Anal. calc'd for C₇₀H₇₄N₂: C 89.12; H 7.91; N 2.97 Found: C 84.85; H 7.86; N 2.83.

2,5-bis(4-thiophene)-2,5-bis(phenyl)-1,4-bis(4-n-decylphenyl)-1,4-dihydropyrrolo[3,2-b]pyrrole (8): Yellow Solid. Yield: 73.6 mg (72%) Mobile phase 4:1 (Hex:DCM). Melting point: 187.8-189.4° C. ¹H NMR (400 MHz, CDCl₃), δ: 0.91 (t, 7H), 1.30-1.36 (m, 31H), 1.64-1.69 (m, 4H), 2.66 (t, 4H), 6.45 (s, 2H), 7.07-7.09 (m, 2H), 7.20-7.30 (m, 21H), 7.49 (d, J=8.4 Hz, 4H). ¹³C NMR: (400 MHz, CDCl₃), δ: 14.1, 22.7, 29.4, 29.5, 29.7, 31.4, 31.9, 35.5, 94.5, 122.7, 125.2, 125.5, 128.0, 128.3, 129.1, 131.9, 132.1, 132.9, 135.5, 137.7, 140.6. Anal. calc'd for C₅₈H₆₆N₂S₂: C 81.45; H 7.78; N 3.28; S 7.50 Found: C 81.56; H 7.86; N 3.21; S 7.69.

2,5-bis(3,5-fluorophenyl)-2,5-bis(phenyl)-1,4-bis(4-n-decylphenyl)-1,4-dihydropyrrolo[3,2-b]pyrrole (9): Yellow Solid. Yield: 89.3 mg (83%) Mobile phase 5:1 (Hex:DCM). Melting point: 156.9-159.4° C. ¹H NMR (400 MHz, CDCl₃), δ: 0.87 (t, 7H), 1.27-1.33 (m, 31H), 1.61-1.67 (m, 4H), 2.64 (t, 4H), 6.46 (s, 2H), 6.72-6.77 (m, 2H), 7.06-7.11 (m, 4H), 7.18-7.25 (m, 10H), 7.29 (d, J=8.4 Hz, 4H), 7.41 (d, J=8.4 Hz, 4H). ¹³C NMR: (400 MHz, CDCl₃), δ: 14.1, 22.7, 29.3-29.4, 29.5, 29.6, 31.4, 31.9, 35.5, 94.8, 102.2 (d, J=25.5 Hz), 109.4 (t, J=7.0 Hz), 109.6 (d, J=7.0 Hz), 125.2, 126.6, 128.4, 129.2, 132.4, 133.9, 135.4, 136.1, 137.6, 140.8, 144.0, 162.1 (d, J=13.3 Hz), 164.6 (d, J=13.1 Hz), 164.7-162.2 (d, J=248.5 Hz), 164.5-162.1 (d, J=249.5 Hz). Anal. calc'd for C₆₂H₆₆F₄N₂: C 81.37; H 7.27; N 3.06 Found: C 81.07; H 7.34; N 3.05.

2,5-bis(3-trifluoromethylphenyl)-2,5-bis(phenyl)-1,4-bis(4-n-decylphenyl)-1,4-dihydropyrrolo[3,2-b]pyrrole (10): Yellow Solid. Yield: 101.1 mg (88%) Mobile phase 5:1 (Hex:DCM). Melting point: 135.2-141.1° C. ¹H NMR: (400 MHz, CDCl₃), δ: 0.91 (t, 7H), 1.30-1.37 (m, 31H), 1.65-1.72 (m 4H), 2.67 (t, 4H), 6.50 (s, 2H), 7.22-7.29 (m, 9H), 7.35 (d, J=8.4 Hz, 4H), 7.50 (d, J=8.4 Hz, 4H), 7.53-7.61 (m, 4H), 7.78 (d, J=7.5 Hz, 2H), 7.85 (s, 2H). ¹³C NMR: (400 MHz, CDCl₃), δ: 14.1, 22.7, 29.35-29.39, 29.5, 29.6, 31.4, 31.9, 35.5, 94.8, 122.9, 123.56-123.60, 123.7, 125.2, 125.6, 126.8, 128.4, 129.1-129.2, 130.0, 131.0, 131.3, 132.3, 133.6, 135.4, 136.9, 137.6, 140.8, 141.5. Anal. calc'd for C₆₄H₆₈F₆N₂: C 78.50; H 7.00; N 2.86 Found: C 78.24; H 7.14; N 2.85.

2,5-bis(3-methoxyphenyl)-2,5-bis(phenyl)-1,4-bis(4-n-decylphenyl)-1,4-dihydropyrrolo[3,2-b]pyrrole (11): Yellow Solid. Yield: 77.2 mg (75%) Mobile phase 2:1 (Hex:DCM). Melting point: 121.7-123.8° C. ¹H NMR (400 MHz, CDCl₃), δ: 0.89-0.92 (m, 9H), 1.30-1.36 (m, 37H), 1.66-1.69 (m, 5H), 2.66 (t, 4H), 3.88-3.90 (m, 7H), 6.48 (s, 2H), 6.88-6.91 (m, 2H), 7.15 (t, 2H), 7.19-7.22 (m, 7H), 7.26-7.38 (m, 13H), 7.48-7.50 (m, 4H). ¹³C NMR: (400 MHz, CDCl₃), δ: 11.2, 14.1, 22.7, 29.35-29.38, 29.5, 29.6, 31.4, 31.9, 35.5, 55.3, 94.6, 112.6, 119.4, 125.2, 126.8, 128.3, 129.1, 129.7, 132.1, 133.0, 135.5, 137.7, 138.3, 140.6, 142.3, 159.9. Anal. calc'd for C₆₄H₇₄N₂O₂: C 85.10; H 8.26; N 3.10 Found: C 84.23; H 8.30; N 3.06.

2,5-bis(2-methoxyphenyl)-2,5-bis(phenyl)-1,4-bis(4-n-decylphenyl)-1,4-dihydropyrrolo[3,2-b]pyrrole (12): Yellow Solid. Yield: 90.1 mg (87%) Mobile phase 2:1 (Hex:DCM). Melting point: 118.7-120.9° C. ¹H NMR (400 MHz, CDCl₃), δ: 0.91 (t, 7H), 1.30-1.36 (m, 31H), 1.66-1.69 (m, 4H), 2.66 (t, 4H), 3.84 (s, 6H), 6.47, (s, 2H), 6.99-7.06 (m, 4H), 7.21 (d, J=8.4 Hz, 4H), 7.28-7.32 (m, 11H), 7.34-7.37 (m, 3H), 7.45 (d, J=8.4 Hz, 4H). ¹³C NMR: (400 MHz, CDCl₃), δ: 11.2, 14.1, 22.7, 29.35-29.38, 29.5, 29.6, 31.4, 31.9, 35.5, 55.3, 94.6, 112.5-112.6, 119.4, 125.2, 126.8, 128.3, 129.1, 129.7, 132.1, 133.0, 135.5, 137.7, 138.3, 140.6, 142.3, 159.9. Anal. calc'd for C₆₄H₇₄N₂O₂: C 85.10; H 8.26; N 3.10 Found: C 85.04; H 8.41; N 3.11.

The yields for the para-substituted chromophores were about 60% up to about 76%, which was consistent with previous reports. DHPP 7 and 8 were successfully synthesized with yields of about 83% and about 72%, respectively. The naphthalene yield was high as consistent with previous naphthalene derivatives, demonstrating that there was not significant steric constraints for this synthesis. As a way to investigate the influence of the π-extension on the structure-property relationships of DHPPs, DHPP 1 also was synthesized to compare with DHPP 6.

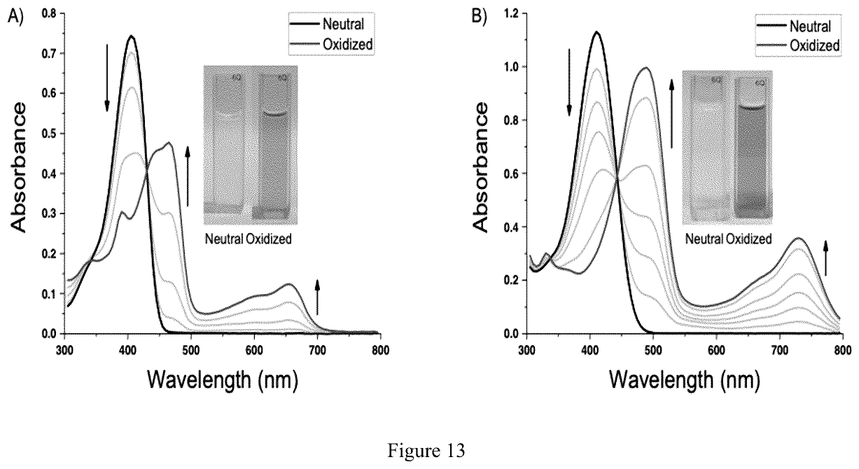

Purification of each DHPP chromophore was accomplished via column chromatography, and structure and purity were confirmed with ¹H and ¹³C NMR. FIG. 11 is the NMR spectra for DHPP 1 where the diagnostic pyrrolopyrrole peak at of about 6.5 ppm is present and all other protons match previous reports. FIG. 11 shows the NMR spectra for the starting material Br₂DHPP, and all the protons align with previous reports, as well. Bell, K.-J. J., et al., Chem. Mater., 34: 8729-8739 (2022). The ¹H and ¹³C NMR spectra for the π-extended DHPPs are shown in FIGS. 13-34. All expected protons and carbons were accounted for in the NMR spectra. The diagnostic DHPP peak at of about 6.5 ppm was retained for the π-extended DHPPs, which indicated there was no observation of side product yield as a result of the pyrrolopyrrole protons being active in direct arylation reactions.

Purity was determined using elemental analysis (EA). Theoretical and calculated values were found to be statistically similar and validated the purity of the molecules. Melting points were used as another analytical technique for determining purity. All molecules had narrow melting point ranges attributed to a high level of purity. (Table 1 above). Each of the synthesized DHPPs had melting points of about 130° C. up to about 220° C. similar to other DHPPs.

Variations in the electronic character and the positioning of the substituents altered the strength and stability of the intermolecular forces that influenced the melting point. As a non-limiting example, molecules substituted ortho or meta to the DHPP core exhibited melting points lower than those substituted at the para position. Additionally, chromophores substituted with electron-withdrawing substituents displayed higher melting temperatures.

The three analytical techniques confirmed that the molecules were the desired products and were present at a high level of purity as desired for aims of this disclosure.

Table 1 above shows melting point ranges for DHPPs 2-12. The narrow temperature ranges were consistent with a high level of purity.

### Example 8

When studying the optical properties of DHPPs 7-12, changing the substitution position was found not to alter the optical properties significantly. For example, and as shown in FIG. 11 and Table 2 (above), the methoxy-functionalized DHPPs, DHPP 4 and DHPP 11, had similar λₘₐₓ values of about 382 nm up to about 383 nm while DHPP 12, with a methoxy group at the meta-position, had a λₘₐₓ of about 378 nm. The blue-shift from DHPP 11 to DHPP 12 was attributed to increased torsional angles that disrupted the chromophores conjugation by positioning the OMe at the ortho-position with respect to the DHPP core. The same trend was seen with the CF₃-functionalized coupling partners, with the para-functionalized chromophore DHPP 5 having a λₘₐₓ of about 397 nm while the meta-functionalized chromophore DHPP 10 had a λₘₐₓ of about 391 nm. The 6 nm difference between the para- and meta-CF₃ chromophores was likely due to a decreased push-pull effect by removing the electron-withdrawing substituent from the conjugation pathway.

A multi-fluorinated coupling partner, DHPP 9, yielded a slightly red-shifted L. of about 394 nm compared to DHPP 10 (λₘₐₓ of about 391 nm) due to the increased electronegativity of the fluorine atoms that increases the push-pull effect.

The naphthalene- and thienyl-functionalized DHPPs, DHPP 7 and DHPP 8 respectively, display λₘₐₓ values of about 379 nm and of about 400 nm (FIG. 11B and Table 2). DHPP 7 showed a slightly red-shifted absorbance compared to other PAH-functionalized DHPPs (λₘₐₓ of about 369 nm) due to extending the π-conjugation while the increased quinoidal character of thiophene, and the subsequent reduction in dihedral angles, resulted in the red shift observed for DHPP 8 compared to other Tc-extended DHPPs with electron-donating characteristics.

The synthesis of various DHPPs displayed the robust nature of functionalizing DHPPs through Pd-catalyzed cross-coupling reactions with diverse aromatic functionalities. The structural diversity influenced the optical properties of the neutral molecules.

FIG. 11 shows UV-vis absorbance spectra for the varying substituent choice and position of DHPP chromophores.

### Example 9

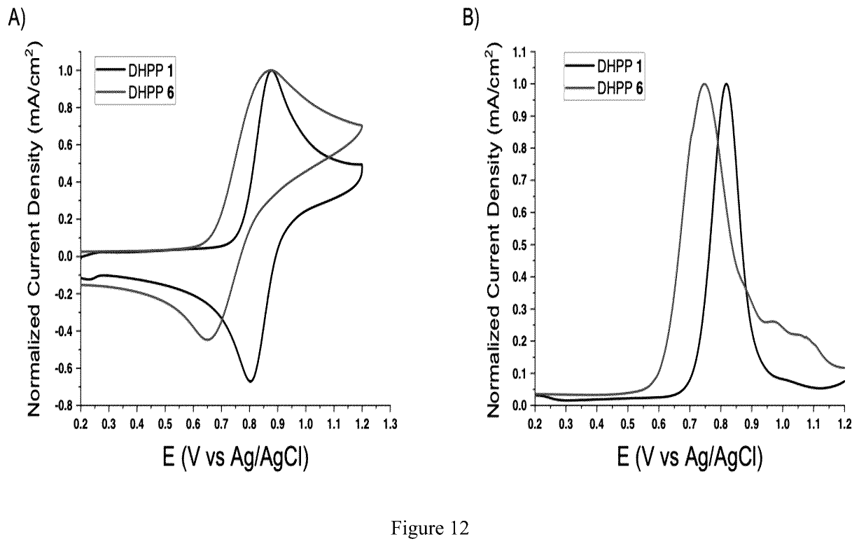

Electrochemistry analyses were performed. DHPP 1 and DHPP 6 were compared to understand the influence of increased conjugation on the redox activity. As shown in FIG. 12A, DHPP 1 had an onset of oxidation of about 0.68 V which was slightly higher compared to DHPP 6 whose onset of oxidation was measured to be of about 0.58 V (vs. Ag/AgCl) (Table 3 above). The lower onset of oxidation from DHPP 1 to DHPP 6 resulted from the increased π-conjugation which reduced the voltage required to remove an electron. By increasing the π-conjugation, the redox properties of DHPPs were manipulated. This shows that this approach allowed controlling the redox properties of DHPP molecules.

Table 3 above shows electronic properties of DHPP 1 and DHPP 6 obtained from electrochemical and optical characterizations showing the influence of 7-conjugation on redox activity.

FIG. 12 shows a comparison of redox response of DHPP 1 and DHPP 6 using (A) CV and (B) DPV in a 0.5 M TBAPF₆/DCM supporting electrolyte against Fc/Fc⁺ calibration illustrating the effect of increasing the π-conjugation on the redox activity of DHPPs.

### Example 10

Solution oxidation studies were performed. A comparison of how the increased conjugation length influenced radical cation properties was addressed first by comparing changes in the absorbance of DHPP 1 and DHPP 6. FIG. 13 displays the transition of the neutral UV-vis absorbance spectra to the absorbance spectra of the molecules as they were oxidized. Upon oxidization, DHPP 1 and DHPP 6 shifted further into the visible region of the EMS and displayed characteristics of SOMO-α→LUMO-α and SOMO-β→LUMO-β transitions.

For DHPP 1, the SOMO-α→LUMO-α at about 465 nm was broad and less prominent than the same transition associated with DHPP 6 (about 490 nm) which may be attributed to the radical cation of DHPP 1 having a smaller molar absorptivity compared to the radical cation of DHPP 6. The red shift in the maximum absorbance for the SOMO-α→LUMO-α (λₘₐₓ^{α}) from DHPP 1 to DHPP 6 was attributed to the increase in the conjugation length (Table 4 above). Going from the neutral absorbance to the SOMO-α→LUMO-α, DHPP 6 had a more exacerbated red shift with an about 80 nm shift while DHPP 1 only had an about 60 nm shift.

The SOMO-β transitions followed the same trend as the SOMO-α transitions where absorbance maximum for the SOMO-β→LUMO-β (λₘₐₓ^{β}) of DHPP 1 was located at about 655 nm and the (λₘₐₓ^{β}) of DHPP 6 at about 730 nm. There was a difference in the absorbance profile of the SOMO-β→LUMO-β with DHPP 1 having a lower intensity at about 0.2 absorbance units while DHPP 6 had a more intense SOMO-β transition. The more intense absorbance for DHPP 6 was attributed to the difference in molar absorptivity and the increased rotational freedom of the π-extended system, which enabled more allowed excited state geometries.

Due to the differences in the λₘₐₓ and absorbance profiles of the SOMO→LUMO transitions, it was determined that manipulation of the π-conjugation of DHPP chromophores provided control of the absorbance profiles.

Table 4 above shows UV-vis and color coordinate data for DHPP 1 and DHPP 6. The neutral and oxidized λₘₐₓ values correspond to the SOMO-α→LUMO-α and SOMO-β→LUMO-β, while the neutral and oxidized color coordinates were calculated based on mid-day lighting standards (D50 illuminant as a 2° observer).

FIG. 13 shows solution oxidation spectra of (A) DHPP 1 and (B) DHPP 6 in DCM using 0.06 mg/mL Fe(ClO₄)₃·xH₂O in ethyl acetate as the dopant. These spectra displayed changes in the UV-vis absorbance spectra with increasing dopant concentration to elucidate and compare the ability of DHPPs to be chemically doped.

### Example 11

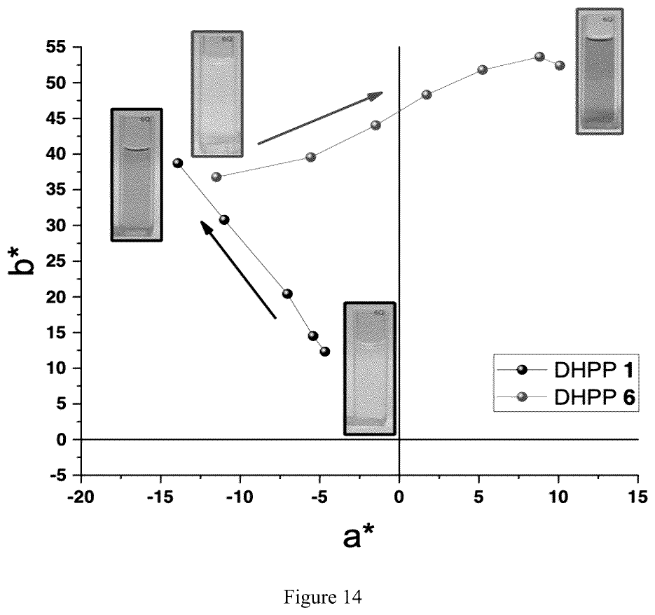

For DHPP 1, the neutral solution was found to have color coordinates of 100, −5, 12 (Table 4 above). Within the coordinate diagram, these values corresponded to a decreased saturation level, and the solution being perceived as nearly colorless or transmissive (FIG. 14). The π-extended DHPP 6 experiences color coordinates of 100, −11, 37, which was a light-yellow color when placed on the coordinate diagram and was consistent with the red-shifted UV-vis data reported in FIG. 5. This coloration was confirmed by the photographs within the insets of FIG. 14. Upon oxidation, both DHPPs displayed a shift in their solution color, resulting in L*a*b* color coordinates of 93, −14, 39 for DHPP 1 and 83, 10, 52 for DHPP 6. The color transitions for these two DHPPs were transmissive-to-yellow-green for DHPP 1, while DHPP 6 exhibited a yellow-to-golden-yellow color change. The green color was from the dual-band absorbance within the visible region of the EMS with a transmissive window from about 500 nm up to about 550 nm as measured via UV-vis for the oxidized DHPP 1. The red shift from DHPP 1 to DHPP 6 illustrated in the UV-vis absorbance spectra was further shown where the location of the colors of the oxidized chromophores were in two separate quadrants. The results showed the differences in the color profile for the two DHPPs and the ability to fine-tune the radical cation absorbance through the conjugation length. This showed how coupling partner selection impacted the radical cation of DHPPs and ultimately color of oxidized solutions.

FIG. 14 shows color coordinate data for DHPP 1 and DHPP 6 as a function of dopant concentration that quantified the color difference experienced by the π-extended systems.

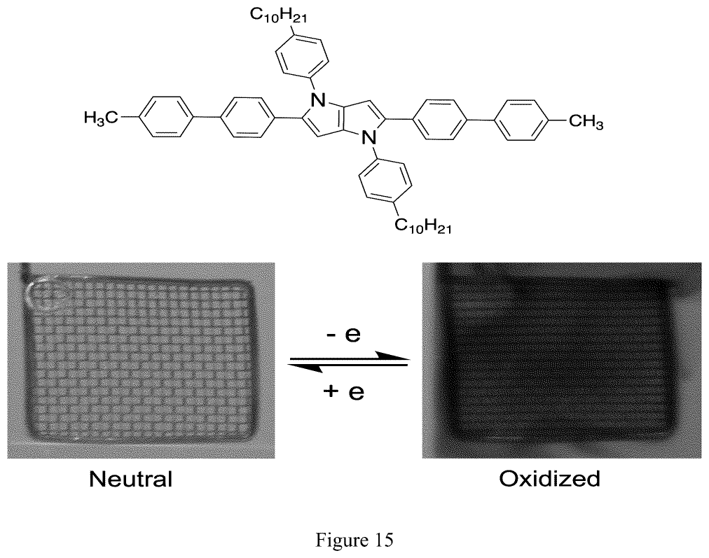

FIG. 15 shows electrochemical switching experiments using an OTTLE of the color-controlled, high-contrast DHPP chromophore DHPP 3.

### Example 12

Substituents that were electron-neutral, electron-donating, or a combination of electron-donating and -withdrawing were incorporated to understand how peripheral substituents of DHPP molecules dictated the position and shape of the resulting radical cation. Influencing optoelectronic properties of DHPPs through coupling partners was explored in Hawks et al. Other studies studied ACE molecules and electrochromic polymers. Christiansen, D. T., et al., J. Am. Chem. Soc., 141: 3859-3862 (2019); Nhon, L., et al., J. Chem. Phys., 154: 054110 (2021); Beaujuge, P. M., et al., Chem. Rev., 110(1): 268-320 (2010).

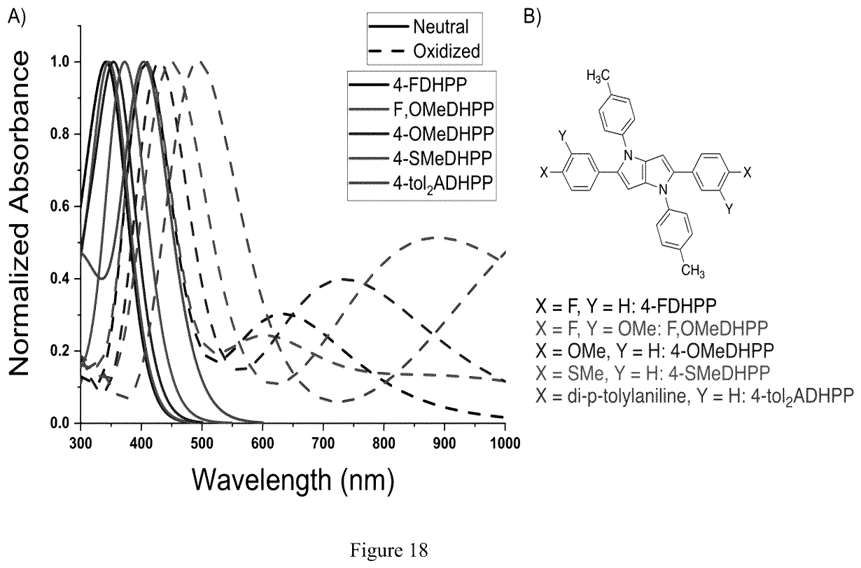

Five DHPPs with varying functionalities were synthesized according to the present disclosure. TDDFT was used to support synthesis, and results from the calculations are presented in FIG. 18. Excluding 4-tol₂ADHPP, which was predicted to absorb within the visible region of the electromagnetic spectrum (EMS), the remaining molecules screened in these calculations were expected to absorb in the UV region, demonstrating the first requirement for anodically coloring materials (FIG. 18). Upon oxidation, the SOMO-α→LUMO-α shifted from the UV region to or beyond about 400 nm with varying intensities and positions of the SOMO-β→LUMO-β absorbances. The variation in the SOMO-β transition signified a difference in the color profile of the molecules and supported initial hypotheses. FIG. 18 shows the predicted dependence of the SOMO-β→LUMO-β absorbance on the choice of functionality that led to color control of DHPP electrochromes. When comparing 4-FDHPP to 4-OMeDHPP, 4-SMeDHPP and 4-tol₂ADHPP, the SOMO-β of the electron-donating substituents were red-shifted into the NIR, which supported that changes to functionality enabled color control. These calculations were compared to syntheses and evaluations of structure-property relationships of DHPP electrochromes.

FIG. 18 shows (FIG. 18A) theoretical neutral and oxidized absorbance spectra for the DHPPs screened for potential color control using B3LYP-631G* and (FIG. 18B) general structures used for the calculations of the DHPPs chromophores reported in (FIG. 18A). The R groups were reduced to CH₃ groups for simplifying structural input into TDDFT calculations. The neutral spectra are shown as the solid lines while the oxidized are represented as dashed lines.

### Example 13

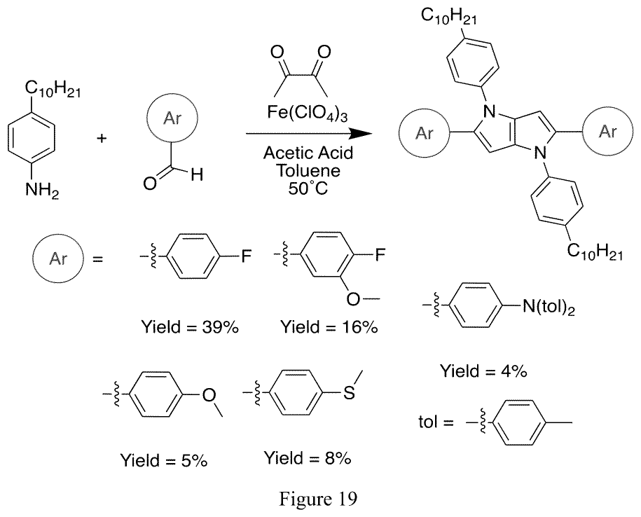

Motivated by the theoretical results showing UV-vis absorbance transitions of the neutral state falling within the UV region of the EMS, the five DHPP molecules were synthesized using the reaction procedure adopted by Bell et al. and Tasior (FIG. 19). Bell, K.-J. J., et al., Chem. Mater., 34: 8729-8739 (2022); Tasior, M., et al., J. Org. Chem., 85: 13529-13543 (2020). To demonstrate the robust synthesis and ability to alter the functionality of DHPPs using various aldehydes, a whole family of DHPPs was synthesized whose results and discussion can be found in the FIGS. 50 and 52-61 and Tables 7-10 below.

TABLE 7
The onsets of oxidation, calculated energy levels, and energy gaps
for fluorinated DHPP molecules. This demonstrates that varying
the electron-withdrawing nature of the DHPP periphery alters the
redox properties without sacrificing desired optical gaps.

| | | HOMO | LUMO | E_{gap} |
|---|---|---|---|---|
| Chromophore | Eₒₙₛₑₜ^{ox} (V) | (eV)^{a} | (eV)^{b} | (eV)^{c} |
| 4-FDHPP | 0.42 | −5.5 | −2.4 | 3.1 |
| 3,4-F₂DHPP | 0.51 | −5.6 | −2.5 | 3.1 |
| 3,4,5-F₃DHPP | 0.70 | −5.8 | −2.7 | 3.1 |

^{a}Calculated given HOMO = −(Eₒₙₛₑₜ^{ox} + 5.12 eV);
^{b}Calculated from absorbance onset given eV = 1240//λₒₙₛₑₜ + HOMO;
^{c}Calculated from (LUMO − HOMO); all equations are adopted from Cardona and coworkers. Cardona, C. M., et al., Adv. Mater., 23(20): 2367-2371 (2011).

TABLE 8
Optical data for the fluorinated DHPPs, including the absorbance
λₘₐₓ and color coordinates for the neutral and oxidized species.

| | | | | Color Coordinates | |
|---|---|---|---|---|---|
| | λₙₑᵤ^{max} | λₒₓ^{max} (nm) | | Neu. | Ox. |
| Chromophore | (nm) | SOMO-α | SOMO-β | (L*a*b*) | (L*a*b*) |
| 4-FDHPP | 345 | 450 | 650 | 100, | 91, |
| | | | | 0, 0 | −27, 38 |
| 3,4-F₂DHPP | 350 | 450 | 655 | 100, | 91, |
| | | | | 0, 0 | −26, 40 |
| 3,4,5-F₃DHPP | 355 | 445 | 650 | 100, | 96, |
| | | | | 0, 0 | −12, 19 |

TABLE 9
Electronic properties for DHPPs with varying substituents.

| | Eₒₙₛₑₜ^{ox} | HOMO | LUMO | E_{gap} |
|---|---|---|---|---|
| Chromophore | (V) | (eV)^{a} | (eV)^{b} | (eV)^{c} |
| 4-MeDHPP | 0.41 | −5.5 | −2.4 | 3.1 |
| 4-CF₃DHPP | 0.67 | −5.8 | −2.9 | 2.9 |
| F₅DHPP | 0.93 | −6.1 | −2.9 | 3.2 |
| EstDHPP | 0.62 | −5.7 | −3.0 | 2.7 |

^{a}Calculated given HOMO = −(Eₒₙₛₑₜ^{ox} + 5.12 eV);
^{b}Calculated from absorbance onset given eV = 1240/λₒₙₛₑₜ + HOMO;
^{c}Calculated from (LUMO − HOMO); all equations are adopted from Cardona and coworkers. Cardona, C. M., et al., Adv. Mater., 23(20): 2367-2371 (2011).

TABLE 10
Optical data for all extra ACE molecules, including the
λₘₐₓ and color coordinates for the neutral and oxidized species.

| | λₙₑᵤ^{max} | | | | |
|---|---|---|---|---|---|
| Chromophore | (nm) | λₒₓ^{max} (nm) | | Color Coordinates | |
| 4-MeDHPP | 350 | 465 | 680 | 100, | 90, |
| | | | | −0, 0 | −22, 58 |
| 4-CF₃DHPP | 375 | 445 | 629 | 100, | 86, |
| | | | | −0, 1 | −23, 41 |
| F₅DHPP | 330 | N/A | N/A | 100, | N/A |
| | | | | 0, 1 | |
| AcDHPP | 355 | 460 | 670 | 100, | 85, |
| | | | | 0, 0 | −36, 70 |
| EstDHPP | 400 | 465 | 660 | 100, | 89, |
| | | | | −6, 16 | −22, 60 |

All DHPPs were synthesized, but lower yields were obtained for the electron-rich functionalized DHPPs which was consistent with literature. The low yields were attributed to the increased stability of the Schiff base intermediate due to electron-donation into the system by the electron-donating substituent that hindered the reactivity. An adequate amount of material to proceed with characterization was produced.

Purity and connectivity of all five DHPPs were confirmed with NMR (FIGS. 63-72). The diagnostic DHPP peak of about 6.5 ppm was present in all of the ¹H NMR spectra, indicative of the two protons on the pyrrolopyrrole ring. The ¹³C NMR spectra of the DHPPs with fluorine substituents were analyzed for C—F coupling and the results were reported in Tables 11 and 12 below.

TABLE 11
J-coupling constants and peak assignments for the
¹³C NMR of 4-FDHPP. Calculated coupling constants
are consistent with aromatic carbon-fluorine coupling constants
reported by Weigert and coworkers. Weigert, F. J., et al.,
J. Am. Chem. Soc., 93(10): 2361-2369 (1971).

| Chemical | | Coupling | |
|---|---|---|---|
| Shift (ppm) | Peak | Constant J (Hz) | Interaction |
| 161.5 | d | 245.9 | ¹J (C—F) |
| 115.1 | d | 21.5 | ²J (C—C—F) |
| 129.8 | d | 7.8 | ³J (C—C—C—F) |
| 130.0 | d | 3.3 | ⁴J (C—C—C—C—F) |

TABLE 12
J-coupling constants and peak assignments for the
¹³C NMR of F, OMeDHPP. Calculated coupling constants
are consistent with aromatic carbon-fluorine coupling constants
reported by Weigert and coworkers. Weigert, F. J., et al.,
J. Am. Chem. Soc., 93(10): 2361-2369 (1971).

| Chemical | | Coupling | |
|---|---|---|---|
| Shift (ppm) | Peak | Constant J (Hz) | Interaction |
| 151.1 | d | 246 | ¹J (C—F) |
| 147.0 | d | 10.9 | ²J (C—C—F) |
| 115.8 | d | 18.5 | ²J (C—C—F) |
| 120.4 | d | 6.5 | ³J (C—C—C—F) |
| 130.2 | d | 3.8 | ⁴J (C—C—C—C—F) |

Analysis of the C—F coupling constants were consistent with previous reports. Elemental analysis was obtained for DHPPs with significant structural differences from previously reported DHPPs, and the theoretical values matched the experimental with a high level of accuracy. The ¹H NMR spectra for 4-OMeDHPP and 4-MeDHPP were consistent with previously synthesized DHPPs except for the alkyl region due to the increased length of the alkyl chain used in the present disclosure.

FIG. 19 shows a reaction scheme for the synthesis of DHPP molecules with varying functional groups using the optimized multicomponent reaction conditions.

### Example 14

Electrochemical studies were performed to understand redox properties. Electrochemistry measurements were done using cyclic voltammetry (CV) and differential pulse voltammetry (DPV) to determine the redox activity of the family of DHPPs. DHPPs are an abundantly electron-rich conjugated system. Additionally, electrochemical measurements were used to study the redox activity of DHPPs.

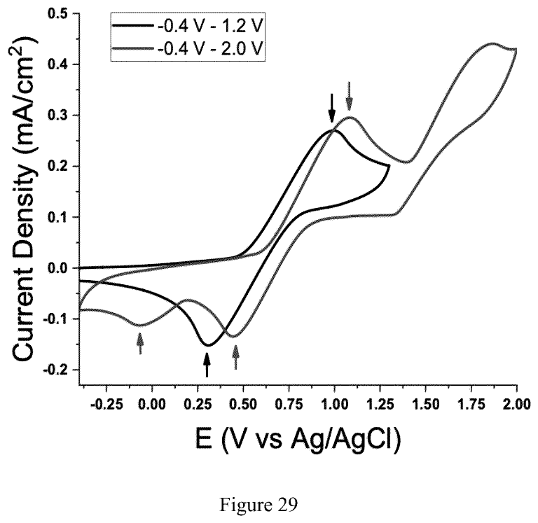

First, CV was measured for 4-FDHPP from −0.4 V to 1.2 V (black trace) to investigate the “reversibility” of the molecule (FIG. 29) that would be important for repeated electrochemical cycling in electrochromic measurements. Previous electrochemical investigations show some DHPPs, as well as other molecular chromophores, display dication formation that leads to electrochemical degradation of the chromophores. Dication behavior is investigated herein in addition to the radical cation to provide information on the operational range of electrochemical measurements and in device implementation.

The electrochemical window was expanded for an experimental window from −0.4 V to 2 V vs. Ag/AgCl. The expanded electrochemical window led to the formation of two new oxidation peaks and a shift in the onset of oxidation from about 0.5 V to about 0.55 V in addition to the formation of two new reduction peaks at about 0.4 V and about −0.1 V. The results were consistent with previous reports and supported the formation of new species or the degradation of our molecule.

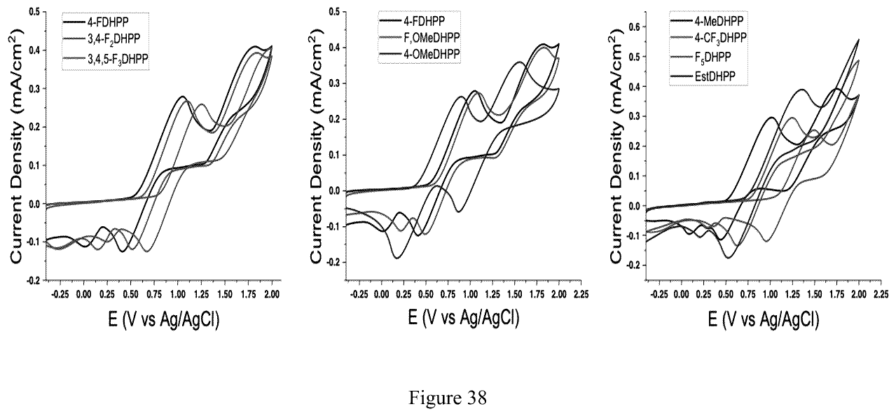

The CV data collected using the expanded experimental window for the rest of the molecules are shown in FIG. 38. and each molecule displays similar trends. These results support needing careful selection of experimental parameters.

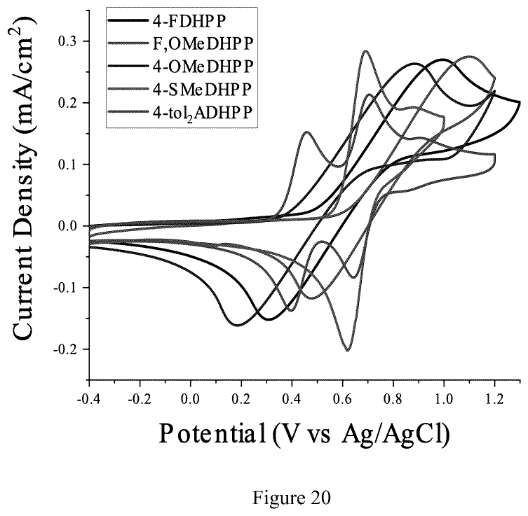

The ability to manipulate the onset of oxidation of a DHPP with the variation between electron-withdrawing and -donating groups is shown in FIG. 20 and Table 13 below. 4-OMeDHPP exhibited the lowest Eₒₙₛₑₜ^{ox} at about 0.26 V (vs. Ag/AgCl) due to the strong electron-donating nature of the methoxy group that enabled efficient removal of an electron at a lower potential (Table 13). The variations in the redox activity support controlling the redox properties with structural modifications.

FIG. 20 shows the (A) CV and (B) DPV of 4-FDHPP, F,OMeDHPP, 4-OMeDHPP, 4-SMeDHPP and 4-tol₂ADHPP using an Ag/AgCl reference electrode and a 0.5 M TBAPF₆/DCM supporting electrolyte. This figure demonstrates the ability to manipulate the redox properties of DHPPs depending on the functionalization at the para-position.

TABLE 13
Electronic properties for DHPPs with varying substituents.

| | Eₒₙₛₑₜ^{ox} | HOMO | LUMO | E_{gap} |
|---|---|---|---|---|
| Chromophore | (V) | (eV)^{a} | (eV)^{b} | (eV)^{c} |
| 4-FDHPP | 0.42 | −5.5 | −2.4 | 3.1 |
| F,OMeDHPP | 0.49 | −5.6 | −2.6 | 3.0 |
| 4-OMeDHPP | 0.26 | −5.4 | −2.3 | 3.1 |
| 4-SMeDHPP | 0.51 | −5.6 | −2.7 | 2.9 |
| 4-tol₂ADHPP | 0.28 | −5.4 | −2.6 | 2.8 |

^{a}Calculated given HOMO = −(Eₒₙₛₑₜ^{ox} + 5.12 eV);
^{b}Calculated from absorbance onset given eV = 1240/λₒₙₛₑₜ + HOMO;
^{c}Calculated from (LUMO − HOMO); all equations are adopted from Cardona and coworkers. Cardona, C. M., et al., Adv. Mater., 23(20): 2367-2371 (2011).

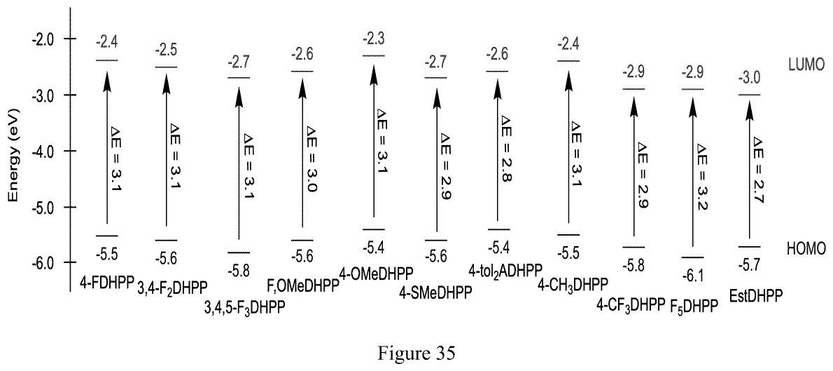

While the onset of oxidation shifted depending on the electronic character, the energy band gaps remained consistent, as displayed in FIG. 35 and tabulated in Table 13.

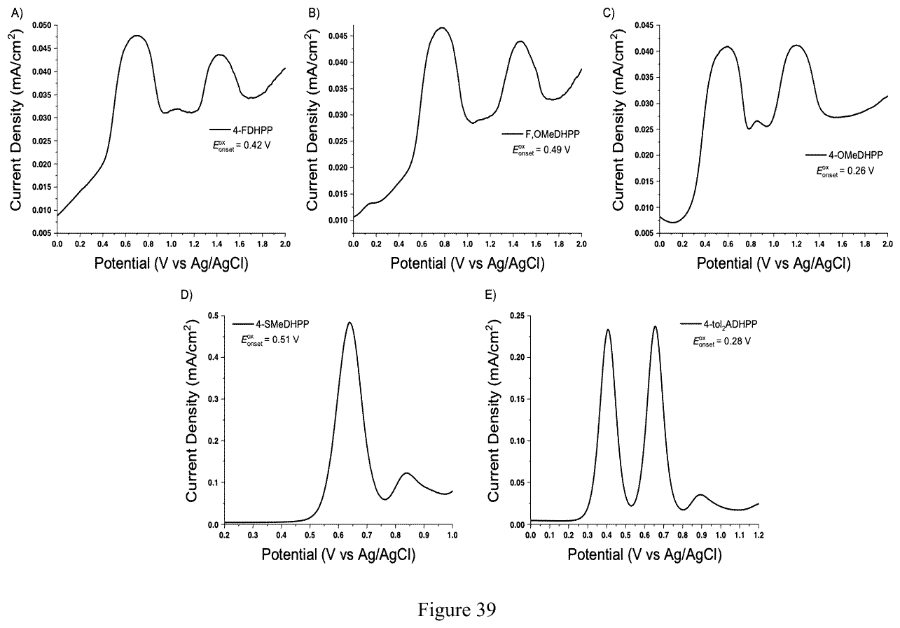

FIG. 35 uses the calculated HOMO, LUMO, and energy gap data to illustrate how substituents influence the electronic character of this family of DHPPs. DPV is notorious for having an increased level of precision when determining the onsets of oxidation and therefore giving a more pronounced Eₒₙₛₑₜ^{ox}, as displayed in FIG. 39. These onset values were used in the HOMO/LUMO and band gap calculations.

The DHPPs had E_{gap} values on the range of about 2.7 to about 3.1 eV, with the a difference coming from the slight narrowing of the E_{gap} for EstDHPP ΔE=2.7. The narrowing of the HOMO/LUMO gap with electron-withdrawing substituents was from the push-pull effect between the electron-rich pyrrolopyrrole backbone and the electron-deficient substituents. Compared to other molecules, these DHPP molecules had a relatively wide E_{gap} which corresponded to absorbance within the UV region of the EMS. The energy band gaps remained consistent regardless of the functionality substituents on the benzene rings attached at the 2,5-positions of the DHPP. This showed that the structural changes enabled manipulation of other optoelectronic properties without sacrificing the required ACE property of absorbing in the UV portion of the EMS.

### Example 15

Calculated and experimental optical properties were compared. A comparison of the calculated and the experimental UV-vis absorbance spectra was performed. Each of the calculated neutral spectra aligned with experimental measurements (FIG. 36). 4-OMeDHPP had a (λₘₐₓ^{neu}) of about 345 nm which was the same as 4-FDHPP, while F,OMeDHPP had a slightly red-shifted (λₘₐₓ^{neu}) of about 350 nm (FIG. 36 and Table 14 below).

TABLE 14
Optical data for all ACE molecules, including the λₘₐₓ
and color coordinates for the neutral and oxidized species.

| | | | | Color Coordinates | |
|---|---|---|---|---|---|
| | λₙₑᵤ^{max} | λₒₓ^{max} (nm) | | Neu. | Ox. |
| Chromophore | (nm) | SOMO-α | SOMO-β | (L*a*b*) | (L*a*b*) |
| 4-FDHPP | 345 | 450 | 650 | 100, | 91, |
| | | | | 0, 0 | −27, 38 |
| F,OMeDHPP | 350 | 445 | 750 | 100, | 94, |
| | | | | 0, 0 | −11, 46 |
| 4-OMeDHPP | 345 | 490 | 740 | 100, | 89, |
| | | | | 0, 2 | −4, 68 |
| 4-SMeDHPP | 370 | 515 | >800 | 100, | 73, |
| | | | | 0, 0 | 20, −27 |
| 4-tol₂ADHPP | 385 | 565 | N/A | 99, | 95, |
| | | | | −1, 4 | −12, 23 |

This shift may be explained by cross-conjugated placement of the electron-donating methoxy substituent that added electron density to the conjugated network. A more pronounced red shift was seen from these three molecules compared to the 4-SMeDHPP with a (λₘₐₓ^{neu}) of about 370 nm and the 4-tol₂ADHPP with (λₘₐₓ^{neu}) of about 385 nm due to the increased electron-donating capabilities of the peripheral substituents.

As it related to potential high-contrast molecules, the experimental and the calculated data agreed with the band gap calculations above that predicted the neutral absorbance would be in the UV-region of the EMS. The experimental oxidized spectra displayed both high- and low-energy transitions and were in suitable agreement with the calculated spectra. Due to the alignment of the calculated and experimental spectra in tandem with the neutral species absorbing in UV-region for a number of DHPPs, further investigation into the formation of the radical cation was performed.

Table 14 shows optical data for all ACE molecules, including the λₘₐₓ and color coordinates for the neutral and oxidized species.

### Example 16

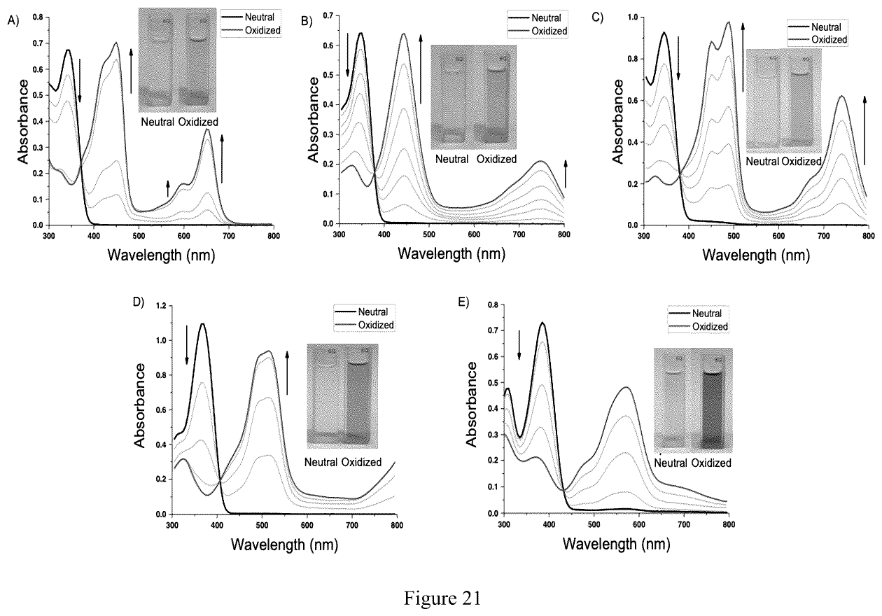

Solution oxidation studies were performed to understand changes in optical properties with increasing levels of oxidation. Solution oxidation studies were performed by titrating DHPP chromophores with a chemical oxidant, and the results are displayed in FIG. 21 and Table 14 above.

Upon oxidation, the five molecules UV-vis absorbances shifted into the visible region of the EMS. F,OMeDHPP had a SOMO-α→LUMO-α similar to that of 4-FDHPP with an (λₘₐₓ^{α}) of about 445 nm and about 450 nm, respectively. On the other hand, 4-OMeDHPP had a red-shifted (λₘₐₓ^{α}) of about 490 nm with characteristics of vibronic fine structure that was attributed potential dimerization.

Changing to a stronger electron-donating substituent, such as 4-SMeDHPP, resulted in a further red shift to a (λₘₐₓ^{α}) of about 515 nm. The SOMO-β→LUMO-β absorbance differed most significantly in the peak shape, with 4-FDHPP having a well-defined peak at about 650 nm with shouldering of about 600 nm, while F,OMeDHPP had a relatively less distinct peak with an (λₘₐₓ^{β}) at about 750 nm. In contrast, 4-OMeDHPP had a well-defined peak at about 740 nm with shouldering of about 675 nm. The shouldering was consistent with dimer being present in the solution as seen in FIG. 21.

The differences in the UV-vis absorbance of the radical cation were consistent with studies studying the effects of electronic influence on positioning of radical cation absorbances. For example, research by Christiansen et al. found that manipulating the electron-donating character at the meta-position resulted in a red shift in the absorbance of the radical cation with increasing electron-donating capabilities.⁶ Overall, the solution oxidation studies of these five molecules demonstrated the ability to manipulate the absorbance of the radical cation of DHPP molecules while maintaining a neutral absorbance in the UV region of the EMS. This shows the optical properties of the electron-withdrawing and -donating substituted DHPPs.

FIG. 21 shows UV-Vis solution oxidation studies of (A) 4-FDHPP, (B) F,OMeDHPP, (C) 4-OMeDHPP, D) 4-SMeDHPP, and E) 4-tol₂ADHPP using 0.06 mg/mL Fe(ClO₄)₃·xH₂O in ethyl acetate as the dopant. This data demonstrates the ability to manipulate the formation of the radical cation by varying the electronic character.

### Example 17

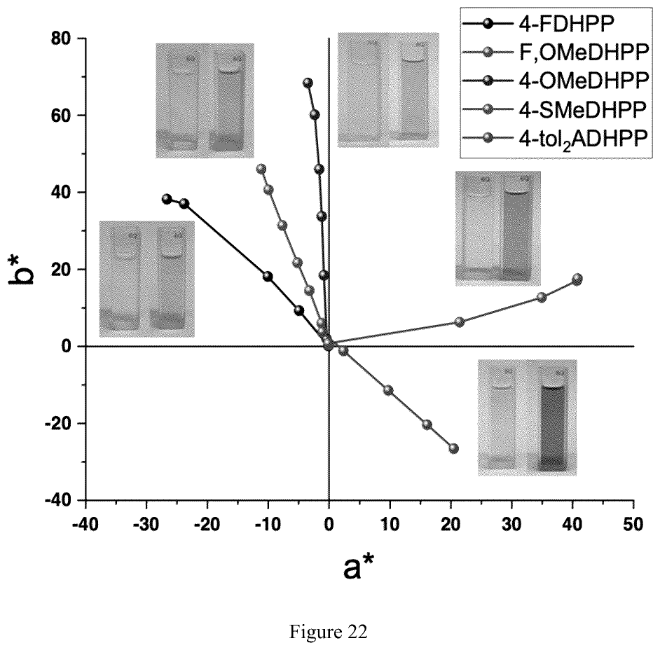

Color coordinates were studied. Observed color changes during titration experiments enabled calculation of color coordinates using the absorbance data collected during solution oxidation studies. Colorimetric analysis based on the Commission Internationale de l'Eclairage 1976 L*a*b* color space was used at a D50 illuminant as a 2° observer to quantify the color of these DHPP chromophores. All five DHPP molecules began in their neutral state at the origin with L*a*b*values of about 100, 0, and 0 (FIG. 22 and Table 14 above). The transmissive solutions are emphasized by the photographs in the insets of FIG. 22.

The radical cation of the DHPP molecules then lied within three different quadrants of the color coordinate diagram with varying relation of proximity to the axis ranging from green to purple. This variation was illustrated by the specific differences in the color values with 4-FDHPP at 91, −27, 38, F,OMeDHPP at 94, −11, 46, 4-OMeDHPP at 89, −4, 68, 4-SMeDHPP at 73, 20, −27, and 4-tol₂ADHPP at 95, −12, 23. The electron-withdrawing 4-FDHPP was green and as the strength of the electron-donating functionality increased, the color coordinates shifted towards the yellow region then the red region and ended within the purple region.

By introducing both an electron-withdrawing and an electron-donating substituent, F,OMeDHPP, the resulting color values shifted to a yellow-green color. Removing the electron-withdrawing functionality shifted the color into the next quadrant with the electron-donating 4-OMeDHPP corresponding to a vibrant yellow. If the electron-donating capabilities were increased further from an oxygen to a sulfur to an aniline, the color red shifted further to lie within the red and then purple region of the color coordinate diagram evidenced by the color of 4-SMeDHPP and 4-tol₂ADHPP, respectively. Due to the transmissive nature of the neutral state for all five DHPPs and their transition to five different colors, they were considered high-contrast. The colorimetry data, in tandem with the CV/DPV and solution oxidation studies, demonstrated that changing the electronic character of the 2,5-position along the DHPP backbone manipulated the radical cation optoelectronic properties.

FIG. 22 shows a color coordinate diagram of 4-FDHPP (black), F,OMeDHPP (red), 4-OMeDHPP (blue), 4-SMeDHPP (green), and 4-tol₂ADHPP (purple) obtained from the solution oxidation studies. All three started at the origin as transmissive, neutral solutions and upon oxidation, tracked away from the origin to produce three distinct colors.

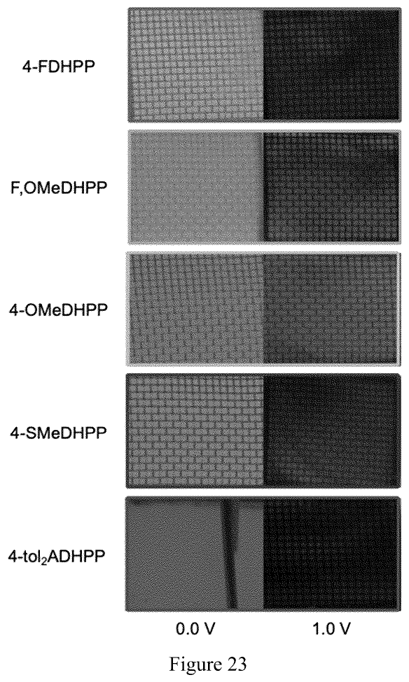

FIG. 23 shows electrochemical switching experiments using an OTTLE of the color-controlled high-contrast DHPP chromophores including the 4-FDHPP, F,OMeDHPP, 4-OMeDHPP, 4-SMeDHPP, and 4-tol₂ADHPP.

### Example 18

Studies of electron-rich pyrrolopyrroles as color-controlled anodically coloring electrochromes were performed. All below examples were performed according to the following materials and methods.

### Materials and Methods

TDDFT calculations using Gaussian 16 and the B3LYP-631G* functional/basis set were performed to elucidate the optical properties of the DHPP molecules that were synthesized. First, the molecules were constructed in Gaussview and a geometry optimization was performed to ensure the correct geometry was used in the subsequent calculations. Next, the excited state calculations were run to understand the positioning of the radical cation absorbance. After completion, the data was collected, normalized to the absorbance maximum, and plotted in Origin to report calculated UV-vis absorbance spectra.

All materials were purchased from commercial sources and used as received unless otherwise stated. Anhydrous dichloromethane (DCM) and toluene were obtained from a Pure Process Technology GC-SPS-7 Glass Contour 800L Solvent Purification System stored under Ar and degassed with argon (Ar) for 15 min. before use. Anhydrous dimethylformamide (DMF) was purchased from Fisher Scientific and was degassed with Ar for 15 min. before use.

¹H NMR and ¹³C NMR spectra were collected on a Bruker Advance III HD 400 MHz NMR spectrometer with nominal concentrations of 5 mg/mL in CDCl₃. Peaks are referenced to the residual CHCl₃ peak (¹H: δ=7.26 ppm; ¹³C: δ=77.23 ppm).

Optical absorbance spectra were acquired using a Varian Cary 60 Scan single-beam UV-vis-near-IR spectrophotometer scanning from 300 to 800 nm. Sample solutions were made to concentrations of 12-24 mM in DCM. Each molecule and polymer were titrated dropwise with a 0.6 mg/mL Fe(ClO₄)₃·xH₂O solution in ethyl acetate until the radical cation peak reached its maximum absorbance intensity. Next to the UV-vis absorbance spectra are photographs taken of neutral and oxidized solutions in quartz cuvettes after the addition of the maximum amount of oxidant. Photographs are presented without manipulation except for cropping.

Colorimetric analysis was performed using a programmed excel sheet based on the Commission Internationale de l'Eclairage 1976 L*a*b* color standards using a D50 illuminant as a 2° observer.

Cyclic voltammetry (CV) and differential pulse voltammetry (DPV) measurements were performed with a CH Instruments electrochemical workstation (CHI660D), using a glassy carbon electrode as the working electrode, an Ag/AgCl reference electrode (calibrated versus the Fc/Fc⁺ redox couple, E₁₂=40 mV), and a Pt flag as the counter electrode. A 50 mV/s scan rate was used for all electrochemical measurements. An electrolyte solution of 0.5 M tetrabutylammonium hexafluorophosphate (TBAPF₆, 98%) in anhydrous DCM was used for all molecular electrochemical measurements.

### Example 19

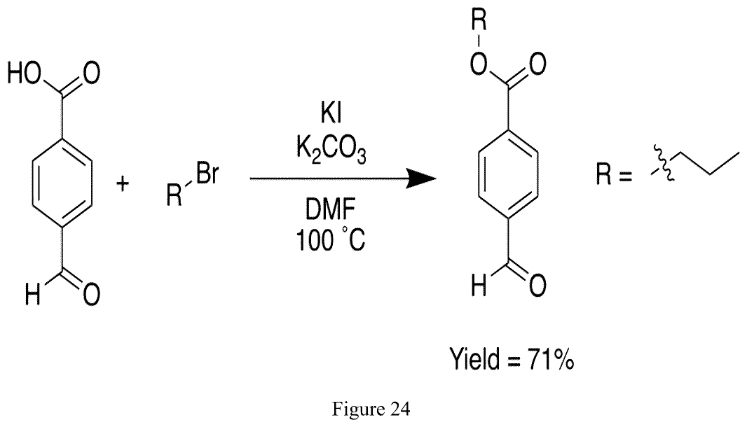

Propyl-4-formylbenzoate was synthesized via Williamson Etherfication. FIG. 24 shows a general reaction scheme for the synthesis of alkylated aldehydes used to synthesize the DHPPs.

4-carboxybenzaldehyde (2.25 g, 15 mmol) was added to a 50 mL 3-neck round bottom flask with 2.5 equiv. of potassium carbonate (K₂CO₃) and a stir bar. The flask was fitted with a condenser, sealed with three rubber septa and was rendered inert via 3× vacuum/refill cycles with Ar. DMF was then added to the reaction flask via cannula for a final concentration of 0.2 M with respect to the aldehyde. This reaction mixture was allowed to stir at r.t. for 30 min. After 30 min, 2.5 equiv. of 1-bromopropane, was added to the reaction flask dropwise over of about 1 min. The reaction flask was then lowered into a thermostatted oil bath and the reaction proceeded at 90° C. of about 17-20 h. The next day, the reaction mixture was allowed to cool to r.t., followed by liquid-liquid extraction with DCM and water (5×) to remove the DMF. The DCM layer containing the product was then dried with sodium sulfate (Na₂SO₄) and filtered via gravity filtration. The solvent was removed via rotary evaporation to obtain the crude product as a brown oil. The product was dried overnight before structural characterization was performed to confirm the desired product was collected with a respectable yield. Gold oil. Yield: 2.05 g (71%). ¹H NMR: (CDCl₃, 400 MHz), δ: 1.04 (t, 3H), 1.82 (m, 2H), 4.32 (t, 2H), 7.95 (d, J=8.4 Hz, 2H), 8.20 (d, J=8.3 Hz, 2H), 10.10 (m, 1H). This NMR spectrum agrees with prior reports.³

### Example 20

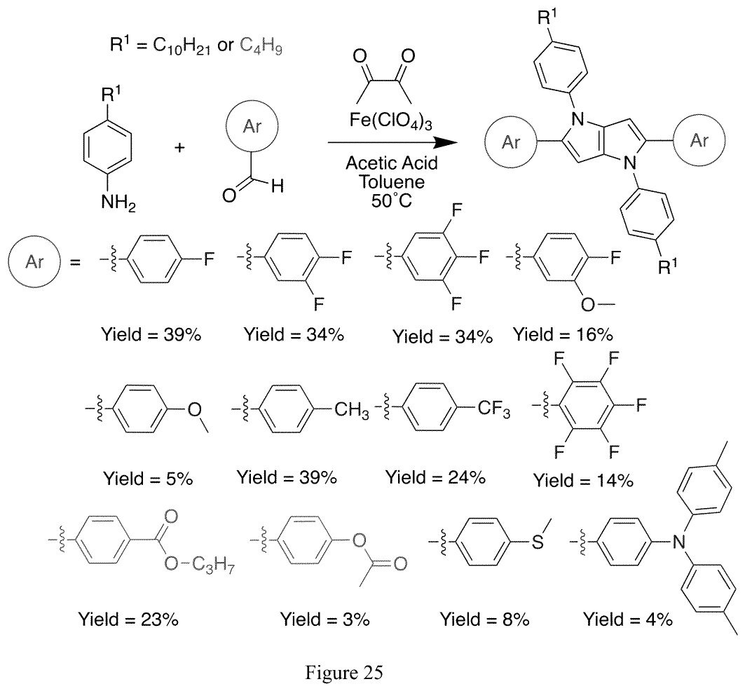

DHPP functionalized molecules and brominated monomers were synthesized via an Fe(III)-catalyzed multicomponent reaction. FIG. 25 shows a reaction scheme for the synthesis of all the DHPP molecules and monomers using the reaction conditions described by Bell et al. Bell, K.-J. J., et al., Chem. Mater., 34: 8729-8739 (2022).

The desired alkyl-aniline (8 mmol) and functionalized benzaldehyde (8 mmol) were added to a solution of toluene (6 mL) and glacial acetic acid (6 mL) inside a 25 mL round bottom flask that was equipped with a magnetic stir bar. This reaction mixture was stirred at 50° C. for 1 h. Once the initial heating time was completed, Fe(ClO₄)₃·xH₂O (0.085 g) was added to the reaction flask, followed by 2,3-butanedione (0.35 mL, 4.00 mmol). After these additions, the reaction was allowed to stir at 50° C. overnight. The next day the reaction was removed from heat and allowed to cool to room temperature. Next, the reaction flask was placed in an ice bath to encourage the formation of a precipitate which was then collected via vacuum filtration followed by washes with cold MeOH and acetone to reveal a white or pale-yellow solid on the filter paper. Finally, the precipitate was transferred to a vial and dried overnight under vacuum. After structural analysis, each molecule was confirmed to be the desired product, as shown by the below analyses.

2,5-bis(4-fluorophenyl)-1,4-bis(4-n-decylphenyl)-1,4-dihydropyrrolo[3,2-b]pyrrole (4-FDHPP): White solid. Yield: 1.13 g (39%). ¹H NMR: (400 MHz, CDCl₃), δ: 0.91 (t, 6H), 1.30-1.36 (m, 30H), 1.62-1.70 (m, 4H), 2.64 (t, 4H), 6.36 (s, 2H), 6.92-6.96 (m, 4H), 7.18-7.22 (m, 13H). ¹³C NMR: (400 MHz, CDCl₃), δ: 14.1, 22.7, 29.4, 29.5, 29.6, 31.4, 31.9, 35.5, 94.3, 115.1 (d, J=21.5 Hz), 125.1, 129.0, 129.8 (d, J=7.8 Hz), 130.0 (d, J=3.3 Hz), 131.3, 134.7, 137.4, 140.6, 161.5 (d, J=245.9 Hz). Anal. calc'd for C₅₀H₆₀F₂N₂ Theory: C, 82.60; H, 8.32; N, 3.85 Actual: C, 82.74; H, 8.37; N, 3.97.

2,5-bis(3,4-difluorophenyl)-1,4-bis(4-n-decylphenyl)-1,4-dihydropyrrolo[3,2-b]pyrrole (3,4-F₂DHPP): Pale-yellow solid. Yield: 1.05 g (34%). ¹H NMR: (400 MHz, CDCl₃), δ: 0.91 (t, 6H), 1.30-1.39 (m, 30H), 1.63-1.70 (m, 4H), 2.64 (t, 4H), 6.36 (s, 2H), 6.91-6.95 (m, 2H), 6.98-7.05 (m, 4H), 7.16-7.22 (m, 9H). ¹³C NMR: (400 MHz, CDCl₃), δ: 14.1, 22.7, 29.3, 29.5, 29.6, 31.3, 31.9, 35.5, 94.7, 116.7, 116.9 (d, J=5.3), 117.1, 124.0, 125.1, 129.2, 130.8 (m, J=7.0 Hz), 131.7, 134.0, 137.0, 141.1, 147.7 (d, J=12.8 Hz), 148.8 (d, J=12.8 Hz), 149.0 (dd, J=248.0 Hz), 150.3 (d, J=12.8 Hz), 151.2 (d, J=12.8 Hz), 150.0 (dd, J=247.0 Hz). Anal. calc'd for C₅₀H₅₈F₄N₂ Theory: C, 78.71; H, 7.66; N, 3.67 Actual: C, 78.71; H, 7.61; N, 3.61.

2,5-bis(3,4,5-trifluorophenyl)-1,4-bis(4-n-decylphenyl)-1,4-dihydropyrrolo[3,2-b]pyrrole (3,4,5-F₃DHPP): Pale-yellow solid. Yield: 1.01 g (34%). ¹H NMR: (400 MHz, CDCl₃), δ: 0.91 (t, 6H), 1.30-1.37 (m, 31H), 1.64-1.71 (m, 4H), 2.68 (t, 4H), 6.36 (s, 2H), 6.77-6.83 (m, 4H), 7.17 (d, J=8.3 Hz, 4H), 7.24 (d, J=8.3 Hz, 4H). ¹³C NMR: (400 MHz, CDCl₃), δ: 14.1, 22.7, 29.3, 29.4, 29.5, 29.6, 31.3, 31.9, 35.5, 95.1, 111.7 (dd, J=22.0 Hz), 125.2, 129.5, 132.2, 133.4, 136.6, 141.6, 150.9 (dm, J=235.0 Hz). Anal. Calc'd for C₅₀H₅₆F₂N₂ Theory: C, 75.16; H, 7.06; N, 3.51 Actual: C, 74.89; H, 6.92; N, 3.57.

2,5-bis(4-fluoro-3-methoxyphenyl)-1,4-bis(4-n-decylphenyl)-1,4-dihydropyrrolo[3,2-b]pyrrole (F,OMeDHPP): Beige solid. Yield: 500 mg (16%). ¹H NMR: (400 MHz, CDCl₃), δ: 0.91 (t, 7H), 1.30-1.39 (m, 32H), 1.61-1.69 (m, 5H), 2.65 (t, 4H), 3.63 (s, 6H), 6.38 (s, 2H), 6.73-6.75 (dd, 2H), 6.81-6.84 (m, 2H), 6.94-6.99 (m, 2H), 7.21 (s, 9H). ¹³C NMR: (400 MHz, CDCl₃), δ: 14.1, 22.7, 29.3, 29.5, 29.6, 31.5, 31.9, 35.5, 55.8, 94.1, 113.5, 115.8 (d, J=18.5 Hz), 120.4 (d, J=6.5 Hz), 125.3, 129.1, 130.2 (d, J=3.8 Hz), 131.4, 135.0, 137.5, 140.8, 147.0 (d, J=10.9 Hz), 151.1 (d, J=245.7 Hz). Anal. Calc'd for C₅₂H₆₄F₂N₂02 Theory: C, 79.35; H, 8.20; N, 3.56 Actual: C, 79.40; H, 8.33; N, 3.69.

2,5-bis(4-methoxyphenyl)-1,4-bis(4-n-decylphenyl)-1,4-dihydropyrrolo[3,2-b]pyrrole (4-OMeDHPP): Yellow solid. Yield: 313 mg (5%). ¹H NMR: (400 MHz, CDCl₃), δ: 0.91 (t, 7H), 1.30-1.39 (m, 35H), 1.62-1.69 (m, 5H), 2.64 (t, 4H), 3.77-3.82 (m, 7H), 6.33 (s, 2H), 6.78-6.81 (m, 4H), 7.16-7.22 (m, 13H). ¹³C NMR: (400 MHz, CDCl₃), δ: 14.1, 22.7, 29.4, 29.5, 29.7, 31.4, 31.9, 35.5, 55.2, 93.8, 113.6, 125.0, 126.7, 128.9, 129.5, 131.0, 135.2, 137.8, 140.2.

2,5-bis(4-meththiophenyl)-1,4-bis(4-n-decylphenyl)-1,4-dihydropyrrolo[3,2-b]pyrrole (4-SMeDHPP): Yellow solid. Yield: 519.1 mg (8%). ¹H NMR (400 MHz, CDCl₃), δ: 0.91 (t, 6H), 1.35 (t, 29H), 1.67 (m, 4H), 2.48 (s, 6H) 2.65 (t, 4H), 6.38 (s, 2H), 7.17 (m, 16H). 13C NMR (400 MHz, CDCl₃), δ: 14.1, 15.8, 22.7, 29.4, 29.5, 29.7, 31.4, 31.9, 35.5, 77.2, 94.3, 125.1, 126.3, 128.4, 129.0, 130.7, 131.7, 135.3, 135.8, 137.6, 140.5. Anal. calc'd for C₅₂H₆₆N₂S₂ Theory: C, 79.74; H, 8.49; N, 3.58; S, 8.19. Actual: C, 79.26; H, 8.38; N, 3.62; S, 8.05.

2,5-bis(diyl)bis(N,N-di-p-tolylaniline)-1,4-bis(4-n-decylphenyl)-1,4-dihydropyrrolo[3,2-b]pyrrole (4-tol₂ADHPP): Yellow solid. Yield: 345.2 mg (4%). ¹H NMR (400 MHz, CDCl₃), δ: 0.90 (t, 8H), 1.30 (m, 37H), 1.64 (m, 5H), 2.32 (s, 15H), 2.64 (t, 5H), 6.34 (s, 2H), 6.87 (d, 6H), 7.05 (dd, 24H), 7.16 (d, 11H). 13C NMR (400 MHz, CDCl₃), δ: 14.1, 20.8, 22.7, 29.4, 29.4, 29.5, 29.6, 29.7, 31.4, 31.9, 35.5, 77.2, 93.7, 122.0, 124.6, 125.1, 127.2, 128.7, 128.8, 129.8, 131.3, 132.4, 135.5, 137.9, 140.2, 145.3, 146.2. Anal. calc'd for C₇₈H₈₈N₄. Theory: C, 88.62; H, 8.20; N, 5.18. Actual: C, 86.68; H, 8.19; N, 5.26.

2,5-bis(tolyl)-1,4-bis(4-n-decylphenyl)-1,4-dihydropyrrolo[3,2-b]pyrrole (4-MeDHPP): Yellow solid. Yield: 683 mg (24%). ¹H NMR: (400 MHz, CDCl₃), δ: 0.92 (t, 7H), 1.31-1.40 (m, 32H), 1.63-1.71 (m, 5H), 2.34 (s, 6H), 2.65 (t, 4H), 6.39 (s, 2H), 7.05 (d, J=8.0 Hz, 4H), 7.14-7.24 (m, 14H). ¹³C NMR: (400 MHz, CDCl₃), δ: 14.1, 21.2, 22.7, 29.4, 29.4, 29.5, 29.7, 31.4, 31.9, 35.5, 77.2, 94.2, 125.1, 128.1, 128.8, 128.9, 131.1, 131.4, 135.6, 135.7, 137.8, 140.2.

2,5-bis(4-trifluoromethylphenyl)-1,4-bis(4-n-decylphenyl)-1,4-dihydropyrrolo[3,2-b]pyrrole (4-CF₃DHPP): Yellow solid. Yield: 1.28 g (39%). ¹H NMR: (400 MHz, CDCl₃), δ: 0.91 (t, 6H), 1.30-1.39 (m, 29H), 1.64-1.71 (m, 4H), 2.67 (t, 4H), 6.48 (s, 2H), 7.19-7.24 (m, 8H), 7.33 (d, J=8.2 Hz, 4H), 7.47 (d, J=8.3 Hz, 4H). ¹³C NMR: (400 MHz, CDCl₃), δ: 14.1, 22.7, 29.4, 29.6, 31.3, 31.9, 35.5, 77.2, 95.4, 125.0-125.2 (m, J=14.1 Hz) 125.1 (d, J=4.0 Hz), 127.8 (d, J=10.1 Hz), 129.3, 132.7, 135.0, 137.0, 137.2, 141.2. Anal. Calc'd for C₅₂H₆₀F₆N₂ Theory: C, 75.52; H, 7.31; N, 3.39 Actual: C, 75.51; H, 7.23; N, 3.40.

2,5-bis(2,3,4,5,6-pentafluorophenyl)-1,4-bis(4-n-decylphenyl)-1,4-dihydropyrrolo[3,2-b]pyrrole (F₅DHPP): Yellow Solid. Yield: 818 mg (14%). ¹H NMR: (400 MHz, CDCl₃), δ: 0.91 (t, 7H), 1.29-1.35 (m, 31H), 1.61-1.69 (m, 4H), 2.64 (t, 4H), 6.48 (s, 2H), 7.15-7.21 (m, 9H). ¹³C NMR: (400 MHz, CDCl₃), δ: 14.1, 22.7, 29.3, 29.5, 29.6, 97.6, 109.2, 119.1, 123.8, 129.3, 131.5, 136.6, 141.5, 145.6. Anal. Calc'd for C₅₀H₅₂F₁₀N₂ Theory: C, 68.95; H, 6.02; N, 3.22 Actual: C, 68.87; H, 6.00; N, 3.22.

2,5-bis(4-acetoxyphenyl)-1,4-bis(4-n-butylphenyl)-1,4-dihydropyrrolo[3,2-b]pyrrole (AcDHPP): White solid. Yield: 18.0 mg (3%). ¹H NMR: (CDCl₃, 400 MHz), δ: 0.95 (t, 6H), 1.38 (m, 6H), 1.63 (m, 4H), 2.28 (s, 6H), 2.62 (t, 4H), 6.36 (s, 2H), 6.95 (m, 4H), 7.20 (m, 12H). ¹³C NMR (CDCl₃, 400 MHz), δ: 14.1, 21.3, 22.5, 33.6, 35.3, 94.8, 121.3, 125.2, 129.1, 129.2, 131.7, 131.8, 135.1, 137.6, 140.7, 149.1, 169.6.

2,5-bis(4-n-propylbenzoate)-1,4-bis(4-n-butylphenyl)-1,4-dihydropyrrolo[3,2-b]pyrrole (EstDHPP): Yellow solid. Yield: 1.28 g (23%). ¹H NMR: (CDCl₃, 400 MHz), δ: 0.99 (m, 12H), 1.40 (m, 4H), 1.64 (m, 4H), 1.78 (m, 4H), 2.65 (t, 4H), 4.25 (t, 4H), 6.48 (s, 2H), 7.19 (m, 8H), 7.27 (d, J=6.7 Hz, 4H), 7.88 (d, J=8.3 Hz, 4H). ¹³C NMR (CDCl₃, 400 MHz), δ: 10.7, 14.2, 22.3, 22.6, 33.7, 35.4, 66.6, 95.7, 125.4, 127.6, 127.8, 129.4, 129.6, 133.2, 135.8, 137.5, 138.1, 141.2. Anal. Calc'd for C₄₆H₅₀N₂O₄ Theory: C, 79.51; H, 7.25; N, 4.03 Actual: C, 79.27; H, 7.37; N, 4.04.

### Example 21

Fluorinated DHPPs were studied. Such studies included computational studies.

TDDFT is a powerful tool in determining the optoelectronic properties of DHPP molecules and polymers such that fundamental structural influences on properties such as absorbance, fluorescence, and redox activity can be studied. Similar calculations were used to elucidate potential structural impact on the optical properties of targeted DHPPs.

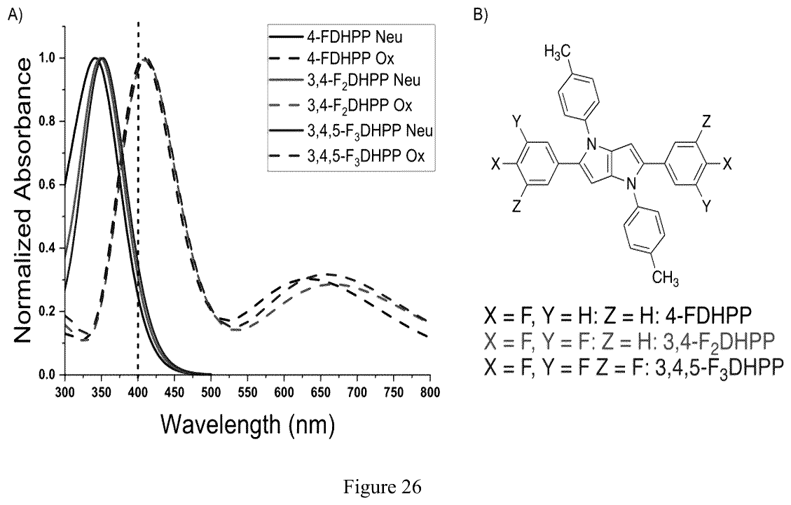

FIG. 26 shows the calculated UV-vis absorbance spectra of three DHPPs with varying degrees of fluorination. The effect of increasing the electron-withdrawing groups on a phenyl ring on the absorbance of the radical cation in DHPP chromophores is studied according to the present disclosure.

The neutral absorbance spectra of this initial set of molecules were all predicted to be positioned in the UV region of the EMS, shown by their similar calculated absorbance maxima (λₘₐₓ) shown in FIG. 26A. This shows absorbance in this region which is the first criterion for anodically coloring electrochromes. However, the electron-withdrawing effect of fluorines at the meta-position did not seem to significantly impact the positioning of the radical cation based on the similar absorbance profiles represented by the dashed lines in FIG. 26A. The high-energy singly occupied molecular orbital transition (SOMO-α) to the lowest unoccupied molecular orbital (LUMO) (SOMO-α→LUMO-α) was about 450 nm for all of the molecules, and the lower-energy SOMO→LUMO transition (SOMO-β→-4 LUMO-β) had a broad peak of about 650 nm. While the predicted spectra did not show a large difference between the set of molecules, the three molecules were synthesized and their structure-property relationships were explored to determine the reliability of TDDFT calculations.

FIG. 26 shows (FIG. 26A) normalized UV-vis absorbance spectra generated from TDDFT calculations of the fluorinated DHPP molecules using B3LYP-631G*. The neutral spectra are the solid lines while the oxidized are dashed. FIG. 26 also shows (FIG. 26B) representative structures of the fluorinated DHPP molecules; the R groups were reduced to —CH₃ groups for simplicity.

### Example 22

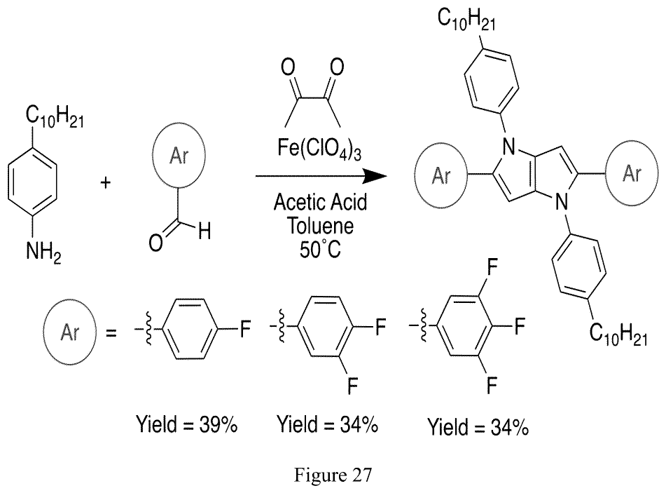

Fluorinated DHPP molecules were synthesized. Theoretical results showed UV-vis absorbance transitions of the neutral state falling within the UV region of the EMS. Three fluorinated DHPP molecules were synthesized using the reaction procedure adopted by Bell et al. and Tasior. Bell, K.-J. J., et al., Chem. Mater., 34: 8729-8739 (2022); Tasior, M., et al., J. Org. Chem., 85: 13529-13543 (2020). The synthesized fluorinated DHPPs were obtained after isolating the compounds via vacuum filtration (FIG. 27). Yields and analytical characterizations were consistent with previously reported DHPP molecules. The molecular structures and purities of the three molecules were confirmed with both NMR and elemental analysis. The ¹H and ¹³C NMR spectra are shown in FIG. 63, 64, 73-76. All protons for each molecule were accounted for within the ¹H NMR spectra, confirming that the desired products were synthesized. The absence of additional peaks supported high compositional purity. The additional peaks in the ¹³C spectra indicated C—F heteronuclear coupling, which further supports the successful incorporation of the fluorinated aldehyde into the DHPP scaffold. The pulse sequence used for the ¹³C measurements did not decouple fluorine and carbon, resulting in C—F couplings constants consistent with reported measurements. Due to this coupling, a further investigation into these results was performed and is reported in Tables 11 above and 15 and 16 below, for 4 for 4-FDHPP, 3,4-F₂DHPP, and 3,4,5-F₃DHPP, respectively.

TABLE 15
J-coupling constants and peak assignments for the
¹³C NMR of 3,4-F₂DHPP. Calculated coupling constants
are consistent with aromatic carbon-fluorine coupling constants
reported by Weigert and coworkers. Weigert, F. J., et al.,
J. Am. Chem. Soc., 93(10): 2361-2369 (1971).

| Chemical Shift | | Coupling | |
|---|---|---|---|
| (ppm) | Peak | Constant J (Hz) | Interaction |
| 150.0 | dd | 247.0 | ¹J (C—F¹) |
| 149.1 | dd | 248.0 | ¹J (C—F²) |
| 151.2, 150.3, | d | 12.8 | ²J (C—C—F BOTH) |
| 148.8, 147.8 | | | |
| 130.8 | m | 7.0 | ³J (C—C—C—F¹) |
| 116.9 | d | 5.3 | ²J (C—C—F²) |

TABLE 16
J-coupling constants and peak assignments for the
¹³C NMR of 3,4,5-F₃DHPP. Calculated coupling constants
are consistent with aromatic carbon-fluorine coupling constants
reported by Weigert and coworkers. Weigert, F. J., et al.,
J. Am. Chem. Soc., 93(10): 2361-2369 (1971).

| Chemical | | Coupling | |
|---|---|---|---|
| Shift (ppm) | Peak | Constant J (Hz) | Interaction |
| 150.9 | dm | 235.0 | ¹J (C—F¹) |
| 111.7 | dm | 22.0 | ²J (C—C—F¹) |
| 111.8 | d | 6.0 | ³J (C—C—C—F¹) |
| 111.6 | d | 6.0 | ³J (V²) |

Overall, the results for the ¹³C NMR were consistent with the expected molecules and, combined with the elemental analysis calculated values matching the theoretical values, further validated the purity of these molecules.

FIG. 27 shows a representative synthetic pathway for the synthesis of fluorinated DHPPs using the Fe(III)-catalyzed multicomponent reaction.

### Example 23

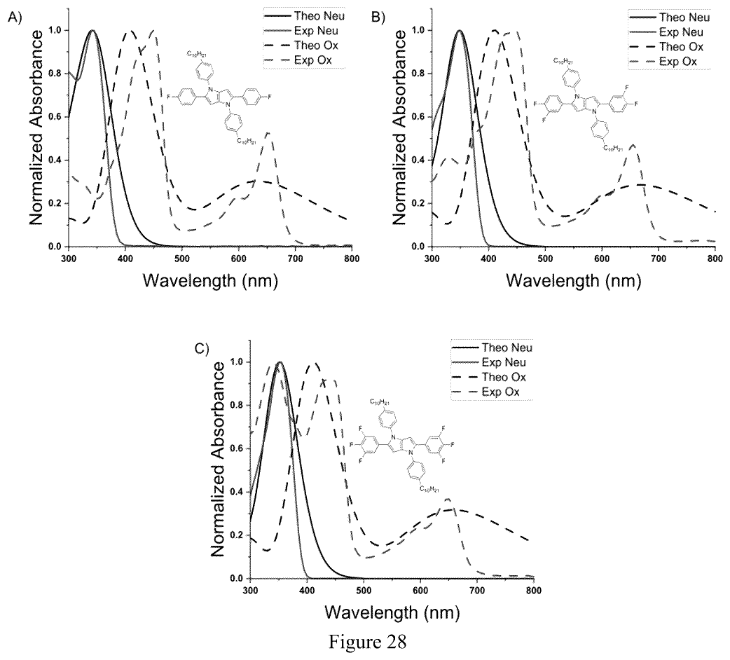

Comparison of calculated and experimental UV-vis absorbance spectra were performed in an effort to verify the level of theory used to predict the UV-vis absorbance of DHPP molecules. The calculated neutral and experimental spectra are represented as the solid lines in FIG. 28, while the dashed lines are the spectra for the oxidized molecules.

Experimentally, the molecules were chemically oxidized using a solution of Fe(ClO₄)₃·xH₂O to generate the radical species. The experimental and calculated neutral spectra for all three fluorinated DHPPs were aligned which indicates the accuracy of the TDDFT level of theory. There was a slight increase in the neutral absorbance maxima wavelength (λₘₐₓ^{neu}) from 4-FDHPP (about 345 nm) to 3,4-F₂DHPP (about 350 nm) to 3,4,5-F₃DHPP (about 355 nm), and this red shift was attributed to the increasing electron-withdrawing nature with increasing fluorination. The results demonstrated that positioning the electron-withdrawing substituents at the meta and ortho positions did not significantly alter the neutral absorbance (FIG. 28) and kept the neutral absorbance in the UV region on the EMS.

On the oxidized spectra, the experimental high energy transition, SOMO-α→LUMO-α, was red shifted compared to the calculated absorbance for each of the molecules resulting in absorbance maxima (λₘₐₓ^{α}) of about 40 nm shifted from calculated to experimental. Such discrepancies in λₘₐₓ of calculated and experimental absorbance could be explained as the predicted UV-vis spectra are Gaussian distributions of the allowed transition.

There was significant overlap of calculated and experimental spectra across the visible spectrum in addition to the accuracy for the calculated neutral spectra. The low energy SOMO-β→LUMO-β transition absorbance maxima (λₘₐₓ^{β}) were similar with the largest difference of these transitions between the molecules coming from the shape of the peaks. The broad theoretical SOMO-β→LUMO-β transition was from TDDFT as an approximation of allowed excited state transitions resulting in a gaussian distribution of the transitions when plotted. The SOMO-β of the experimental UV-vis lied within the broad peak for the calculated and, therefore, was an accurate representation of allowed transitions for the oxidized molecules.

FIG. 28 demonstrates that the theoretical calculations were accurate in predicting the UV-vis absorbance for DHPP molecules in both their neutral and oxidized states which allows design and discovery of novel electrochromes.

FIG. 28 shows calculated vs experimental UV-vis absorbance spectra of (A) 4-FDHPP, (B) 3,4-F₂DHIPP and (C) 3,4,5-F₃DHIPP determining that the level of theory used for TDDFT is reliable and accurate for the neutral and oxidized UV-vis absorbance spectra of DHPP chromophores.

### Example 24

Redox properties were studied. The electrochemical window was expanded for an experimental window from −0.4 V to 2 V vs. Ag/AgCl. The expanded electrochemical window led to the formation of two new oxidation peaks and a shift in the onset of oxidation from of about 0.5 V to of about 0.55 V in addition to the formation of two new reduction peaks at about 0.4 V and about −0.1 V. The results were consistent with reports and supported the formation of new species or the degradation of molecules of the present disclosure. The CV data collected using the expanded experimental window for the rest of the molecules are shown in FIG. 29, and each molecule displays similar trends. These results support needing selection of experimental parameters that will be used for studies of DHPP chromophores.

FIG. 29 shows CV traces of 4-FDHPP demonstrating redox behavior in multiple electrochemical windows. The CV was measured from −0.4 V to 1.2 V (black trace) and −0.4 V to 2 V vs. Ag/AgCl (red trace) using a 0.5 M TBAPF₆/DCM supporting electrolyte.

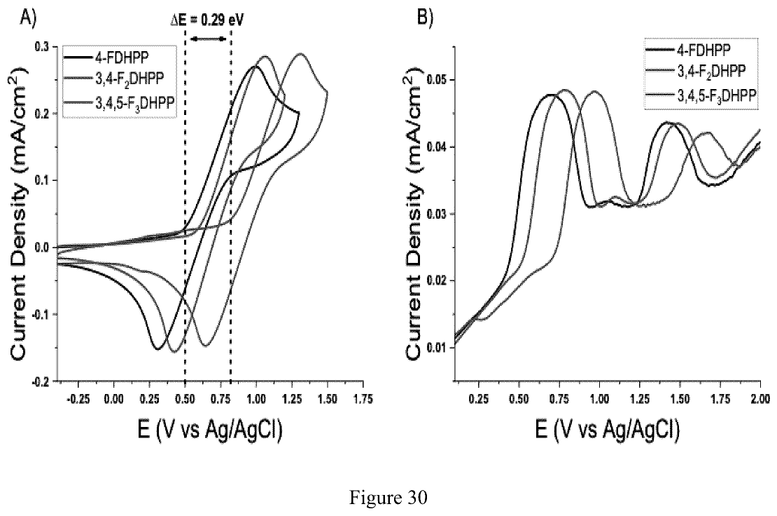

FIG. 30A displays the CV data for all three fluorinated DHPPs and shows the effect of increasing peripheral electronegativity of a DHPP on electrochemical properties. As illustrated in FIG. 30A and tabulated in Table 7 above, an increase in the onset of oxidation (Eₒₙₛₑₜ^{ox}) is seen from 0.42 V, 0.51 V, and 0.70 V (vs. Ag/AgCl) for 4-FDHPP to 3,4-F₂DHPP to 3,4,5-F₃DHPP, respectively (Table 7 above). The increase in Eₒₙₛₑₜ^{ox} from 4-FDHPP to 3,4,5-F₃DHPP (ΔE≈0.29 eV) was attributed to the increase in the electronegativity from the increased fluorine composition on the benzene rings that increased the difficulty of removing an electron from the molecule (FIG. 30A). DPV is has an increased level of precision when determining the onsets of oxidation and therefore gives a more pronounced Eₒₙₛₑₜ^{ox}, as displayed in FIG. 30B. The onsets of oxidation measured via DPV agreed with CV results showing an increase in Eₒₙₛₑₜ^{ox} with increasing electronegativity. DPV also showed the second oxidation peak that was attributed to the formation of the dication of the DHPP molecules (FIG. 30B). While the optical properties were not significantly influenced by the changes in fluorination, the changes in redox response emphasized an influence of peripheral functionalization that allows alternative design strategies to manipulate both optical and redox properties.

FIG. 30 shows (FIG. 30A) CV traces from (−0.4 V to of about 1.5 V) and (FIG. 30B) DPV traces from (−0.4 V to 2 V) of the fluorinated DHPP electrochromes against an Ag/AgCl reference using a 0.5 M TBAPF₆/DCM supporting electrolyte. The least electronegative 4-FDHPP had an onset of 0.42 V while the most electronegative 3,4,5-F₃DHPP had an onset of oxidation of 0.70 V, demonstrating a difference of about 0.29 eV.

Table 7 above shows the onsets of oxidation, calculated energy levels, and energy gaps for fluorinated DHPP molecules. This demonstrates that varying the electron-withdrawing nature of the DHPP periphery altered the redox properties without sacrificing desired optical gaps.

Energy gaps (E_{gap}) and the ionization energies (IE) were estimated given that the IE or, highest occupied molecular orbital (HOMO), is equivalent to Equation 2.2 (Table 7 above). Once the onset of absorbance was obtained through UV-vis spectroscopy, the lowest unoccupied molecular orbital (LUMO) was found through Equation 2.5. Subsequently, the E_{gap} was determined using Equation 2.6. The energy gaps for these DHPPs were calculated to be 3.1 eV and agreed with the molecules absorbing in the UV region of the electromagnetic spectrum (Table 7 above). Given the energy gaps remaining unchanged with varying functionality, these experiments showed peripheral functionalities are capable of manipulating the redox properties of DHPP electrochromes without sacrificing the optical properties required for ACE molecules.

### Example 25

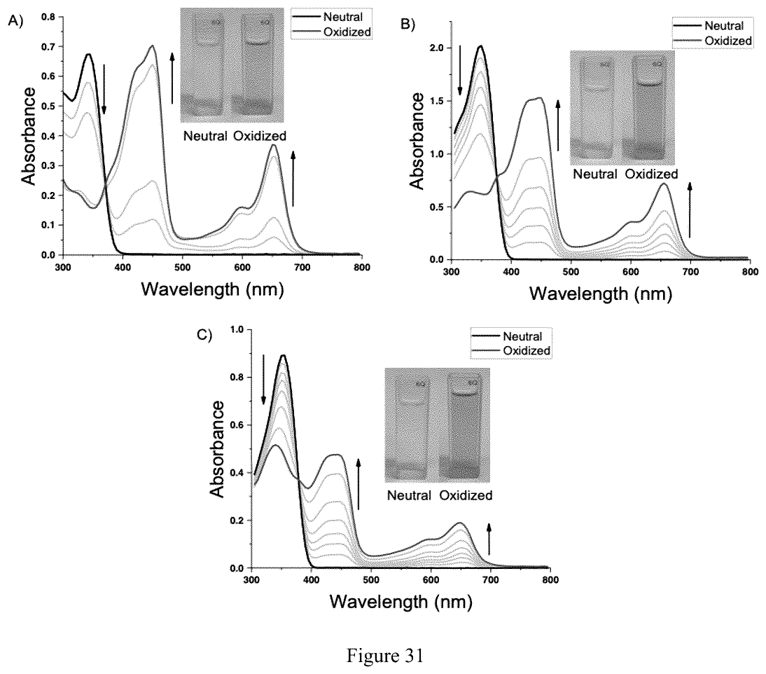

Solution oxidation studies were performed in order to understand changes in optical properties with increasing levels of oxidation. solution oxidation studies were performed by titrating DHPP chromophores with a chemical oxidant. While the absorbance profile of each DHPP was different in intensity and peak shape, all three oxidized DHPPs absorbed between about 400 nm to about 500 nm and about 600 nm to about 700 nm (FIG. 31 and Table 8 above). The (λₘₐₓ^{α}) between about 400 nm up to about 500 nm, or the high energy transition, for 4-FDHPP displayed traits of vibronic fine-structure while 3,4-F₂DHPP and 3,4,5-F₃DHPP both had broad absorbance features. The observed vibronic-fine structure was attributed to dimerization occurring between the oxidized molecules in solution. A difference in the SOMO-β→LUMO-β was in the intensity of the peaks which also was indicative of varying degrees of dimerization. For example, thiophene oligomers with greater charge delocalization have increased charge repulsion resulting in less dimers being formed. Here, as the fluorine substitution was increased, there was a greater push-pull effect which led to increased charge delocalization upon photoexcitation. These results demonstrated that while increasing the electronegativity of DHPP molecules gives variations in the ratios between the SOMO-α→LUMO-α and SOMO-β→LUMO-β of the radical cation, the differences were not significant.

FIG. 31 shows UV-vis solution oxidation spectra of (A) 4-FDHPP, (B) 3,4-F₂DHPP, and (C) 3,4,5-F₃DHPP in DCM using 0.06 mg/mL Fe(ClO₄)₃·xH₂O in ethyl acetate as the dopant. These spectra display changes in the UV-vis absorbance spectra with increasing dopant concentration to elucidate and compare the ability of DHPPs to be chemically doped.

Table 8 above shows optical data for the fluorinated DHPPs, including the absorbance λₘₐₓ and color coordinates for the neutral and oxidized species.

### Example 26

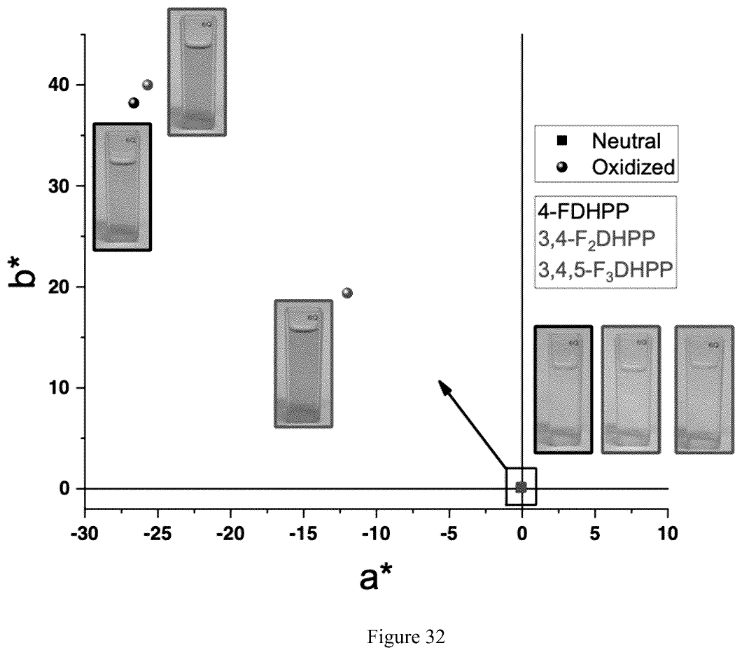

Color coordinates were studied. The observed color changes during titration experiments enabled calculation of color coordinates using the absorbance data collected during solution oxidation studies. Colorimetric analysis based on the Commission Internationale de l'Eclairage 1976 L*a*b* color space was used at a D50 illuminant as a 2° observer to quantify the color of these DHPP chromophores. Upon oxidation, the shifting of the absorbance into the visible region of the EMS led to the transition from the origin to lie between the yellow (+b*) and green (−a*) quadrant of the color coordinate diagram (FIG. 32) and agreed with the observed colors in the photographs of FIG. 31. All three molecules display L* values greater than 90 with only slight variations in their a* and b* values. 4-FDHPP and 3,4-F₂DHPP had a* values of about −27 and b* of about 40 while 3,4,5-F₃DHPP had a*b* of −12, 19, respectively. This difference in quantified color was attributed to the difference in molar absorptivity of the radical cation since all three solutions had the same nominal concentration. The positioning on the coordinate diagram was due to the more prominent absorbance of about 450 nm and the lower energy absorbance of about 650 nm in addition to the minimal absorbance between about 500 nm up to about 570 nm for each chromophore. The colorimetry data confirmed that increasing fluorination at cross-conjugated positions of DHPP functionalities did not significantly alter the optical properties of the radical cation and thus the resulting color. Although adding more fluorines cross-conjugated to the 2,5-positions of DHPP did not alter the overall radical cation absorbance, how to influence the onset of oxidization of anodically coloring electrochromes was elucidated. This allows studies of how to manipulate the structure of DHPPs such that the UV-vis absorbance of the radical cation is controlled while maintaining a relatively low onset of oxidation.

FIG. 32 shows colorimetry data for the three fluorinated DHPPs obtained from the solution oxidation studies. The data shows that increasing the fluorination at the cross conjugated position does not significantly change the color of the radical cation.

### Example 27

The present disclosure relates to expanding the structural diversity of organic electrochromics materials while simultaneously reducing the hazards associated with synthetic protocols. With these considerations in mind, a family of 1,4-dihydropyrrolo[3,2-b]pyrrole (DHPP) chromophores with varying functionalities along the 2,5-axis was envisioned to accomplish these goals. After predicting different absorbance traits as oxidized molecules with time-dependent density functional theory, DHPP chromophores with varying peripheral functionalities were synthesized in a single aerobic synthetic step via an iron-catalyzed multicomponent reaction and characterized as high-contrast chromophores. In solution, the DHPP chromophores absorb in the ultraviolet region of the electromagnetic spectrum, resulting in color-neutral L*a*b* color coordinates of ˜100, 0, 0. Upon chemical oxidation, each molecule transitions to absorb at various points across the visible spectrum based on the extent of electron-donating ability and can display five distinct colors. The chromophores are redox-active and display switching capabilities with an applied electrochemical potential. In conjunction with building fundamental insights into molecular design of DHPP chromophores, the results and synthetic simplicity of DHPPs make them compelling materials for color-controlled high-contrast electrochromes.

Electrochromic materials based on organic molecules and polymers may have many potential applications, including, for example, multifunctional energy storage/conversion/saving devices. Electrochromic materials suffer from drawbacks, such as, for example, residual absorbances that reduce attainable contrasts between oxidation states. Residual absorbances that reduce attainable contrasts between oxidation states is prevalent within a class of electrochromic polymers known as cathodically coloring electrochromes (CCEs) that absorb within the visible region of the electromagnetic spectrum (EMS) as neutral species and transition to absorbing in the infrared region upon oxidation. This residual absorbance is emphasized in many high-contrast CCE polymers based on 3,4-(alkylene)dioxythiophenes (DOTs) and other structures. To overcome this residual absorbance, anodically coloring electrochromes (ACEs) have been developed where molecules absorb in the UV region as neutral molecules and transition to the visible region upon oxidation. By utilizing this approach, ACE molecules achieve true color neutrality in their neutral state with L*a*b* color coordinates of 100, 0, 0 and display systematic color control upon oxidation with stark optical contrast.

While high-contrast materials are attainable, the structural design space for ACE molecules and polymers is quite sparse, and each set of materials comes with its own drawbacks. For example, ACE materials based on triarylamines or poly-(amine-amides) can display high oxidation potentials, possess limited color control, or have poor device bistability. These materials also require numerous synthetic steps to prepare the corresponding monomers. Alkylenedioxypyrroles (DOPs) are also known to be high-contrast electrochromic materials, but their synthesis is relegated to electropolymerization or a stubborn dehalogenation polycondensation reaction. More recently, a series of phenylene-functionalized dioxythiophenes that accomplish high-contrast electrochromism and color control of the radical cation were reported. However, these molecules typically require multiple synthetic steps and use Stille cross-coupling reactions that produce stoichiometric amounts of toxic waste. The combined drawbacks across each class of materials motivate discovering new scaffolds that reduce the synthetic complexity, eliminate the use of toxic reagents, and accomplish this without sacrificing the color control properties of ACE chromophores.

The present disclosure explores the viability of utilizing 1,4-dihydropyrrolo[3,2-b]pyrroles (DHPPs) as synthetically simple monomers that participate in efficient polymerizations to yield simple yet tailorable conjugated polymers. The present disclosure hypothesized that DHPPs would be a useful scaffold to accomplish this goal. A one-step multicomponent reaction to attain DHPPs with properties such as high fluorescence quantum yields and violet, blue, and green fluorescence has been developed. The present disclosure shows that DHPPs reduce the synthetic complexity commonly associated with conjugated polymers, may be designed with structural handles that impart degradability/recyclability, and display multicolored electrochromism based on the choice of monomeric or molecular coupling partners. Upon closer examination of structure-property relationships that dictate optoelectronic properties, the present disclosure shows that with diminishing push-pull nature of electron-rich DHPP chromophores, the absorbance of neutral molecules shifts toward the UV portion of the EMS. Upon oxidation, the absorbance of the radical cation species shifts to the visible, and the positioning and shape of the absorbance profile are dependent on peripheral functionalities. Converting absorbance spectra to L*a*b* color coordinates revealed that two of the molecules possess coordinates of 100, −1, 3, corresponding to highly color-neutral molecules. Encouraged by these results, and as alluded to in FIG. 41, the present disclosure hypothesized that a family of synthetically simple, high-contrast, and color-controlled ACE molecules based on DHPPs was possible. Accomplishing this goal would ultimately eliminate the need for multiple synthetic steps to attain high-contrast ACE materials while simultaneously expanding color control capabilities of DHPP-based electrochromes.

Time-dependent density functional theory (TD-DFT) is a powerful tool used for understanding structure-property relationships of optoelectronic materials. As such, TD-DFT has been utilized in the development of chromophores and polymers that find utility in organic photovoltaics (OPVs), dye-sensitized solar cells (DSSCs), and electrochromics. However, TD-DFT calculations are usually used as supplemental data to support experimental observations. More recently, there is growing interest in utilizing computation to identify potential synthetic targets with desired properties. For example, TD-DFT may be used to predict the behavior of radical cations based on electronic and steric contributions, isomeric effects on optical properties of electrochromic polymers, and elastic constants of crystalline materials. The close agreement between experiment and calculations enables a streamlined approach for designing next-generation optoelectronic materials with reduced waste production and worker-hours in the lab. With these considerations in mind, expanding theory-driven projects may be impactful for the continued development of ACE molecules and polymers.

The design, synthesis, and characterization of a family of DHPP chromophores that display high-contrast electrochromism with systematic color control with low synthetic complexity are reported herein. A theory-guided approach was exploited to predict changes in absorbance properties of neutral and oxidized DHPPs based on peripheral functionalization. Identification of chromophores that are predicted to absorb in the UV portion of the EMS but have different radical cation absorbance spectra ultimately guided synthetic efforts to attain five DHPP chromophores with varying peripheral functionalities via a one-step Fe-catalyzed multicomponent reaction. Electrochemical characterization via cyclic voltammetry (CV) and differential pulse voltammetry (DPV) revealed that increasing the electron-donating nature of the peripheral functional groups lowers the onset of oxidation without sacrificing wide optical bandgaps (˜3.0 eV) that facilitate absorbance in UV region of the EMS. Upon oxidation, absorbances transition to the visible region, where the positioning and shape of the absorbance profile are influenced by the electron-donating or -withdrawing nature of peripheral functional groups. The molecules may be highly color-neutral as neutral solutions with L*a*b* color coordinates ˜100, 0, 0 and transition to distinctly different and vibrant colors ranging from green to purple in the oxidized state. The present disclosure discloses a strategy to attain synthetically simple, high-contrast electrochromes with systematic color control across three color quadrants. Results from the present disclosure represent an expansion in the applicability of DHPP chromophores and further reinforces their utility as functional conjugated scaffolds while simultaneously expanding the structural diversity of ACE molecules.

When envisioning strategies to understand how peripheral substituents of DHPP molecules dictate the position and shape of the resulting radical cation, incorporating substituents that are electron-withdrawing, electron-donating, or a combination of the two seems logical. The present disclosure relates to influencing the optoelectronic properties of π-extended DHPPs and studies of dioxythiophene-based ACE molecules. The present disclosure may involve establishing agreement between theory and experiment by modeling and characterizing a series of fluorinated DHPPs. A thorough discussion of these efforts is reported within FIGS. 50 and 52-55 and Tables 7 and 8. In short, the close agreement between theory and experiment shown in FIG. 52a validated the level of theory (B3LYP 6-31G*) and supports screening additional chromophores.

Five DHPPs with varying electron-donating capabilities were designed based on commercial availability of starting material and modeled with TD-DFT. As shown in FIG. 42, each of the DHPP molecules was predicted to absorb mostly within the UV region of the EMS and achieve the first requirement for anodically coloring materials. After modeling the singly oxidized state for each molecule, the absorbance profiles resemble the dual-band absorbances observed for dioxythiophene-based ACE chromophores that are indicative of SOMO→LUMO transitions. The SOMO-α→LUMO-α (high-energy absorbance) transitions are predicted to appear between 400 and 500 nm, while the intensities (i.e., oscillator strengths) and positions of the SOMO-β→LUMO-β transitions (low-energy absorbance) are more drastically influenced by the electronic character of peripheral substitution. FIG. 42 emphasizes the predicted dependence of the SOMO-β→LUMO-β absorbance on the choice of functionality that will lead to color control of DHPP electrochromes. Specifically, as the electron-donating ability is increased, the SOMO-β→LUMO-β transition increasingly red-shifts into the NIR. In sum, these calculations support the notion that simple alterations to peripheral substituents will enable systematic color control and motivate continued synthesis and elucidation of structure-property relationships of DHPP electrochromes.

Guided by calculations predicting that UV-vis absorbance transitions of the neutral molecules should be positioned within the UV region of the EMS, the five DHPP molecules from FIG. 42 were synthesized using a Fe-catalyzed multicomponent reaction (FIG. 43). The diagnostic DHPP peak at ˜6.5 ppm in the ¹H NMR spectra that corresponds to the two protons on the fused pyrrolopyrrole ring is present for each molecule and supports successful formation of the DHPP chromophores. This is illustrated in the ¹H NMR spectra reported in FIG. 63, for example. Additionally, the multiplicities in the ¹³C NMR spectra for DHPPs with fluorine substituents were analyzed for C—F coupling. The results are reported in Tables 11, 12, 15, and 16, and analyses of the C—F coupling constants are consistent with previous reports. Elemental analysis also was used to confirm the purity of the newly synthesized DHPPs, and the theoretical values matched the experimental ones with a high level of accuracy. Overall, the robust protocol for synthesizing DHPPs yields a structurally diverse family of chromophores suited for elucidating structure-property relationships that progress the development of ACE molecules.

The predicted and experimental optical properties were compared and all of the molecules absorb in the UV region, and the theoretical and experimental spectra are in adequate agreement. In order to understand changes in optical properties with increasing levels of oxidation, solution oxidation studies were performed by titrating DHPP chromophore solutions with a chemical oxidant and monitoring the change in absorbance. The absorbance corresponding to the π-π* transition (black trace) for each molecule diminishes with increasing oxidant concentration while transitions evolve across the visible region of the EMS. F,OMeDHPP has a SOMO-α→LUMO-α similar to that of 4-FDHPP with an Δₘₐₓ^{α} of 445 and 450 nm, respectively. On the other hand, 4-OMeDHPP has a red-shifted Δₘₐₓ^{α} of 490 nm with characteristics of vibronic fine structure that may be attributed to radical dimerization. Increasing the electron-donating strength for 4-SMeDIPP results in a further red shift to a Δₘₐₓ^{α} of 515 nm, and 4-tol₂ADHPP demonstrates an even more pronounced red shift with a Δₘₐₓ^{α} of 565 nm. The SOMO-β→LUMO-β absorbance differs most significantly in the peak shape, with 4-FDHIPP having a well-defined peak at 650 nm with shouldering around −600 nm, while F,OMeDHIPP has a relatively less distinct peak with a Δₘₐₓ^{β} at 750 nm. Alternatively, 4-OMeDHPP has a well-defined peak at 740 nm with shouldering around −675 nm. Furthermore, the increased electron-donating capabilities of 4-SMeDHPP and 4-tol2ADHPP yield a more pronounced red shift of the SOMO-β→LUMO-β absorbance, resulting in a Δₘₐₓ^{β}>800 nm for both molecules. All of these results are tabulated in Table 14. The differences in the UV-vis absorbances of the radical cations are consistent with previous studies investigating the effects of electronic influence on positioning of radical cation absorbances. For example, manipulating the electron-donating or -withdrawing character of benzene units results in shifts in the absorbance of the radical cation. Additionally, choice of peripheral functionality on biphenyl-functionalized DHPPs changed the absorbance profile of neutral and oxidized DHPP chromophores. The neutral absorbance can be recovered with addition of hydrazine, which demonstrates the reversibility of the redox process and is encouraging for repeated switching experiments when incorporated into a device architecture. The reversibility was further studied with ¹H NMR to confirm that the DHPP chromophore was recovered after the doping/dedoping process. The diagnostic pyrrolopyrrole proton at ˜6.37 ppm for 4-FDHPP may disappear after the addition of the Fe oxidant and formation of the radical cation. Furthermore, the protons associated the benzene rings along the 2,5-axis also may be absent, while protons on the 1,4-benzene rings may display line broadening, which is a common phenomenon when studying doped molecules via NMR. Different trends that may be observed for the benzene rings may be attributed to the varying electronic communication along the conjugation pathways and the 2,5-axis contributing to the redox processes more readily than the 1,4-axis. Following the addition of hydrazine, the radical cation may be reduced back to the parent DHPP structure, which may be evidenced by the reappearance of the protons distributed across the 2,5-axis of the molecule, and this may confirm the absence of undesired side reactions during the doping protocols. Overall, the solution oxidation studies of these five molecules demonstrate the ability to manipulate the absorbance of the radical cations of DHPP molecules while maintaining a neutral absorbance in the UV region of the EMS, and make them suited for high-contrast electrochromes.

The changes in absorbance led to distinct color changes of the solutions during oxidation experiments. The observed color changes enabled calculation of color coordinates using the absorbance data collected during solution oxidation studies. Colorimetric analysis based on the Commission Internationale de l'Eclairage 1976 L*a*b* color space was used at a D50 illuminant as a 2° observer to quantify the color of these DHPP chromophores. All five DHPP molecules begin in their neutral state at the origin with L*a*b* values of ˜100, 0, and 0 (Table 14). Color neutrality is defined by a* and b* values falling within the range ±10, while L* values of ˜100 correspond to transmissive samples. As the Fe oxidant is added, the color tracks away from the graph's origin toward the color quadrant that corresponds to the absorbance profile of the radical cation. Excitingly, as shown in Table 14, the color data for this family of DHPP molecules appear across three different quadrants of the color coordinate diagram. This expansion of color control allows for high-contrast DHPP electrochromes that are not restricted to a single color quadrant. The electron-withdrawing 4-FDHPP is green, and as the positioning and strength of the electrondonating functionality changes, the color coordinates shift toward the yellow region, to the red region, and finally within the purple region. These results are in excellent agreement with UV-vis absorbance data that show a red shift in the low-energy transition of the radical cation into the IR. Overall, the colorimetry data confirm the hypothesis that making small changes to the periphery of DHPP chromophores enables systematic color control of high-contrast DHPP-based ACE molecules.

While the optical properties were found to be appropriate for a new class of ACE molecules, the present disclosure sought to elucidate redox properties for electrochemical switching as well. DHPPs are known to be abundantly electron-rich conjugated systems, and electrochemical measurements are warranted due to the reported redox activity of numerous DHPPs. Redox properties for the present disclosure were measured via CV and DPV to understand structural influences on the properties such as the onset of oxidation and reversibility of the DHPP molecules. The ability to manipulate the onset of oxidation of a DHPP through variation of electron-withdrawing or/and -donating groups is shown in Table 13. Specifically, as electron-donating ability is increased, the onset of oxidation is decreased. For electrochemical switching, a reduction accompanies the oxidation for all five DHPPs. The calculated band gaps (˜3.0 eV) agree with optical measurements where the molecules absorb in the UV region. If the electrochemical window is expanded to 2 V, dications form that lead to the degradation of the molecules. Overall, these results establish these DHPPs as electroactive molecules and motivate the continued investigation into color changes with an electrochemical potential for eventual use in electrochromic applications.

After elucidating structure-property relationships that govern color control of DHPP ACE molecules, efforts involved demonstrating that these color changes occur under electrochemical switching conditions. Here, the molecules were dissolved in anhydrous DCM and placed into a SEC-C thin-layer quartz glass spectroelectrochemical cell with a platinum gauze working electrode. The solutions were held at 0.0 V and photographed to emphasize color neutrality before applying a 1.0 V potential until full color saturation was achieved. Each molecule starts as a transmissive solution in its neutral state and shifts to a vibrant color upon application of an electrochemical potential. Attempts to study these materials as thin films were futile because the chromophores are soluble in many organic solvents that are used in electrochemical measurements (i.e., acetonitrile, propylene carbonate, etc.). Ultimately, the high contrast between neutral and oxidized states with an electrochemical potential paves the way for these DHPPs to be incorporated into electrochromic devices.

Organic electrochromes offer the ability to systematically control color properties through the careful choice of structural motifs, but overall optical contrast may also be improved during electrochemical switching protocols. In a broader context for the field of conjugated materials, reducing the synthetic complexity of materials with tailorable properties is highly desired. The present disclosure addresses both of these goals by exploiting the simple synthesis of DHPPs to access a family of highly tailorable ACE molecules in a single step. Attaining these molecules subsequently enables understanding fundamental structure-property relationships of DHPP chromophores in their neutral and oxidized states. A theory-guided approach is utilized to guide synthetic efforts that ultimately create the first examples of high-contrast, color-controlled DHPP electrochromes. TD-DFT calculations provide precedent that subtle changes in electronic character along the 2,5-axis of DHPP chromophores influence the positioning of the radical cation absorbance while neutral molecules absorb in the UV portion of the EMS. Subsequently, a structurally diverse family of DHPP chromophores was synthesized via an Fe-catalyzed multicomponent reaction and isolated with vacuum filtration. Analysis of optoelectronic properties of the DHPP chromophores revealed a relationship between electrochemical and optical properties and peripheral functionality and corroborated TD-DFT calculations. Specifically, with increasing electron-donating ability of peripheral functionalities, the absorbance of the radical cation species red-shifts across the visible spectrum while also having a lower onset of oxidation. Results from optical measurements are quantified in the L*a*b* color space where neutral solutions are highly color-neutral with L*a*b*˜100, 0, 0 and oxidized solutions occupy three distinct color quadrants from green to purple. In sum, results from these efforts provide useful fundamental insights into design-structure-property relationships of DHPP chromophores and inspire the investigation into additional design motifs that expand the color palette of DHPP electrochromes. This work also represents a simplification of the preparation of electrochromic materials. Ultimately, efforts described in the present disclosure reveal new design motifs for high-contrast color-controlled DHPP-based electrochromes and pave the way for incorporating DHPPs into redox devices.

### All Other DHPPs

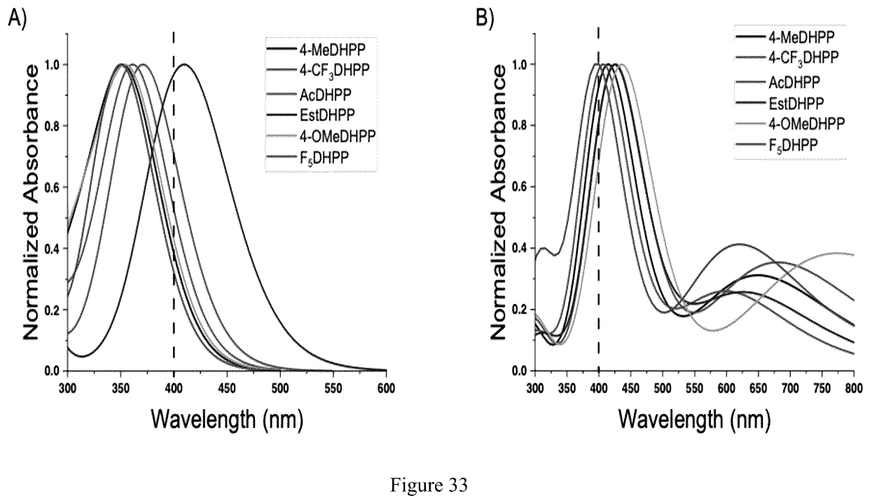

FIG. 33 shows theoretical (FIG. 33A) neutral and (FIG. 33B) oxidized absorbance spectra for seven DHPPs screened for potential color control using B3LYP-631G*. The R groups were reduced to CH₃ groups for simplifying structural input into TDDFT calculations. The neutral spectra are shown as the solid lines while the oxidized are represented as dashed lines.

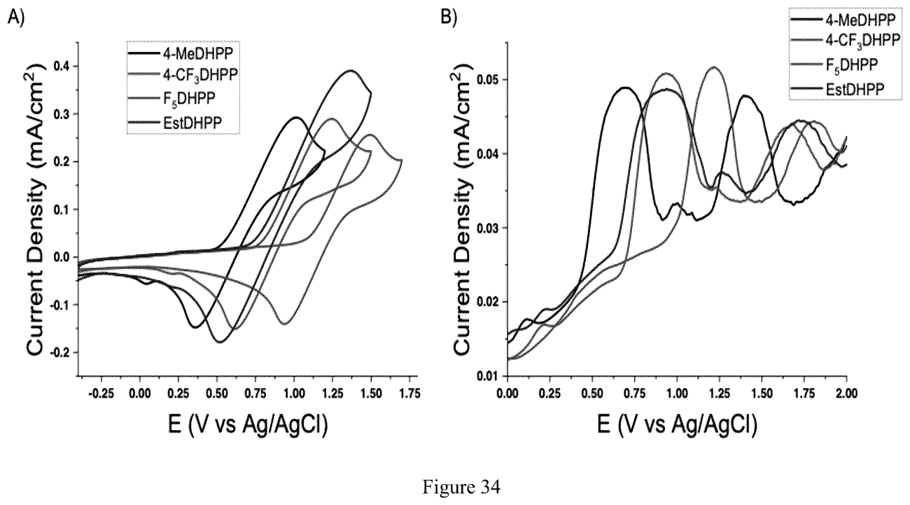

FIG. 34 shows the (FIG. 34A) CV and (FIG. 34B) DPV for the DHPP molecules with various functionalities using an Ag/AgCl reference electrode and a 0.5 M TBAPF₆/DCM supporting electrolyte.

Table 9 above shows electronic properties for DHPPs with varying substituents.

FIG. 35 shows energy level diagrams for DHPP chromophores characterized in this study. While the energy gap is not changing, there is a modulation of the HOMO/LUMO levels resulting from the varying choice of substituents.

Table 10 above shows optical data for all extra ACE molecules, including the λₘₐₓ and color coordinates for the neutral and oxidized species.

All the neutral spectra, except the EstDHPP, absorbed in the UV region between about 330 nm and about 375 nm. The electronically-neutral 4-CH₃DHPP had a (λₘₐₓ^{neu}) of about 350 nm while the electron-donating AcDHPP was slightly red-shifted with a (λₘₐₓ^{neu}) of about 355 nm. The more electron-withdrawing 4-CF₃DHPP had a (λₘₐₓ^{neu}) that as red-shifted to about 375 nm due to the increase in electron-deficiency and increasing the push-pull nature of the molecule. FSDHPP was the most blue-shifted compared to the DHPPs with a (λₘₐₓ^{neu}) of about 330 nm. This blue-shift was hypothesized to be from the increased steric hindrance imparted by the 2 fluorines substituted ortho to the DHPP scaffold. In agreement with calculations, the EstDHPP absorbed in the visible portion of the EMS at about 400 nm due to the lowering of the HOMO/LUMO gap. FSDHPP was not susceptible to chemical oxidization using Fe(ClO₄)₃ likely due to the high electronegativity of the five fluorines (FIG. 36G).

FIG. 36 shows a comparison of calculated and experimental UV-vis absorbance spectra for the seven DHPPs (FIG. 36A) F,OMeDHPP, (FIG. 36B) 4-OMeDHPP, (FIG. 36C) 4-CH₃DHPP, (FIG. 36D) 4-CF₃DHPP, (FIG. 36E) FSDHPP, (FIG. 36F) AcDHPP, and (FIG. 36G) EstDHPP that confirms the level of theory is sufficient in predicting the UV-vis spectra of neutral and oxidized molecules.

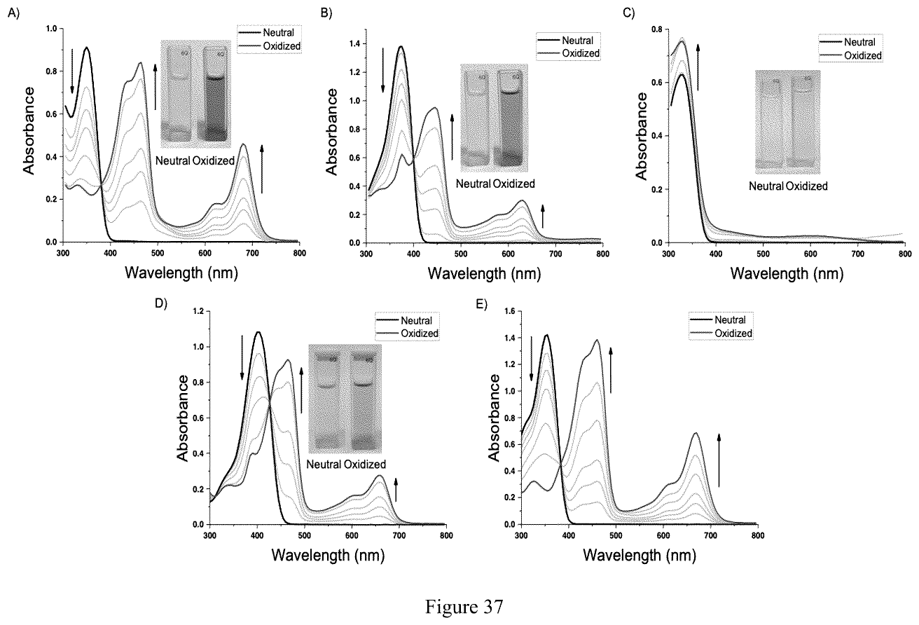

Upon oxidation, the absorbances shifted into the visible with similarly broad SOMO-α→LUMO-α with (λₘₐₓ^{α}) values of about 445 nm up to about 465 nm with some shouldering (FIGS. 61A, 61B, 61D, and 61E) that was consistent with vibronic fine structure being observed for the singly fluorinated DHPP. The SOMO-β→LUMO-β absorbance profiles were all similar with one sharp peak at longer wavelengths and observable shouldering but there were noticeable differences in the location of the SOMO-β→LUMO-β Regarding the set of molecules in FIG. 37, 4-MeDHPP had the most red-shifted (λₘₐₓ^{β}) at about 680 nm while 4-CF₃DHPP had the most blue-shifted (λₘₐₓ^{β}) at about 629 nm and was in agreement with redox characteristics measured during electrochemical experiments. These results indicated increasing the electron-donating capabilities of peripheral functionalities “pushed” the SOMO-β→LUMO-β into the IR region and was consistent with reports. The results show structure-property relationships giving a baseline understanding how changing the electronic character of DHPP molecules, either electronically-withdrawing, -donating, or -neutral, manipulates the optical properties of the radical cation.

FIG. 37 shows solution oxidation studies of (A) 4-MeDHPP, (B) 4-CF₃DHPP, (C) F₅DHPP, (D) EstDHPP, and (E) AcDHPP in DCM using 0.06 mg/mL Fe(ClO₄)₃·xH₂O in ethyl acetate as the dopant.

### NMR

Table 11 above shows J-coupling constants and peak assignments for the ¹³C NMR of 4-FDHPP. Calculated coupling constants were consistent with aromatic carbon-fluorine coupling constants reported by Weigert. Weigert, F. J., et al., J. Am. Chem. Soc., 93(10): 2361-2369 (1971).

Table 12 above shows J-coupling constants and peak assignments for the ¹³C NMR of F,OMeDHPP. Calculated coupling constants were consistent with aromatic carbon-fluorine coupling constants reported by Weigert. Weigert, F. J., et al., J. Am. Chem. Soc., 93(10): 2361-2369 (1971).

Table 15 above shows J-coupling constants and peak assignments for the ¹³C NMR of 3,4-F₂DHPP. Calculated coupling constants were consistent with aromatic carbon-fluorine coupling constants reported by Weigert.

Table 16 above shows J-coupling constants and peak assignments for the ¹³C NMR of 3,4,5-F₃DHPP. Calculated coupling constants were consistent with aromatic carbon-fluorine coupling constants reported by Weigert.

TABLE 17
J-coupling constants and peak assignments for the
¹³C NMR of 4-CF₃DHPP. Calculated coupling constants
are consistent with aromatic carbon-fluorine coupling constants
reported by Weigert and coworkers. Weigert, F. J., et al.,
J. Am. Chem. Soc., 93(10): 2361-2369 (1971).

| Chemical | Coupling | |
|---|---|---|
| Shift (ppm) | Constant J (Hz) | Interaction |
| N/A | N/A | ¹J (C—F) |
| 137.1 | 16.0 | ²J (C—C—F) |
| 125.1 | 14.0 | ²J (C—C—F) |
| 127.8 | 10.0 | ³J (C—C—C—F) |
| 125.1 | 4.0 | ³J (C—C—C—F) |

### Electrochemistry

FIG. 38 shows CV from −0.4 V to 2 V of DHPP molecules included within this disclosure.

FIG. 39 shows DPV traces of the five color-controlled DHPPs according to this disclosure with their onsets of oxidation including A) 4-FDHPP, B) F,OMeDHPP, C) 4-OMeDHPP, D) 4-SMeDHPP, and E) 4-tol₂ADHPP using an Ag/AgCl reference electrode and a 0.5 M TBAPF₆/DCM supporting electrolyte.

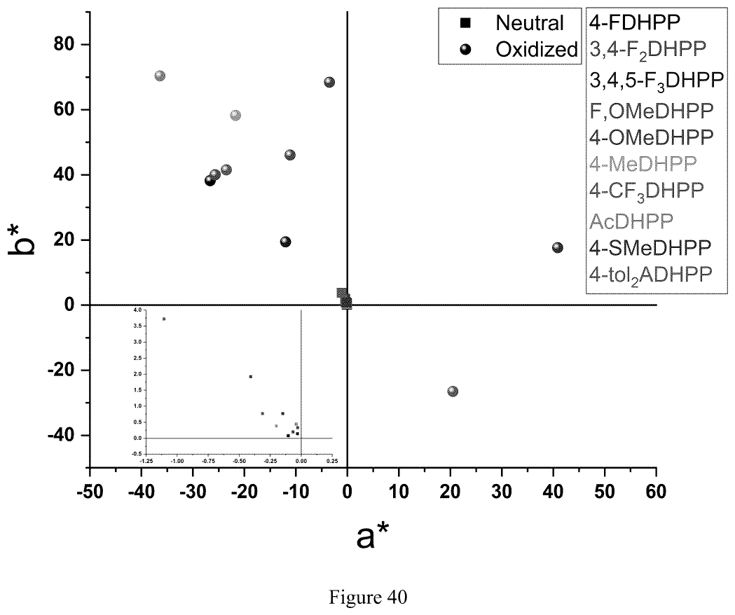

FIG. 40 shows colorimetry data for all ACE DHPP electrochromes according to this disclosure. Each neutral molecule included here displayed a colorless neutral solution and upon oxidation transitioned into the green-yellow region of the color quadrants.

What is claimed is:

1. A composition comprising:
a molecular scaffold capable of being coupled to one or more end groups, wherein the molecular scaffold comprises DHPP or a DHPP derivative; and
the one or more end groups coupled to the molecular scaffold, the one or more end groups selected to control one or more color properties of the composition to result in a high-contrast electrochrome.

2. The composition of claim 1, wherein the one or more end groups is selected from a group consisting of a monocyclic or bicyclic aryl or heteroaryl group.

3. The composition of claim 1, wherein the one or more end groups is selected from a group consisting of a monocyclic or bicyclic aryl or heteroaryl group, and wherein one or more of the monocyclic or bicyclic aryl or heteroaryl group is substituted.

4. The composition of claim 3, wherein the monocyclic or bicyclic aryl or heteroaryl group is substituted with one or more groups selected from an aliphatic, alkenyl, alkynyl, ether, halogenated alkane, cyano, or halogen group.

5. The composition of claim 3, wherein the high-contrast electrochrome is further an anodically coloring electrochrome.

6. The composition of claim 1, wherein the color of the high-contrast electrochrome is capable of being controlled by substituting one or more end groups coupled to the molecular scaffold.

7. The composition of claim 1, wherein the color of the high-contrast electrochrome is capable of being controlled by subjecting the composition to redox reactions.

8. A composition comprising an electrochrome, wherein a color of the electrochrome is capable of being controlled, wherein the electrochrome comprises a molecular scaffold capable of being coupled to one or more end groups, and wherein the molecular scaffold comprises DHPP or a DHPP derivative.

9. The composition of claim 8, wherein the electrochrome is a color-controlled, high-contrast electrochrome.

10. The composition of claim 9, wherein the electrochrome is further an anodically coloring electrochrome.

11. The composition of claim 8, wherein the color of the electrochrome is capable of being controlled by substituting one or more end groups coupled to the molecular scaffold.

12. The composition of claim 8, wherein the color of the electrochrome is capable of being controlled by subjecting the electrochrome to redox reactions.

13. The composition of claim 12, wherein the color of the electrochrome changes as the electrochrome is oxidized or reduced.

14. The composition of claim 13, wherein the color of the electrochrome changes from transmissive to one of a plurality of colors as the electrochrome is progressively oxidized.

15. The composition of claim 14, wherein progressive oxidation occurs via titration.

16. The composition of claim 8, wherein the molecular scaffold is of the following formula: 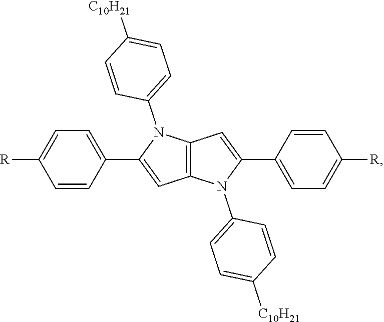
and wherein R can be selectively manipulated to control the color properties of the electrochrome.

17. The composition of claim 16, wherein R is selected from a group consisting of a monocyclic or bicyclic aryl or heteroaryl group.

18. The composition of claim 16, wherein R is selected from a group consisting of a monocyclic or bicyclic aryl or heteroaryl group, and wherein one or more of the monocyclic or bicyclic aryl or heteroaryl group is substituted.

19. The composition of claim 16, wherein the monocyclic or bicyclic aryl or heteroaryl group is substituted with one or more groups selected from an aliphatic, alkenyl, alkynyl, ether, halogenated alkane, cyano, or halogen group.

20. The composition of claim 16, wherein R is selected from the following formulas [chemical structure]
